SEQUENCE LISTING (20 sequences)

SEQ ID NO: 1 - protein, 1049 aa, Bacillus megaterium [sequence omitted]

SEQ ID NO: 2 - protein, 1065 aa, Bacillus cereus
MDKKVSAIPQPKTYGPLGNLPLIDKDKPTLSFIKIAEEYGPIFQIQTLSDTIIVISGHELVAEVCDETRFDKSIEGALAKVRAFAGDGLFTSETQEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSGNQEENDLLSRMLHVQDPETGEKLDDENIRFQIITFLIAGHETTSGLLSFAIYFLLKNPDKLKKAYEEVDRVLTDPTPTYQQVMKLKYIRMILNESLRLWPTAPAFSLYAKEDTVIGGKYPIKKGEDRISVLIPQLHRDKDAWGDNVEEFQPERFEDLDKVPHHAYKPFGNGQRACIGMQFALHEATLVMGMLLQHFEFIDYEDYQLDVKQTLTLKPGDFKIRIVPRNQNISHTTVLAPTEEKLKNHEIKQQVQKTPSIIGADNLSLLVLYGSDTGVAEGIARELADTASLEGVQTEVAALNDRIGSLPKEGAVLIVTSSYNGKPPSNAGQFVQWLEELKPDELKGVQYAVFGCGDHNWASTYQRIPRYIDEQMAQKGATRFSTRGEADASGDFEEQLEQWKESMWSDAMKAFGLELNKNMEKERSTLSLQFVSRLGGSPLARTYEAVYASILENRELQSSSSERSTRHIEISLPEGATYKEGDHLGVLPINSEKNVNRILKRFGLNGKDQVILSASGRSVNHIPLDSPVRLYDLLSYSVEVQEAATRAQIREMVTFTACPPHKKELESLLEDGVYHEQILKKRISMLDLLEKYEACEIRFERFLELLPALKPRYYSISSSPLIAQDRLSITVGVVNAPAWSGEGTYEGVASNYLAQRHNKDEIICFIRTPQSNFQLPENPETPIIMVGPGTGIAPFRGFLQARRVQKQKGMNLGEAHLYFGCRHPEKDYLYRTELENDERDGLISLHTAFSRLEGHPKTYVQHVIKEDRMNLISLLDNGAHLYICGDGSKMAPDVEDTLCQAYQEIHEVSEQEARNWLDRLQDEGRYGKDVWAGI

SEQ ID NO: 3 - protein, 1120 aa, Aspergillus fumigatus
MSESKTVPIPGPRGVPLLGNIYDIEQEVPLRSINLMADQYGPIYRLTTFGWSRVFVSTHELVDEVCDEERFTKVVTAGLNQIRNGVHDGLFTANFPGEENWAIAHRVLVPAFGPLSIRGMFDEMYDIATQLVMKWARHGPTVPIMVTDDFTRLTLDTIALCAMGTRFNSFYHEEMHPFVEAMVGLLQGSGDRARRPALLNNLPTSENSKYWDDIAFLRNLAQELVEARRKNPEDKKDLLNALILGRDPKTGKGLTDESIIDNMITFLIAGHETTSGLLSFLFYYLLKTPNAYKKAQEEVDSVVGRRKITVEDMSRLPYLNAVMRETLRLRSTAPLIAVHAHPEKNKEDPVTLGGGKYVLNKDEPIVIILDKLHRDPQVYGPDAEEFKPERMLDENFEKLPKNAWKPFGNGMRACIGRPFAWQEALLVVAILLQNFNFQMDDPSYNLHIKQTLTIKPKDFHMRATLRHGLDATKLGIALSGSADRAPPESSGAASRVRKQATPPAGQLKPMHIFFGSNTGTCETFARRLADDAVGYGFAADVQSLDSAMQNVPKDEPVVFITASYEGQPPDNAAHFFEWLSALKENELEGVNYAVFGCGHHDWQATFHRIPKAVNQLVAEHGGNRLCDLGLADAANSDMFTDFDSWGESTFWPAITSKFGGGKSDEPKPSSSLQVEVSTGMRASTLGLQLQEGLVIDNQLLSAPDVPAKRMIRFKLPSDMSYRCGDYLAVLPVNPTSVVRRAIRRFDLPWDAMLTIRKPSQAPKGSTSIPLDTPISAFELLSTYVELSQPASKRDLTALADAAITDADAQAELRYLASSPTRFTEEIVKKRMSPLDLLIRYPSIKLPVGDFLAMLPPMRVRQYSISSSPLADPSECSITFSVLNAPALAAASLPPAERAEAEQYMGVASTYLSELKPGERAHIAVRPSHSGFKPPMDLKAPMIMACAGSGLAPFRGFIMDRAEKIRGRRSSVGADGQLPEVEQPAKAILYVGCRTKGKDDIHATELAEWAQLGAVDVRWAYSRPEDGSKGRHVQDLMLEDREELVSLFDQGARIYVCGSTGVGNGVRQACKDIYLERRRQLRQAARERGEEVPAEEDEDAAAEQFLDNLRTKERYATDVFT

SEQ ID NO: 4 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFVRDFAGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 5 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFARDFAGDGLFTSWTHEKNWKKAHNILLPSFSQQAMKGYHAMMVDIAVQLVQKWERLNADEYIEVPEDMTRLTLDTIGLCGFNYRFNSFYRDQPHPFIISMIRALDEVMNKLQRANPDDPAYDENKRQFQEDIKVMNDLVDKIIADRKASGEQSDDLLTQMLNGKDPETGEPLDDGNISYQIITFLIAGHETTSGLLSFALYFLVKNPHVLQKVAEEATRVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 6 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFARDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFIISMVRALDEVMNKLQRANPDDPAYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 7 - protein, 1049 aa, Artificial Sequence
  Engineered sequence
MTIKEMPQPKTFGELKNLPLLNTDKPVQALMKIADELGEIFKFEAPGCVTRYLSSQRLIKEACDESRFDKNLSQALKAVRDFAGDGLITSWTHEINWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 8 - protein, 1049 aa, Artificial Sequence
  Engineered sequence
MTIKEMPQPKTFGELKNLPLLNTDKPVQALMKIADELGEIFKFEAPGCVTRYLSSQRLIKEACDESRFDKNLSQALKAVRDFAGDGLATSWTHEINWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 9 - protein, 1049 aa, Artificial Sequence
  Engineered sequence
MTIKEMPQPKTFGELKNLPLLNTDKPVQALMKIADELGEIFKFEAPGCVTRYLSSQRLIKEACDESRFDKNLSQALKAVRDFLGDGLFTSWTHEINWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 10 - protein, 1049 aa, Artificial Sequence
  Engineered sequence
MTIKEMPQPKTFGELKNLPLLNTDKPVQALMKIADELGEIFKFEAPGCVTRYISSQRLIKEACDESRFDKNLSQPLKFFRDFSGDGLFTSWTHEINWKKAHNILLPSFSQQAMKGYHAMMVDIAVQLVQKWERLNADEHIEVSEDMTRLTLDTIGLCGFNYRFNSFYRDQPHPFIISMVRALDESMNKPQRANPDDPAYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTFPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTGQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGTVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 11 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFARDWSGDGLATSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 12 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFARDSVGDGLATSWTHEKNWKKAHNILLPSFSQQAMKGYHAMMVDIAVQLVQKWERLNADEHIEVSEDMTRLTLDTIGLCGFNYRFNSFYRDQPHPFIISMVRTLDEVMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 13 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFTRDIAGDGLATSWTHEKNWKKAHNILLPSFSQQAMKGYHAMMVDIAVQLVQKWERLNADEHIEVSEDMTRLTLDTIGLCGFNYRFNSFYRDQPHPFIISMVRTLDEVMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 14 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFNRDFAGDGLATSWTHEKNWKKAHNILLPSFSQQAMKGYHAMMVDIAVQLVQKWERLNADEHIEVSEDMTRLTLDTIGLCGFNYRFNSFYRDQPHPFIISMVRTLDEVMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 15 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFNRDFAGDGLATSWTHEKNWKKAHNILLPSFSQQAMKGYHAMMVDIAVQLVQKWERLNADEHIEVSEDMTRLTLDTIGLCGFNYRFNSFYRDQPHPFIISMVRAADESMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 16 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFTRDIAGDGLATSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 17 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFTRDITGDGLATSWTHEKNWKKAHNILLPSFSQQAMKGYHAMMVDIAVQLVQKWERLNADEHIEVSEDMTRLTLDTIGLCGFNYRFNSFYRDQPHPFIISMVRTLDEVMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 18 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFNRDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAADESMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 19 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFTRDITGDGLATSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 20 - protein, 1049 aa, Artificial Sequence
  Engineered sequence
MTIKEMPQPKTFGELKNLPLLNTDKPVQALMKIADELGEIFKFEAPGRVTRYLSSQRLIKEACDESRFDKNLSQALKFTRDITGDGLSTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPVYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

(19) United States
(12) Patent Application Publication
(10) Pub. No.: US 2016/0115508 A1
(43) Pub. Date: Apr. 28, 2016

(54) PARTHENOLIDE DERIVATIVES, METHODS FOR THEIR PREPARATION AND THEIR USE AS ANTICANCER AGENTS

(71) Applicant: UNIVERSITY OF ROCHESTER, Rochester, NY (US)

(72) Inventors: Rudi Fasan, Rochester, NY (US); Craig T. Jordan, Aurora, CO (US); Joshua N. Kolev, Burlington, NJ (US)

(21) Appl. No.: 14/896,221

(22) PCT Filed: Jun. 4, 2014
(86) PCT No.: PCT/US14/40905

§ 371 Date: Dec. 4, 2015

Related U.S. Application Data
(60) Provisional application No. 61/831,756, filed on Jun. 6, 2013.

(51) Int. Cl. C12P 17/18; C12N 9/02; C07D 493/04

(52) U.S. Cl. CPC C12P 17/181; C07D 493/04; C12N 9/0071; C12N 9/0042; C12Y 106/02004; C12Y 114/14001

(57) ABSTRACT

Methods are provided for the generation of parthenolide derivatives functionalized at carbon atoms C9 and C14. Natural cytochrome P450 enzymes, and engineered variants of these enzymes, are used to carry out the hydroxylation of these sites in parthenolide. These P450-catalyzed C—H hydroxylation reactions are coupled to chemical interconversion of the enzymatically introduced hydroxyl group to install a broad range of functionalities at these otherwise unreactive sites of the molecule. The methods can also be used to produce bifunctionalized parthenolide derivatives, which in addition to modifications at the level of carbon atom C9 or C14, are also functionalized at the level of carbon atom C13.

### CROSS-REFERENCE TO RELATED APPLICATIONS

This application claims priority to and the benefit of co-pending U.S. provisional patent application Ser. No. 61/831,756, entitled Parthenolide Derivatives, Methods for Their Preparation and Their Use as Anticancer Agents, filed Jun. 6, 2013, which is incorporated herein by reference in its entirety.

### STATEMENT REGARDING FEDERALLY SPONSORED RESEARCH OR DEVELOPMENT

The disclosed invention was made with government support under contract no. GM098628 from the National Institutes of Health. The government has rights in this invention.

### 1. TECHNICAL FIELD

The present invention relates to derivatives of the sesquiterpene lactone parthenolide, methods and compositions for their preparation, and methods for using the parthenolide derivatives in pharmaceutical compositions as anticancer and anti-inflammatory agents. The invention also relates to engineered cytochrome P450 polypeptide having improved enzyme capability to hydroxylate parthenolide. The invention also relates to methods for producing parthenolide derivatives modified at positions C9 and C14. The invention also relates to methods for producing parthenolide derivatives modified at positions C9 and C14 in conjunction with modifications at position C13, via chemoenzymatic methods. The invention further relates to methods for using parthenolide derivatives for treating cancer and inflammatory diseases.

### 2. BACKGROUND OF THE INVENTION

Parthenolide (1, PTL) is a germacrane sesquiterpene lactone which has been isolated from various genera of the Asteraceae and Magnoliaceae family. Over the past years, this natural product has attracted considerable attention owing to its broad spectrum of biological properties, which include anti-inflammatory (Merfort 2011), antitumor (Ghantous, Gali-Muhtasib et al. 2010; Merfort 2011; Janecka, Wyrebska et al. 2012; Kreuger, Grootjans et al. 2012), antiviral (Hwang, Chang et al. 2006), and antileishmanic (Tiuman, Ueda-Nakamura et al. 2005) activity. The anti-inflammatory properties of parthenolide has been associated to its ability to inhibit the NF-κB transcription factor (Hehner, Heinrich et al. 1998; Garcia-Pineres, Castro et al. 2001; Garcia-Pineres, Lindenmeyer et al. 2004), which plays a prevalent role in regulating inflammatory responses (Baeuerle and Henkel 1994) as well as inhibition of other cellular mechanisms involved in inflammation, such as prostaglandin synthesis and IL-1α expression (Hwang, Fischer et al. 1996) and activation of the NLRP-3 inflammasome (Juliana, Fernandes-Alnemri et al. 2010). Pharmacological inhibition of NF-κB activation, such as that induced by parthenolide, has been recognized as an important strategy for the treatment of a variety of inflammation-related pathologies, including toxic shock, asthma, and rheumatoid arthritis (Barnes and Adcock 1997; Barnes and Larin 1997). Notably, parthenolide has been identified as the major active ingredient of the medicinal herb feverfew (Tanacetum parthenium), which has found use in the traditional medicine for the treatment of pain, migraine, and rheumatoid arthritis (Heptinstall, White et al. 1985; Knight 1995).

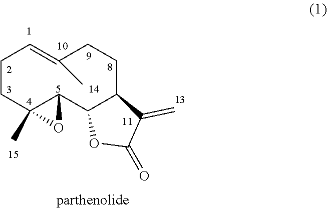 [SMILES: C=C1C(=O)O[C@H]2[C@H]1CC/C(C)=C/CC[C@@]1(C)O[C@@H]21]

Notably, a growing number of studies over the past decade have demonstrated the therapeutic potential of parthenolide also as an anticancer agent. In particular, PTL has emerged as a very promising antileukemic agent owing to its ability to induce robust apoptosis in primary acute myeloid leukemic (AML) cells while exhibiting minimal toxicity toward normal hematopoietic cells (Guzman, Karnischky et al. 2004; Guzman, Rossi et al. 2005; Guzman, Rossi et al. 2006). Most notably, PTL was found to be equally effective amongst all subpopulations found within primary AML specimens, including the so-called leukemia stem cells (LSCs). LSCs typically occur in a quiescent state, which reduces their responsiveness to conventional chemotherapeutic agents that kill actively cycling cells (Holyoake, Jiang et al. 1999; Costello, Mallet et al. 2000; Graham, Jorgensen et al. 2002; Guzman, Swiderski et al. 2002; Guan, Gerhard et al. 2003). Thus, in addition to being involved in the genesis of AML (Lapidot, Sirard et al. 1994; Bonnet and Dick 1997; Cox, Evely et al. 2004), LSCs are believed to play a major role also in clinical relapse of AML patients following traditional chemotherapy (Killmann 1991; van Rhenen, Feller et al. 2005). Thus, the LSC-targeting ability of PTL makes this compound a particularly interesting candidate toward the development of therapeutics for the treatment of AML as well as of other hematologic malignancies.

In addition to AML, PTL has demonstrated activity against numerous other types of cancer. Indeed, recent studies showed that PTL exhibits notable antitumor properties also against breast (Patel, Nozaki et al. 2000; Nakshatri, Rice et al. 2004; Sweeney, Mehrotra et al. 2005; Liu, Lu et al. 2008; Wyrebska, Gach et al. 2012), lung (Zhang, Qiu et al. 2009; Estabrook, Chin-Sinex et al. 2011; Shanmugam, Kusumanchi et al. 2011), prostate (Sun, St Clair et al. 2007; Kawasaki, Hurt et al. 2009; Shanmugam, Kusumanchi et al. 2010; Sun, St Clair et al. 2010), blood (Steele, Jones et al. 2006; Wang, Adachi et al. 2006; Suvannasankha, Crean et al. 2008; Li, Zhang et al. 2012), colon (Zhang, Ong et al. 2004), bladder (Cheng and Xie 2011), liver (Wen, You et al. 2002; Park, Liu et al. 2005; Ralstin, Gage et al. 2006; Kim, Kim et al. 2012), skin (Won, Ong et al. 2004; Won, Ong et al. 2005; Lesiak, Koprowska et al. 2010), brain (Anderson and Bejcek 2008; Zanotto-Filho, Braganhol et al. 2011), pancreas (Kim, Liu et al. 2005; Yip-Schneider, Nakshatri et al. 2005; Yip-Schneider, Wu et al. 2008; Wang, Adachi et al. 2009; Holcomb, Yip-Schneider et al. 2012), kidney (Oka, Nishimura et al. 2007), and bone (Idris, Libouban et al. 2009) cancer.

The anticancer activity of PTL has been primarily linked to its inhibitory activity on NF-κB as this transcription factor controls multiple cellular processes in cancer, including inflammation, transformation, proliferation, angiogenesis, invasion, metastasis, chemoresistance, and radioresistance (Kreuger, Grootjans et al. 2012). However, additional and/or alternative mechanisms of action contribute to PTL anticancer activity, which include activation of p53 (Gopal, Chanchorn et al. 2009), induction of oxidative stress (Wen, You et al. 2002; Zhang, Ong et al. 2004; Wang, Adachi et al. 2006; Sun, St Clair et al. 2010; Shanmugam, Kusumanchi et al. 2011), inhibition of JNK (Nakshatri, Rice et al. 2004) activation of proapoptotic Bcl-2 family members (Zhang, Ong et al. 2004), modulation of exofacial thiols (Skalska, Brookes et al. 2009), and alteration of epigenetic mechanisms via inhibition of DNA methylation (Liu, Liu et al. 2009) and histone deacetylase activity (Gopal, Arora et al. 2007).

From a structure-activity standpoint, the α-methylene-γ-lactone moiety was found to be critically important for PLT pharmacological properties, as reduction of the α,β-unsaturated lactone to give 11,13-dehydroparthenolide results in complete loss of activity (Kwok, Koh et al. 2001; Hwang, Chang et al. 2006; Neelakantan, Nasim et al. 2009). The key functional role of this structural moiety is largely related to its ability to react with nucleophilic sulphydryl groups in the cellular components (e.g., enzymes, proteins, glutathione) targeted by PTL (Garcia-Pineres, Castro et al. 2001; Kwok, Koh et al. 2001; Garcia-Pineres, Lindenmeyer et al. 2004; Skalska, Brookes et al. 2009). The 4,5-epoxide ring was also found to be rather important for PTL anti-inflammatory and cytotoxic activity, as suggested by the comparatively lower activity of a related sesquiterpene lactone, costunolide (Sun, Syu et al. 2003; Siedle, Garcia-Pineres et al. 2004). Finally, our own and previous studies also evidenced the functional importance of the 1,10-double bond in PTL, as indicated by the greatly reduced antileukemic activity of 1,10-epoxy-PTL compared to PTL (Neelakantan, Nasim et al. 2009).

Given the interesting biological properties exhibited by PTL, the development of PTL derivatives with enhanced potency and/or improved drug-like properties would be highly desirable. For example, owing to its scarce water-solubility, PTL suffers from poor oral bioavailability, which limits its utility for therapeutic applications. Previous efforts toward the manipulation of the PTL scaffold have taken advantage of the reactivity of the α-methylene-γ-lactone, resulting in the preparation of various C13-modified PTL derivatives (Guzman, Rossi et al. 2006; Hwang, Chang et al. 2006; Nasim and Crooks 2008; Han, Barrios et al. 2009; Neelakantan, Nasim et al. 2009; Woods, Mo et al. 2011). See also Crooks et. al, U.S. Pat. No. 7,312,242; U.S. Pat. No. 7,678,904; U.S. Pat. No. 8,124,652. For example, substituents containing nucleophilic amine or sulphydryl groups have been added to the α-methylene-γ-lactone of PTL, to give 11,13-dehydro-13-amino- or 11,13-dehydro-13-thiol-parthenolide adducts, respectively. However, since the α-methylene-γ-lactone moiety is crucial for PTL biological activity, these derivatives have often exhibited reduced potency compared to PTL (Guzman, Rossi et al. 2006; Hwang, Chang et al. 2006; Nasim and Crooks 2008; Han, Barrios et al. 2009; Neelakantan, Nasim et al. 2009; Woods, Mo et al. 2011). The most promising PTL derivative to arise from these studies is 11,13-dehydro-13-dimethylamino-parthenolide (called DMAPT or LC-1) (Guzman, Rossi et al. 2006; Neelakantan, Nasim et al. 2009). DMAPT retains anticancer activity comparable to PTL, while exhibiting higher water solubility and improved oral bioavailability (Guzman, Rossi et al. 2006). DMAPT has advanced to Phase 1 clinical trials for the treatment of hematologic malignancies. In other previous studies, parthenolide has been subjected to other chemical transformations, all of which have resulted in important structural rearrangement of PTL scaffold (Castaneda-Acosta, Fischer et al. 1993; Neukirch, Kaneider et al. 2003; Nasim and Crooks 2008).

Citation or identification of any reference in Section 2, or in any other section of this application, shall not be considered an admission that such reference is available as prior art to the present invention.

### 3. SUMMARY OF THE INVENTION

An engineered cytochrome P450 polypeptide is provided having an improved enzyme capability, as compared to a P450 enzyme of SEQ ID NO: 1, SEQ ID NO: 2, or SEQ ID NO: 3, to hydroxylate parthenolide, wherein the engineered cytochrome P450 polypeptide comprises an amino acid sequence that is at least 60% identical to SEQ ID NO: 1, 2 or 3.

In one embodiment of the polypeptide, the improved enzyme capability of the polypeptide is an improvement in its catalytic activity, coupling efficiency, regioselectivity and/or stereoselectivity.

In another embodiment of the polypeptide, the catalytic efficiency of the polypeptide is at least 1.1-fold, 2-fold, 5-fold, 10-fold, 100-fold, 200-fold, 500-fold, or greater than 500-fold higher than the catalytic efficiency of its respective naturally occurring parental sequence SEQ ID NO: 1, SEQ ID NO: 2, or SEQ ID NO: 3.

In another embodiment of the polypeptide, the polypeptide comprises an amino acid sequence that is at least 90% identical to SEQ ID NO: 1 and comprises an amino acid substitution at a position selected from the group consisting of position X26, X27, X43, X48, X52, X53, X73, X75, X76, X79, X82, X83, X88, X89, X95, X97, X143, X146, X176, X181, X182, X185, X189, X198, X206, X226, X227, X237, X253, X256, X261, X264, X265, X268; X269, X291, X320, X331, X329, X330, X354, X355, X367, X394, X435, X436, X444, X446, X438, and X439 of SEQ ID NO: 1.

In another embodiment of the polypeptide, the polypeptide comprises an amino acid sequence that is at least 90% identical to SEQ ID NO: 2 and comprises an amino acid substitution at a position selected from the group consisting of position X28, X29, X45, X50, X54, X55, X75, X77, X78, X81, X83, X85, X90, X91, X97, X99, X145, X148, X178, X183, X184, X187, X191, X200, X208, X228, X229, X240, X256, X259, X264, X267, X268, X271, X272, X294, X323, X334, X332, X333, X358, X359, X371, X398, X439, X440, X448, X440, X442, and X443 of SEQ ID NO: 2.

In another embodiment of the polypeptide, the polypeptide comprises an amino acid sequence that is at least 90% identical to SEQ ID NO: 3 and comprises an amino acid substitution at a position selected from the group consisting of position X29, X30, X46, X51, X55, X56, X76, X78, X79, X82, X85, X86, X91, X92, X99, X101, X147, X151, X180, X185, X186, X189, X193, X202, X210, X230, X231, X241, X257, X260, X265, X268, X269, X272, X273, X295, X324, X335, X333, X334, X365, X366, X378, X405, X446, X447, X455, X457, X449, and X450 of SEQ ID NO: 3.

In another embodiment of the polypeptide, the improved capability is an improved capability to hydroxylate position 9, position 14, or both of these positions in parthenolide.

In another embodiment of the polypeptide, the improved capability in hydroxylating position 14 in parthenolide is an increase in total turnover numbers supported by the enzyme for the oxidation of parthenolide, or an increase in the regioselectivity of the enzyme-catalyzed reaction toward 14-hydroxylation, or both.

In another embodiment of the polypeptide, the improved capability in hydroxylating position 9 in parthenolide is:
(a) an increase in total turnover numbers supported by the enzyme for the oxidation of parthenolide,
(b) an increase in the regioselectivity of the enzyme-catalyzed reaction toward 9-hydroxylation,
(c) an increase in the stereoselectivity of the enzyme-catalyzed reaction toward 9-hydroxylation, or
(d) a combination of (a), (b) and/or (c).

In another embodiment of the polypeptide, the polypeptide is selected from the group consisting of SEQ ID NO: 4, 5, 6, 7, 8, 9, 10, 11, 12, 13, 14, 15, 16, 17, 18, 19, and 20.

In another embodiment of the polypeptide, the polypeptide comprises an amino acid sequence that is at least 90% identical to SEQ ID NO: 1 and comprises at least one of the features selected from the group consisting of: X48 is R or C; X53 is L or I; X75 is A, P, V, or T; X79 is V, A, T, N, or F; X82 is F, I, A, S, or W; X83 is A, L, S, V, or T; X88 is F, A, I, S, or V; X95 is K or I; X139 is H or Y; X143 is P or S; X176 is T or I; X181 is A or T; X182 is L or A; X185 is A, V or S; X189 is L or P; X198 is A or V; X206 is F or C; X227 is S or R; X237 is Q or H; X253 is G or E; X256 is S or R; X291 is V or A; X329 is V or A; X354 is V or L; X367 is I or V; X464 is E or G; and X710 is I or T of SEQ ID NO: 1.

In another embodiment of the polypeptide, the polypeptide comprises an amino acid sequence that is at least 90% identical to SEQ ID NO: 2 and comprises at least one of the features selected from the group consisting of: X81 is V or A; X85 is A or P; X90 is F or A; X184 is L or A; and X187 is A or L of SEQ ID NO: 2.

In another embodiment of the polypeptide, the polypeptide comprises an amino acid sequence comprising a cytochrome P450 heme domain that is at least 60% identical to the amino acid sequence from X1 to X500 in SEQ ID NO: 1, 2, 3, 4, 5, 6, 7, 8, 9, 10, 11, 12, 13, 14, 15, 16, 17, 18, 19, and/or 20.

A method is provided for hydroxylating parthenolide, comprising the steps of:
a. contacting parthenolide with a cytochrome P450 polypeptide of claim 1 under reaction conditions suitable for catalyzing hydroxylation of parthenolide;
b. catalyzing the hydroxylation of parthenolide, while preserving the α-methylene-γ-lactone moiety therein, thereby producing an hydroxylated derivative of parthenolide; and
c. isolating the hydroxylated derivative of parthenolide.

In one embodiment of the method, the polypeptide comprises an amino acid sequence that is at least 90% identical to SEQ ID NO: 1 and comprises an amino acid substitution at a position selected from the group consisting of position X26, X27, X43, X48, X52, X53, X73, X75, X76, X79, X82, X83, X88, X89, X95, X97, X143, X146, X176, X181, X182, X185, X189, X198, X206, X226, X227, X237, X253, X256, X261, X264, X265, X268, X269, X291, X320, X331, X329, X330, X354, X355, X367, X394, X435, X436, X444, X446, X438, and X439 of SEQ ID NO: 1.

In another embodiment of the method, the polypeptide comprises an amino acid sequence that is at least 90% identical to SEQ ID NO: 1 and comprises at least one of the features selected from the group consisting of: X48 is R or C; X53 is L or I; X75 is A, P, V, or T; X79 is V, A, T, N, or F; X82 is F, I, A, S, or W; X83 is A, L, S, V, or T; X88 is F, A, I, S, or V; X95 is K or I; X139 is H or Y; X143 is P or S; X176 is T or I; X181 is A or T; X182 is L or A; X185 is A, V or S; X189 is L or P; X198 is A or V; X206 is F or C; X227 is S or R; X237 is Q or H; X253 is G or E; X256 is S or R; X291 is V or A; X329 is V or A; X354 is V or L; X367 is I or V; X464 is E or G; and X710 is I or T of SEQ ID NO: 1.

In another embodiment of the method, the polypeptide is selected from the group consisting of SEQ ID NO: 4, 5, 6, 7, 8, 9, 10, 11, 12, 13, 14, 15, 16, 17, 18, 19, and 20.

In another embodiment of the method, the polypeptide comprises an amino acid sequence that is at least 90% identical to SEQ ID NO: 2 and comprises an amino acid substitution at a position selected from the group consisting of position X28, X29, X45, X50, X54, X55, X75, X77, X78, X81, X83, X85, X90, X91, X97, X99, X145, X148, X178, X183, X184, X187, X191, X200, X208, X228, X229, X240, X256, X259, X264, X267, X268, X271, X272, X294, X323, X334, X332, X333, X358, X359, X371, X398, X439, X440, X448, X440, X442, and X443 of SEQ ID NO: 2.

In another embodiment of the method, the polypeptide comprises an amino acid sequence that is at least 90% identical to SEQ ID NO: 2 and comprises at least one of the features selected from the group consisting of: X81 is V or A; X85 is A or P; X90 is F or A; X184 is L or A; and X187 is A or L of SEQ ID NO: 2.

In another embodiment of the method, the polypeptide comprises an amino acid sequence that is at least 90% identical to SEQ ID NO: 3 and comprises an amino acid substitution at a position selected from the group consisting of position X29, X30, X46, X51, X55, X56, X76, X78, X79, X82, X85, X86, X91, X92, X99, X101, X147, X151, X180, X185, X186, X189, X193, X202, X210, X230, X231, X241, X257, X260, X265, X268, X269, X272, X273, X295, X324, X335, X333, X334, X365, X366, X378, X405, X446, X447, X455, X457, X449, and X450 of SEQ ID NO: 3.

In another embodiment of the method, the hydroxylated products comprise at least one compounds selected from the group consisting of 14-hydroxyparthenolide, 9(S)-hydroxyparthenolide, and 9(R)-hydroxyparthenolide.

In another embodiment of the method, the polypeptide is tethered to a solid support.

In another embodiment of the method, the solid support is selected from the group consisting of a bead, a microsphere, a particle, a surface, a membrane, a matrix, and a hydrogel.

In another embodiment of the method, the polypeptide is contained in a host cell.

In another embodiment of the method, the host cell is selected from the group consisting of a bacterial cell, a yeast cell, and a plant cell.

A compound of formula (I) or formula (II) is provided

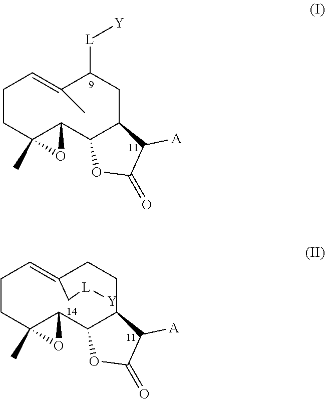

wherein:
A is ═CH₂ or —CH₂R* wherein R* is an amino acid residue bonded to the A methylene via a nitrogen or sulfur atom; or R* is —NR¹R², —NR¹C(O)R², —NR¹CO₂R², or —SR¹, wherein R¹ and R² are independently selected from the group consisting of H and an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or where R* is —NR¹R², R₁ and R₂ optionally together with the nitrogen atom form a an optionally substituted 5-12 membered ring, the ring optionally comprising at least one heteroatom or group selected from the group consisting of —CO—, —SO—, —SO₂—, and —PO—;
L is −O—, —NH—, —NHC(O)—, —OC(O)—, —OC(O)NH—, —S—, —SO—, —SO₂—, —PO—, —OCH₂—, or a chemical bond connecting the carbon atom to Y; and Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or
Y is absent and L represents a halogen atom, an azido group (—N₃), an optionally substituted triazole group, or a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R³ and R⁴ are connected together to form an optionally substituted heterocyclic group; or a pharmaceutically acceptable salt thereof.

In one embodiment of the compound, L is —OC(O)—, Y is selected from the group consisting of phenyl, 4-pyridyl, (4-dimethylamino)phenyl, para-, meta-, and ortho-fluoro-phenyl, para-, meta-, and ortho-trifluoromethyl-phenyl, (2,4-bis-trifluoromethyl)phenyl, (3,5-bis-trifluoromethyl)phenyl, 1- and 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene, and A is ═CH₂.

In another embodiment of the compound, L is —OC(O)—, Y is selected from the group consisting of phenyl, 4-pyridyl, (4-dimethylamino)phenyl, para-, meta-, and ortho-fluoro-phenyl, para-, meta-, and ortho-trifluoromethyl-phenyl, (2,4-bis-trifluoromethyl)phenyl, (3,5-bis-trifluoromethyl)phenyl, 1- and 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene, and A is —CH₂R*, where R* is selected from the group consisting of methylamino (—NH(CH₃)), dimethylamino (—N(CH₃)₂), methylethylamino (—N(CH₃)(CH₂CH₃)), methylpropylamino (—N(CH₃)(CH₂CH₂CH₃)), methylisopropylamino (—N(CH₃)(CH₂(CH₃)₂), —N(CH₃)(CH₂CH₂OH), pyrrolidine, piperidine, 4-methylpiperidine, 1-phenylmethanamine (—NCH₂Ph), and 2-phenylethanamine (—NCH₂CH₂Ph).

In another embodiment of the compound, L is —O—, Y is selected from the group consisting of (phenyl)methyl, (4-pyridyl)methyl, (4-dim ethylaminophenyl)methyl, (para-, meta-, and ortho-fluoro-phenyl)methyl, (para-, meta-, and ortho-trifluoromethyl-phenyl)methyl, (2,4-bis-trifluoromethyl-phenyl)methyl, (3,5-bis-trifluoromethyl-phenyl)methyl, (naphyl)methyl, (3-N-methyl-indolyl)methyl, (5-(4-chlorophenyl)isoxazolyl)methyl, (2-(4-bromophenyl)furanyl)methyl, (2-(2-(trifluoromethyl)phenyl)furanyl)methyl, methyl(thiophene) and —CH(Ar′)COOR′ group, where Ar′ is selected from the group consisting of phenyl, 4-pyridyl, (4-dimethylamino)phenyl, para-, mew-, and ortho-fluoro-phenyl, para-, meta-, and ortho-trifluoromethyl-phenyl, (2,4-bis-trifluoromethyl)phenyl, (3,5-bis-trifluoromethyl)phenyl, 1- and 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene group, and R′ is selected from the group consisting of methyl, ethyl, propyl, isopropyl, tert-butyl, benzyl, 2-morpholinoethyl, 2-morpholinoethyl, 2-(piperidin-1-yl)ethyl, and 2-(pyrrolidin-1-yl)ethyl; and A is ═CH₂.

In another embodiment of the compound, L is —O—, Y is selected from the group consisting of (phenyl)methyl, (4-pyridyl)methyl, (4-dimethylaminophenyl)methyl, (para-, mew-, and ortho-fluoro-phenyl)methyl, (para-, meta-, and ortho-trifluoromethyl-phenyl)methyl, (2,4-bis-trifluoromethyl-phenyl)methyl, (3,5-bis-trifluoromethyl-phenyl)methyl, (naphyl)methyl, (3-N-methyl-indolyl)methyl, (5-(4-chlorophenyl)isoxazolyl)methyl, (2-(4-bromophenyl)furanyl)methyl, (2-(2-(trifluoromethyl)phenyl)furanyl)methyl, methyl(thiophene) and —CH(Ar′)COOR′ group, where Ar′ is selected from the group consisting of phenyl, 4-pyridyl, (4-dimethylamino)phenyl, para-, meta-, and ortho-fluoro-phenyl, para-, meta-, and ortho-trifluoromethyl-phenyl, (2,4-bis-trifluoromethyl)phenyl, (3,5-bis-trifluoromethyl)phenyl, 1- and 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene group, and R′ is selected from the group consisting of methyl, ethyl, propyl, isopropyl, tert-butyl, benzyl, 2-morpholinoethyl, 2-morpholinoethyl, 2-(piperidin-1-yl)ethyl, and 2-(pyrrolidin-1-yl)ethyl; and A is —CH₂R*, where R* is selected from the group consisting of methylamino (—NH(CH₃)), dimethylamino (—N(CH₃)₂), methylethylamino (—N(CH₃)(CH₂CH₃)), methylpropylamino (—N(CH₃)(CH₂CH₂CH₃)), methylisopropylamino (—N(CH₃)(CH₂(CH₃)₂), —N(CH₃)(CH₂CH₂OH), pyrrolidine, piperidine, 4-methylpiperidine, 1-phenylmethanamine (—NCH₂Ph), and 2-phenylethanamine (—NCH₂CH₂Ph).

In another embodiment of the compound, the compound is a pharmaceutically acceptable salt selected from the group consisting of hydrochloride, maleate, fumarate, or mesylate.

A pharmaceutical composition is provided comprising the compound or a pharmaceutically acceptable salt thereof; in combination with a pharmaceutically effective diluent or carrier.

A method of inhibiting cancer cell growth is provided, comprising administering to a mammal afflicted with cancer, an amount of the compound effective to inhibit the growth of the cancer cell(s).

A method of inhibiting cancer cell growth is provided, comprising contacting the cancer cell in vitro or in vivo with an amount of the compound effective to inhibit the growth of the cancer cell.

A method of treating an inflammatory condition is provided, comprising administering to a mammal in need thereof, an amount of the compound effective to reduce, prevent, or control the condition.

In one embodiment of the method, the inflammatory condition is an autoimmune or autoinflammatory disease or disorder, the method comprising administering to a mammal an amount of the compound effective to reduce, prevent, or control the autoimmune or autoinflammatory disease or disorder.

The autoimmune disorder can include, but is not limited to, Addison's disease, alopecia areata, antiphospholipid antibody syndrome (aPL), autoimmune hepatitis, celiac disease-sprue (gluten-sensitive enteropathy), dermatomyositis, Graves' disease, Guillain-Barre syndrome, Hashimoto's disease, hemolytic anemia, idiopathic thrombocytopenic purpura, inflammatory bowel disease (IBD), inflammatory myopathies, multiple sclerosis, myasthenia gravis, pernicious anemia, primary biliary cirrhosis, psoriasis, reactive arthritis, rheumatoid arthritis, scleroderma, Sjögren's syndrome, systemic lupus erythematosus, Type I diabetes and vitiligo.

The autoinflammatory disorder can include, but is not limited to, familial Mediterranean fever (FMF), neonatal onset multisystem inflammatory disease (NOMID), tumor necrosis factor (TNF), receptor-associated periodic syndrome (TRAPS), deficiency of the interleukin-1 receptor antagonist (DIRA) and Behçet's disease.

A method is provided of treating a patient having chronic or acute myeloid leukemia (CML/AML), acute lymphoblastic leukemia (ALL), mantle cell lymphoma (MCL), or large B-cell lymphoma, comprising administering to the patient, an effective amount of the compound.

A method is provided of treating bone marrow for human bone marrow transplant treatment of leukemia in a patient, comprising treating bone marrow with the compound prior to reintroducing bone marrow into the patient.

A method is provided of inhibiting angiogenesis in a patient in need thereof, comprising administering to the patient, an effective amount of the compound.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated, enlarged, exploded, or incomplete to facilitate an understanding of the invention.

FIG. 1. Oxidation products formed from the reaction of parthenolide (1) with engineered P450 variant FL#62: 1,10-epoxy-parthenolide (2), 9(S)-hydroxy-parthenolide (3), 14-hydroxy-parthenolide (4).

FIG. 2. Synthesis and chemical structures of C9-substituted derivatives of parthenolide prepared according to the methods provided herein.

FIG. 3. Synthesis and chemical structures of 14-substituted derivatives of parthenolide prepared according to the methods provided herein.

FIG. 4. Chemical structures of C9-substituted derivatives of parthenolide prepared according to the methods provided herein.

FIG. 5. Chemical structures of C14-substituted derivatives of parthenolide prepared according to the methods provided herein.

FIG. 6. Synthesis of 9,13-disubstituted parthenolide derivatives (and salts thereof) according to the methods provided herein.

FIG. 7. Antileukemic activity of parthenolide and C9-substituted parthenolide derivatives as measured based on reduction of cell viability upon incubation with two different primary human acute myelogenous leukemia (AML) specimens (AML123009 and AML100510).

FIG. 8. C Antileukemic activity of parthenolide and C14-substituted parthenolide derivatives as measured based on reduction of cell viability upon incubation with two different primary human acute myelogenous leukemia (AML) specimens (AML123009 and AML100510).

FIG. 9. Cytotoxicity of representative parthenolide analogs against (A) total and (B) primitive (CD34+CD38−) normal human bone marrow cells. PTL is included for comparison.

FIG. 10. Antileukemic activity of representative C9- and C14-substituted parthenolide derivatives as measured based on reduction of cell viability upon incubation of M9-ENL1 cells with the compounds at varying concentrations. PTL is included as reference.

FIG. 11. In vitro cytotoxicity of selected C9- and C14-substituted parthenolide analogs as measured based on reduction of cell viability upon incubation of normal hematopoietic cells (cord blood cells) and progenitor cells (CD34⁺) with the compounds at varying concentrations. PTL is included as reference.

FIG. 12. Anticancer activity of representative C9- and C14-substituted parthenolide derivatives as measured based on reduction of cell viability upon incubation with various cells lines of human mantle cell lymphoma (MCL). PTL is included as reference.

FIG. 13. Anticancer activity of representative C9- and C14-substituted parthenolide derivatives as measured based on reduction of cell viability upon incubation with Diffuse Large B-cell Lymphoma cells (DLBCL) and primary human Chronic Lymphocytic Leukemia (CLL), and Acute Lymphoblastic Leukemia (ALL) specimens. PTL is included as reference.

FIGS. 14-1-14-9. SEQ ID NOS: 1-20 disclosed herein.

### 5. DETAILED DESCRIPTION OF THE INVENTION

Methods are provided for the generation of parthenolide derivatives functionalized at carbon atoms C9 and C14. The invention is based on the discovery that certain natural cytochrome P450 enzymes, and engineered variants of these enzymes, can be used to carry out the hydroxylation of these sites in parthenolide. According to the methods disclosed herein, these P450-catalyzed C—H hydroxylation reactions can be coupled to chemical interconversion of the enzymatically introduced hydroxyl group in order to install a broad range of functionalities at these otherwise unreactive sites of the molecule. As further demonstrated herein, the methods provided herein can also be applied to enable the production of bifunctionalized parthenolide derivatives, which in addition to modifications at the level of carbon atom C9 or C14, are also functionalized at the level of carbon atom C13.

While currently available methods have permitted the preparation of C13-modified PTL derivatives, the methods provided herein enable the modification of additional positions in the parthenolide scaffold. These methods can be useful to functionalize the C9 and C14 positions, optionally in conjunction with functionalization at the C13 position, to generate next-generation parthenolide derivatives with unexpectedly improved pharmacological properties.

Attempts to carry out the oxidation of parthenolide have been reported in the past, these approaches relying on the use of oxidizing microbial strains such as Streptomyces fulvissimus, Rhizopus nigricans, and Rhodotorula rubra (Galal, Ibrahim et al. 1999). However, these biotransformations have resulted in the isolation of multiple oxidation products of 11,13-dehydroparthenolide, which lacks the α-methylene-γ-lactone moiety essential for biological activity. In addition, the biological catalyst(s) responsible for parthenolide oxidation in these organisms were not identified or characterized.

Prior to this disclosure, the utility of cytochrome P450 enzymes and engineered variants thereof, for parthenolide oxyfunctionalization was unknown. The inventors discovered that engineered variants of natural cytochrome P450 monooxygenase enzymes can be exploited for the purpose of hydroxylating aliphatic positions in the parthenolide carbocyclic backbone (i.e. position C9 and C14) with high efficiency (i.e. high turnover numbers) and, in some cases, with excellent degrees of regio- and stereoselectivity, while preserving the integrity of critical functionalities in the molecule, such as the α-methylene-γ-lactone moiety and the 4,5-epoxide group.

The synthesis of C9- or C14-functionalized derivatives of parthenolide has never been described before. The present invention provides methods to generate derivatives of this type via a two-step chemoenzymatic strategy, in which parthenolide is first hydroxylated to generate 9-hydroxy-parthenolide or 14-hydroxy-parthenolide by means of one or more P450 monooxygenase enzyme(s). These hydroxylated derivatives can be isolated (e.g., via chromatography or extraction) and then subjected to chemical reaction conditions suitable for converting the enzymatically installed hydroxyl group (—OH) into a different functional group, such as, for example, a halogen, an ether group, a thioether group, an acyloxy group, an amide group, or an amino group. Several reagents and reaction conditions are known in the art to perform the chemical interconversion of an hydroxyl group (—OH), including reagents and reaction conditions for alkylation, acylation, deoxohalogenation, and nucleophilic substitution of an hydroxyl group (—OH).

Furthermore, we demonstrate that using the methods provided herein it is also possible to first generate 9- or 14-substituted parthenolide derivatives chemoenzymatically and then use these compounds as intermediates to synthesize doubly substituted parthenolide derivatives (i.e. 9,13-disubstituted derivatives, 14,13-disubstituted derivatives), in which the C13 position is also modified. Within this aspect of the invention, previously reported methods that are suitable for the functionalization of the C13 site in parthenolide (Guzman, Rossi et al. 2006; Hwang, Chang et al. 2006; Nasim and Crooks 2008; Han, Barrios et al. 2009; Neelakantan, Nasim et al. 2009; Woods, Mo et al. 2011) (See also Crooks et. al, U.S. Pat. No. 7,312,242; U.S. Pat. No. 7,678,904; U.S. Pat. No. 8,124,652) can be applied, as long as the reaction conditions involved in these processes are compatible with the functional group(s) contained within the substituent preinstalled in position C9 or C14.

For clarity of disclosure, and not by way of limitation, the detailed description of the invention is divided into the subsections set forth below.

### 5.1 Definitions

The term “functional group” as used herein refers to a contiguous group of atoms that, together, may undergo a chemical reaction under certain reaction conditions. Examples of functional groups are, among many others, —OH, —NH₂, —SH, —(C═O)—, —N₃, —C≡CH.

The term “aliphatic” or “aliphatic group” as used herein means a straight or branched C₁₋₁₅ hydrocarbon chain that is completely saturated or that contains one or more units of unsaturation, or a monocyclic C₃₋₈ hydrocarbon, or bicyclic C₈₋₁₂ hydrocarbon that is completely saturated or that contains one or more units of unsaturation, but which is not aromatic (also referred to herein as “cycloalkyl”). For example, suitable aliphatic groups include, but are not limited to, linear or branched alkyl, alkenyl, alkynyl groups or hybrids thereof such as (cycloalkyl)alkyl, (cycloalkenyl)alkyl, or (cycloalkynyl)alkyl. The alkyl, alkenyl, or alkynyl group may be linear, branched, or cyclic and may contain up to 15, preferably up to 8, and most preferably up to 5 carbon atoms. Preferred alkyl groups include methyl, ethyl, propyl, cyclopropyl, butyl, cyclobutyl, pentyl, and cyclopentyl groups. Preferred alkenyl groups include propenyl, butenyl, and pentenyl groups. Preferred alkynyl groups include propynyl, butynyl, and pentynyl groups.

The term “aryl” and “aryl group” as used herein refers to an aromatic substituent containing a single aromatic or multiple aromatic rings that are fused together, directly linked, or indirectly linked (such as linked through a methylene or an ethylene moiety). A aryl group may contain from 5 to 24 carbon atoms, preferably 5 to 18 carbon atoms, and most preferably 5 to 14 carbon atoms.

The terms “heteroatom” means nitrogen, oxygen, or sulphur, and includes any oxidized forms of nitrogen and sulfur, and the quaternized form of any basic nitrogen. Heteroatom further include Se, Si, and P.

The term “heteroaryl” as used herein refer to an aryl group in which at least one carbon atom is replaced with a heteroatom. Preferably, a heteroaryl group is a 5- to 18-membered, particularly a 5- to 14-membered, and especially a 5- to 10-membered aromatic ring system containing at least one heteroatom selected from the group consisting of oxygen, sulphur, and nitrogen atoms. Preferred heteroaryl groups include pyridyl, pyrrolyl, furyl, thienyl, indolyl, isoindolyl, indolizinyl, imidazolyl, pyridonyl, pyrimidyl, pyrazinyl, oxazolyl, thiazolyl, purinyl, quinolinyl, isoquinolinyl, benzofuranyl, and benzoxazolyl groups.

A heterocyclic group may be any monocyclic or polycyclic ring system which contains at least one heteroatom and may be unsaturated or partially or fully saturated. The term “heterocyclic” thus includes heteroaryl groups as defined above as well as non-aromatic heterocyclic groups. Preferably, a heterocyclic group is a 3- to 18-membered, particularly a 3- to 14-membered, and especially a 3- to 10-membered, ring system containing at least one heteroatom selected from the group consisting of oxygen, sulphur, and nitrogen atoms. Preferred heterocyclic groups include the specific heteroaryl groups listed above as well as pyranyl, piperidinyl, pyrrolidinyl, dioaxanyl, piperazinyl, morpholinyl, thiomorpholinyl, morpholinosulfonyl, tetrahydroisoquinolinyl, and tetrahydrofuranyl groups.

A halogen atom may be a fluorine, chlorine, bromine, or a iodine atom.

By “optionally substituted”, it is intended that in the any of the chemical groups listed above (e.g., alkyl, alkenyl, alkynyl, heteroalkyl, heteroalkenyl, heteroalkynyl, aryl, heteroaryl, heterocyclic, triazolyl groups), one or more hydrogen atoms are optionally replaced with an atom or chemical group other than hydrogen. Specific examples of such substituents include, without limitation, halogen atoms, hydroxyl (—OH), sulfhydryl (—SH), substituted sulfhydryl, carbonyl (—CO—), carboxy (—COOH), amino (—NH₂), nitro (—NO₂), sulfo (—SO₂—OH), cyano (—C≡N), thiocyanato (—S—C≡N), phosphono (—P(O)OH₂), alkyl, alkenyl, alkynyl, heteroalkyl, heteroalkenyl, heteroalkynyl, aryl, heteroaryl, heterocyclic, alkylthiol, alkyloxy, alkylamino, arylthiol, aryloxy, or arylamino groups. Where “optionally substituted” modifies a series of groups separated by commas (e.g., “optionally substituted A, B, or C”; or “A, B, or C optionally substituted with”), it is intended that each of the groups (e.g., A, B, or C) is optionally substituted.

The term “contact” as used herein with reference to interactions of chemical units indicates that the chemical units are at a distance that allows short range non-covalent interactions (such as Van der Waals forces, hydrogen bonding, hydrophobic interactions, electrostatic interactions, dipole-dipole interactions) to dominate the interaction of the chemical units. For example, when a protein is ‘contacted’ with a chemical species, the protein is allowed to interact with the chemical species so that a reaction between the protein and the chemical species can occur.

The term “polypeptide”, “protein”, and “enzyme” as used herein refers to any chain of two or more amino acids bonded in sequence, regardless of length or post-translational modification. According to their common use in the art, the term “protein” refers to any polypeptide consisting of more than 50 amino acid residues. These definitions are however not intended to be limiting.

In general, the term “mutant” or “variant” as used herein with reference to a molecule such as polynucleotide or polypeptide, indicates that such molecule has been mutated from the molecule as it exists in nature. In particular, the term “mutate” and “mutation” as used herein indicates any modification of a nucleic acid and/or polypeptide which results in an altered nucleic acid or polypeptide. Mutations include any process or mechanism resulting in a mutant protein, enzyme, polynucleotide, or gene. A mutation can occur in a polynucleotide or gene sequence, by point mutations, deletions, or insertions of single or multiple nucleotide residues. A mutation in a polynucleotide includes mutations arising within a protein-encoding region of a gene as well as mutations in regions outside of a protein-encoding sequence, such as, but not limited to, regulatory or promoter sequences. A mutation in a coding polynucleotide such as a gene can be “silent”, i.e., not reflected in an amino acid alteration upon expression, leading to a “sequence-conservative” variant of the gene. A mutation in a polypeptide includes but is not limited to mutation in the polypeptide sequence and mutation resulting in a modified amino acid. Non-limiting examples of a modified amino acid include a glycosylated amino acid, a sulfated amino acid, a prenylated (e.g., famesylated, geranylgeranylated) amino acid, an acetylated amino acid, an acylated amino acid, a PEGylated amino acid, a biotinylated amino acid, a carboxylated amino acid, a phosphorylated amino acid, and the like.

The term “engineer” or “engineered” refers to any manipulation of a molecule that result in a detectable change in the molecule, wherein the manipulation includes but is not limited to inserting a polynucleotide and/or polypeptide heterologous to the cell and mutating a polynucleotide and/or polypeptide native to the cell.

The term “polynucleotide molecule” as used herein refers to any chain of two or more nucleotides bonded in sequence. For example, a nucleic acid molecule can be a DNA or a RNA.

The terms “vector” and “vector construct” as used herein refer to a vehicle by which a DNA or RNA sequence (e.g., a foreign gene) can be introduced into a host cell, so as to transform the host and promote expression (e.g., transcription and translation) of the introduced sequence. A common type of vector is a “plasmid”, which generally is a self-contained molecule of double-stranded DNA that can be readily accept additional (foreign) DNA and which can readily introduced into a suitable host cell. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. The terms “express” and “expression” refer to allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be the to be “expressed” by the cell. A polynucleotide or polypeptide is expressed recombinantly, for example, when it is expressed or produced in a foreign host cell under the control of a foreign or native promoter, or in a native host cell under the control of a foreign promoter.

The term “fused” as used herein means being connected through one or more covalent bonds. The term “bound” as used herein means being connected through non-covalent interactions. Examples of non-covalent interactions are van der Waals, hydrogen bond, electrostatic, and hydrophobic interactions. The term “tethered” as used herein means being connected through covalent or non-covalent interactions. Thus, a “polypeptide tethered to a solid support” refers to a polypeptide that is connected to a solid support (e.g., surface, resin bead) either via non-covalent interactions or through covalent bonds.

### 5.2 P450 Monooxygenase Enzymes

The present invention provides cytochrome P450 polypeptides having the capability to oxidize parthenolide, wherein the cytochrome P450 polypeptide comprises an amino acid sequence having at least 60% sequence identity to SEQ. ID NO:1, SEQ. ID NO:2, or SEQ. ID NO:3 over a region of at least about 100, 200, 300, 400, 500, 1000, or more residues.

In some embodiments, the capability to oxidize parthenolide corresponds to the capability of the cytochrome P450 polypeptide to hydroxylate a C—H bond attached to the carbon atom C9 in parthenolide, where the resulting hydroxylated product has predominantly (S) or (R) stereochemistry at the hydroxylation site (C9) according to the stereoselectivity of the enzyme. In other embodiments, such capability corresponds to the capability of the cytochrome P450 polypeptide to hydroxylate a C—H bond attached to the carbon atom C14 in parthenolide.

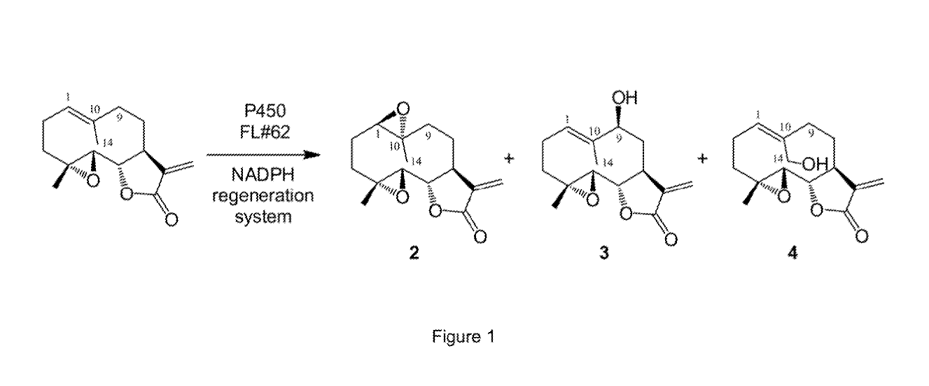

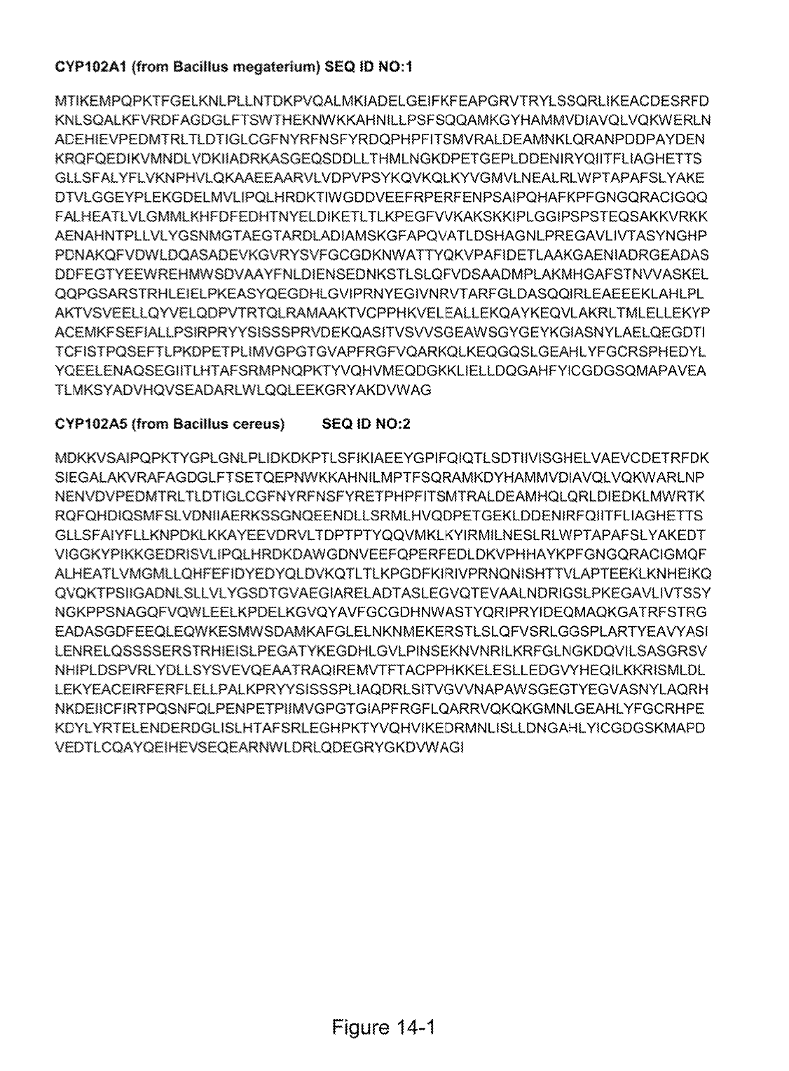

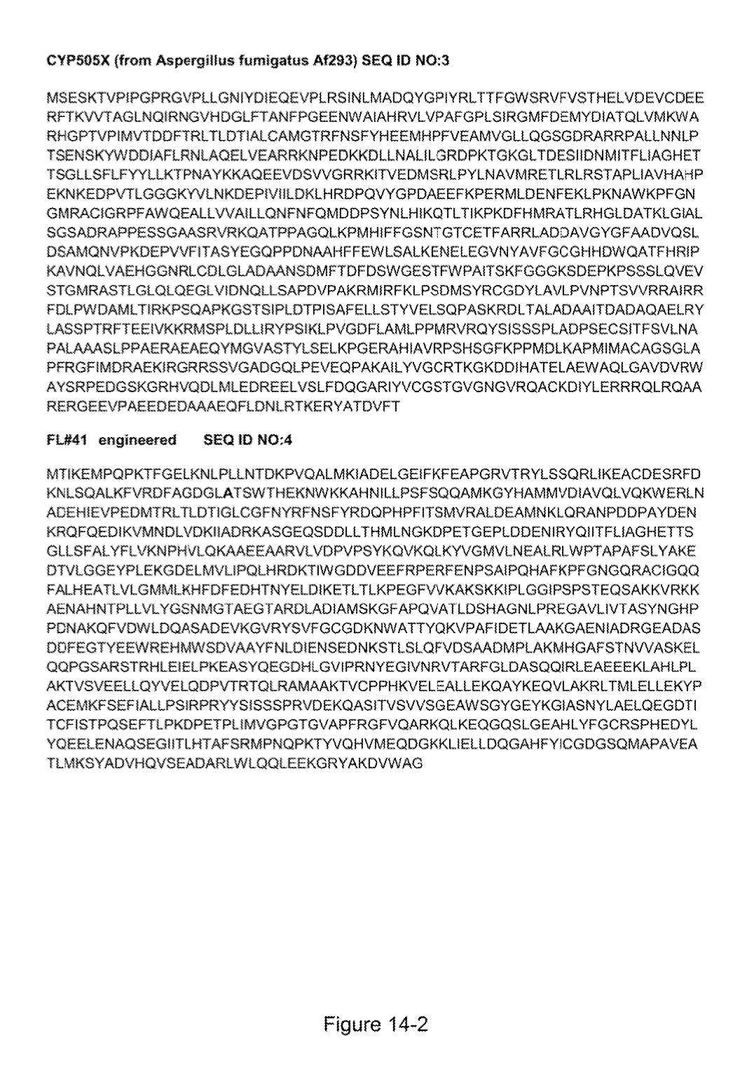

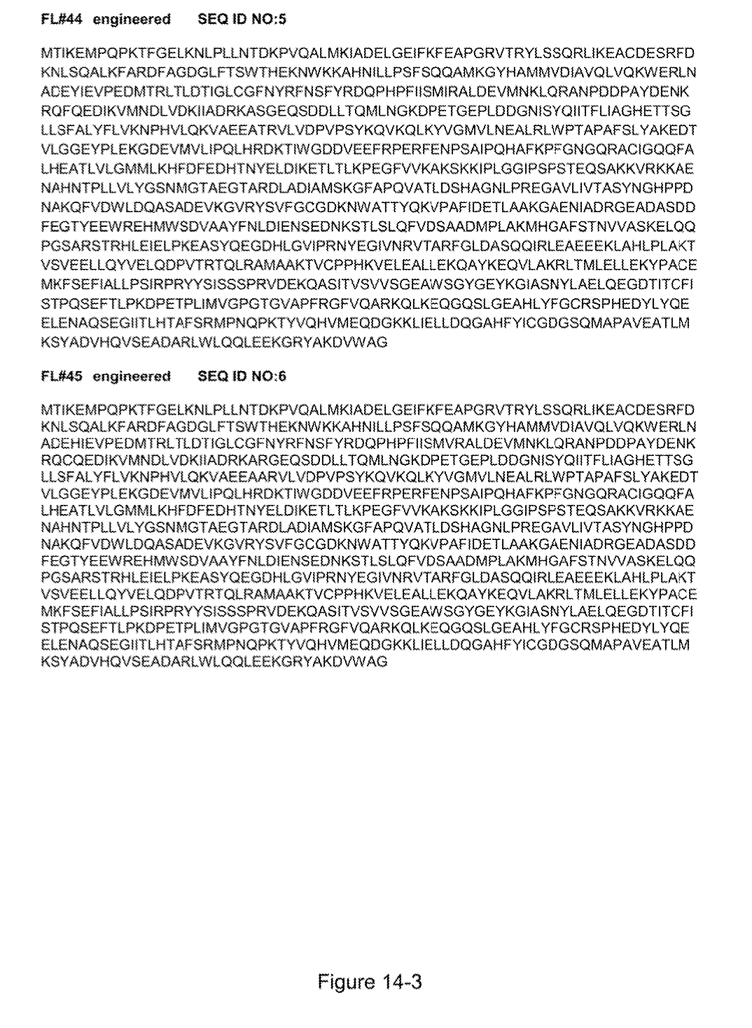

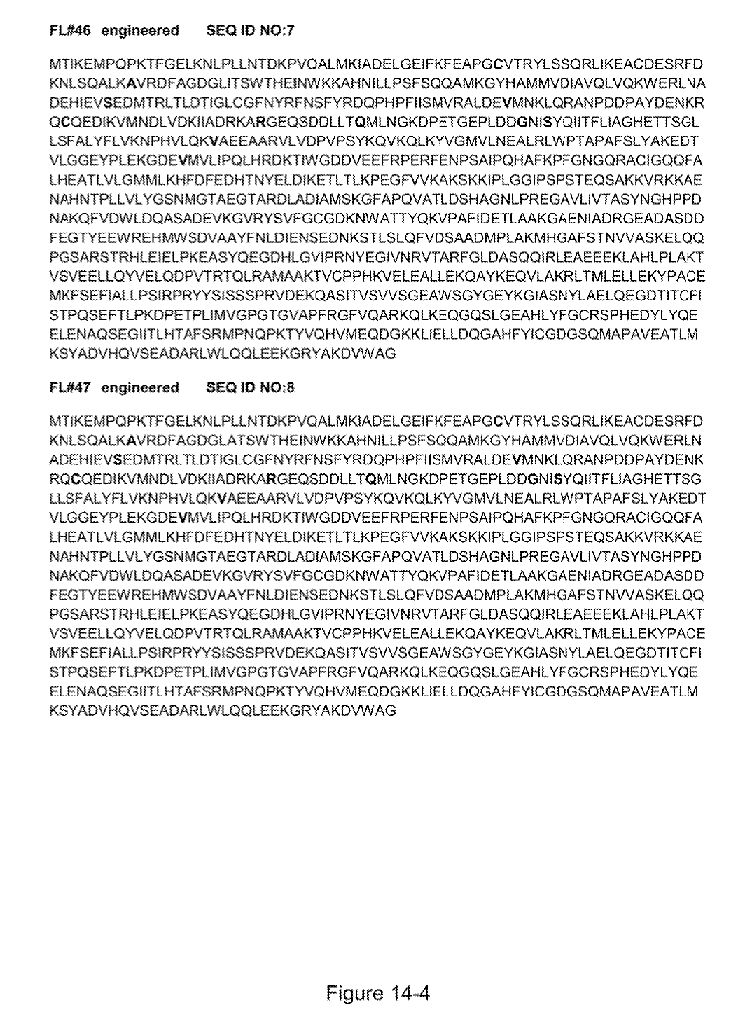

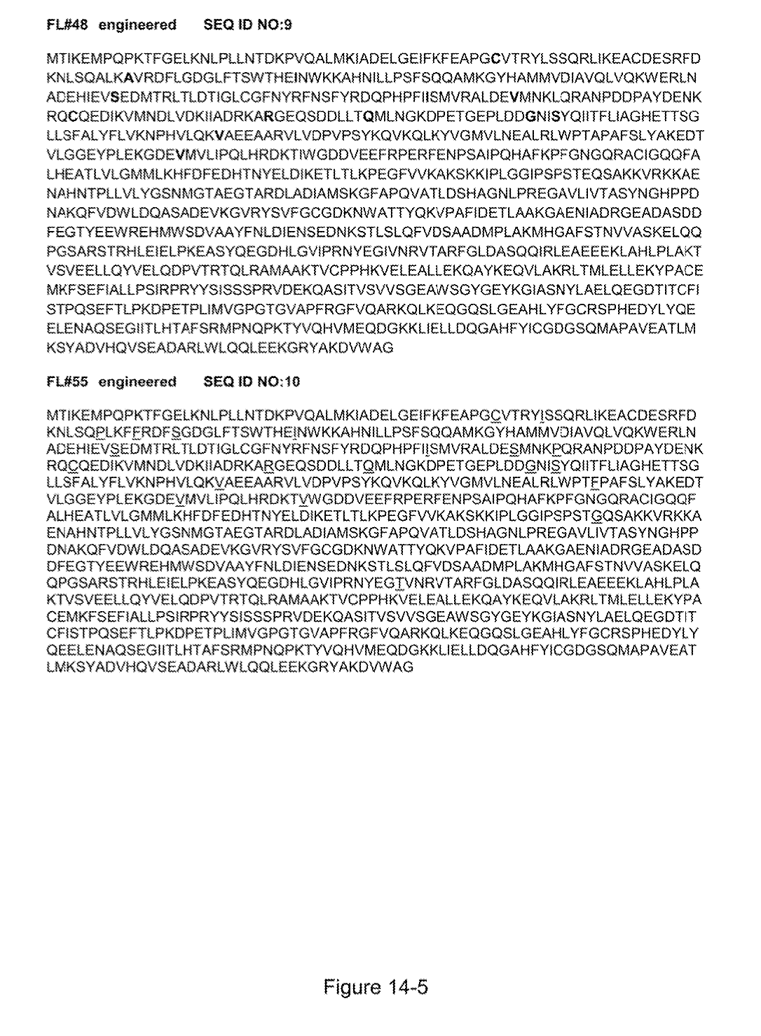

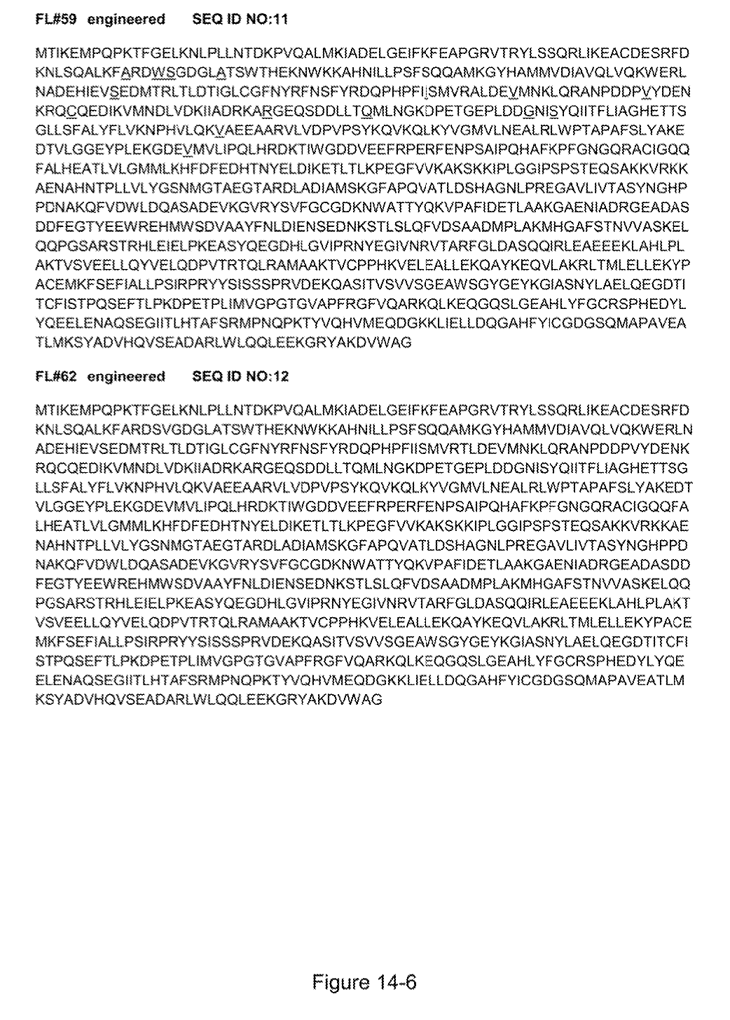

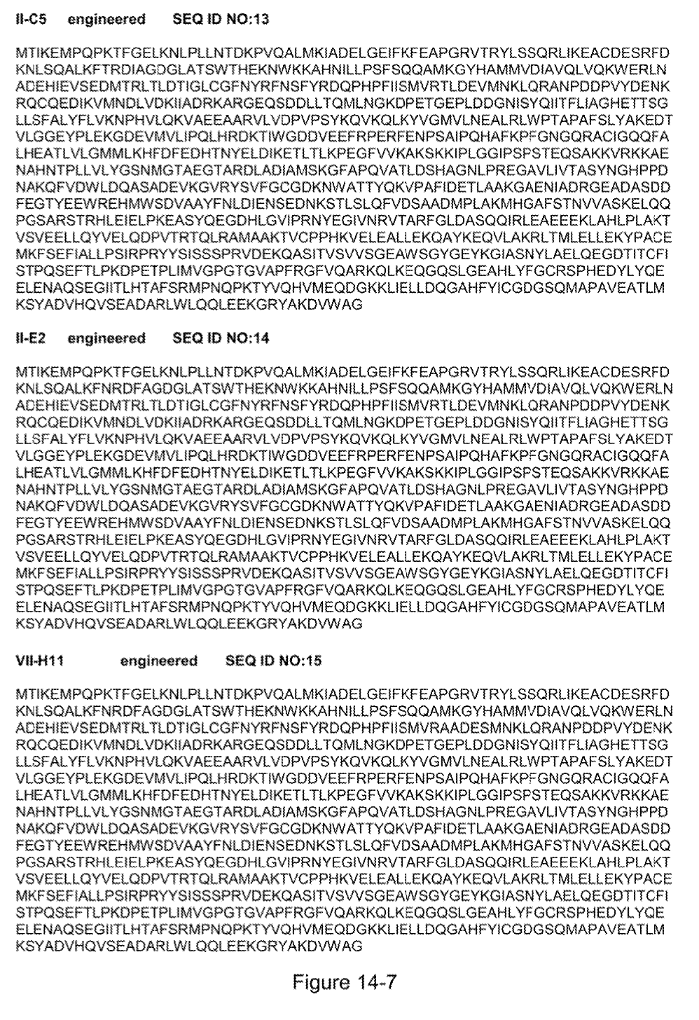

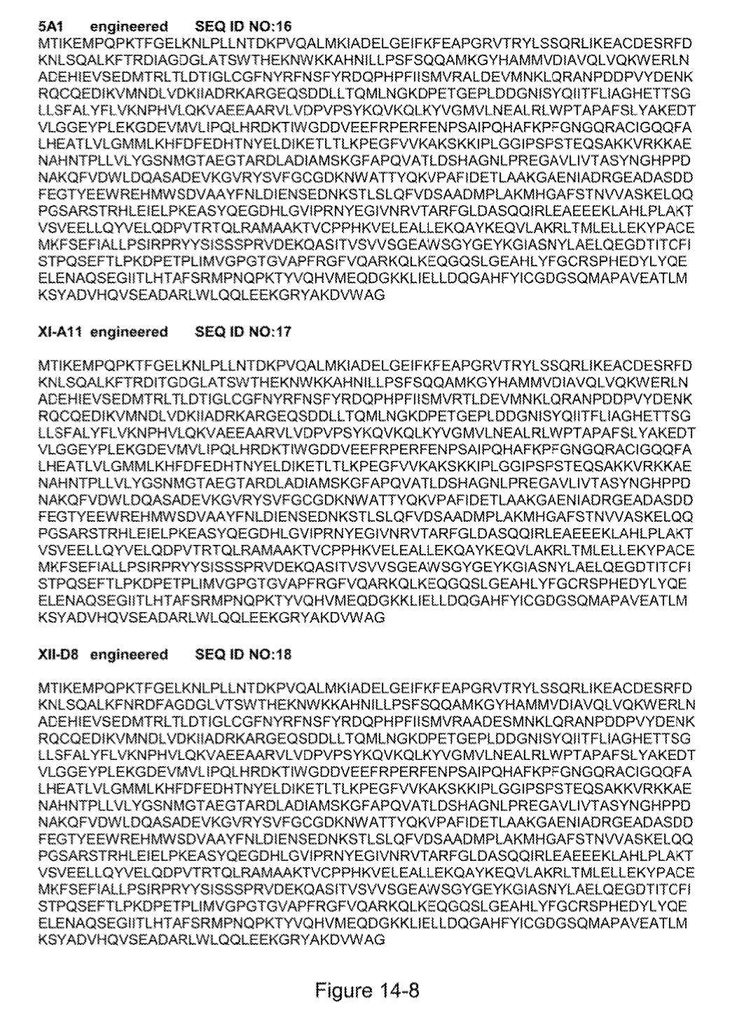

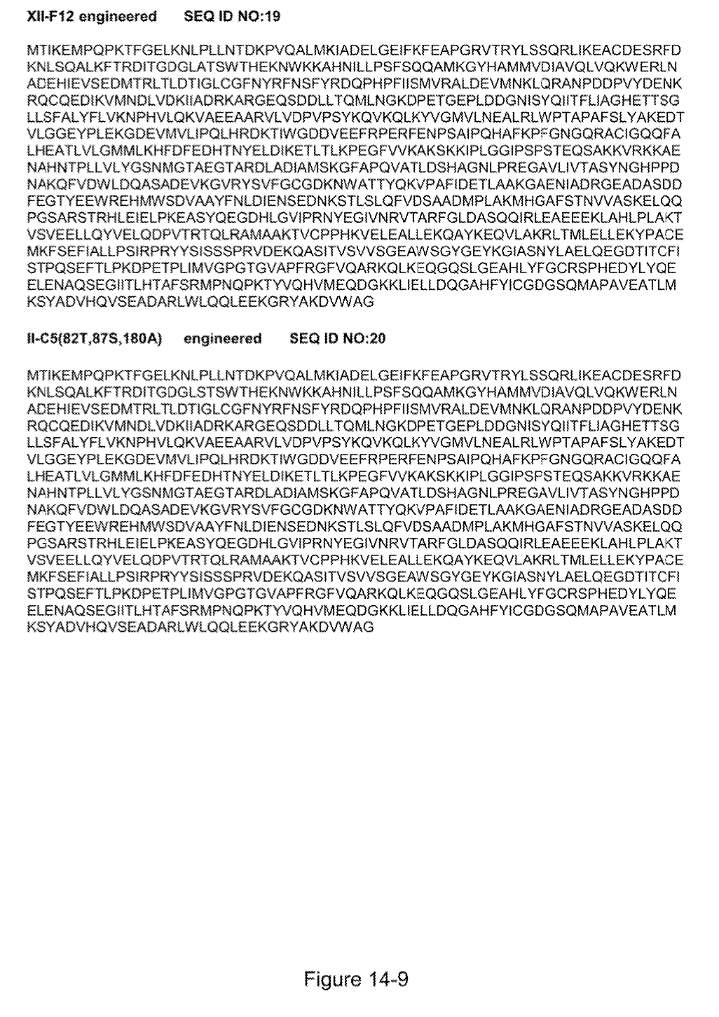

Cytochrome P450 polypeptides are provided that are capable of hydroxylating a C—H bond at position 9, position 14, or both, in parthenolide, and which have an improved property compared with a reference enzyme, such as the naturally occurring enzymes from which they were derived, the naturally occurring enzymes being CYP102A1 from Bacillus megaterium (SEQ ID NO: 1), CYP102A5 from Bacillus cereus (SEQ ID NO: 2), or CYP505X from Aspergillus fumigatus (SEQ ID NO: 3), or when compared with other engineered cytochrome P450 enzymes, such as the polypeptide of SEQ ID NO: 4 (see FIGS. 14-1-14-9).

In the characterization of the cytochrome P450 enzymes disclosed herein, the polypeptides can be described in reference to the amino acid sequence of a naturally occurring cytochrome P450 enzyme or another engineered cytochrome P450 enzyme. As such, the amino acid residue is determined in the cytochrome P450 enzymes beginning from the initiating methionine (M) residue (i.e., M represent residue position 1), although it will be understood that this initiating methionine residue may be removed by biological processing machinery such as in a host cell or in vitro translation system, to generate a mature protein lacking the initiating methionine residue. The amino acid residue position at which a particular amino acid or amino acid change is present is sometimes described herein as “Xn”, or “position n”, where n refers to the residue position.

As described above, the cytochrome P450 enzymes provided herein are characterized by an improved enzyme property as compared to the naturally occurring parent enzyme or another engineered cytochrome P450 enzyme. Changes to enzyme properties can include, among others, improvements in enzymatic activity, regioselectivity, stereoselectivity, and/or reduced substrate or product inhibition. In the embodiments herein, the altered properties are based on engineered cytochrome P450 polypeptides having residue differences at specific residue positions as compared to a reference sequence of a naturally occurring cytochrome P450 enzyme, such as CYP102A1 (SEQ ID NO: 1), CYP102A5 (SEQ ID NO: 2), or CYP505X (SEQ ID NO: 3), or as compared to another engineered cytochrome P450 enzyme, such as the polypeptide of SEQ ID NO: 4.

In some embodiments, the P450 monoxygenase is an engineered variant of CYP102A1 (SEQ ID NO: 1), the variant comprising an amino acid change at one or more of the following positions of SEQ ID NO: 1: X26, X27, X43, X48, X52, X53, X73, X75, X76, X79, X82, X83, X88, X89, X95, X97, X143, X146, X176, X181, X182, X185, X189, X198, X206, X226, X227, X237, X253, X256, X261, X264, X265, X268, X269, X291, X320, X331, X329, X330, X354, X355, X367, X394, X435, X436, X444, X446, X438, and X439.

In some embodiments, the P450 monoxygenase is an engineered variant of CYP102A5 (SEQ ID NO: 2), the variant comprising an amino acid change at one or more of the following amino acid positions of SEQ ID NO:2: X28, X29, X45, X50, X54, X55, X75, X77, X78, X81, X83, X85, X90, X91, X97, X99, X145, X148, X178, X183, X184, X187, X191, X200, X208, X228, X229, X240, X256, X259, X264, X267, X268, X271, X272, X294, X323, X334, X332, X333, X358, X359, X371, X398, X439, X440, X448, X440, X442, and X443.

In some embodiments, the P450 monoxygenase is an engineered variant of CYP505X (SEQ ID NO: 3), the variant comprising an amino acid change at one or more of the following amino acid positions of SEQ ID NO:3: X29, X30, X46, X51, X55, X56, X76, X78, X79, X82, X85, X86, X91, X92, X99, X101, X147, X151, X180, X185, X186, X189, X193, X202, X210, X230, X231, X241, X257, X260, X265, X268, X269, X272, X273, X295, X324, X335, X333, X334, X365, X366, X378, X405, X446, X447, X455, X457, X449, and X450.

In some embodiments, the cytochrome P450 polypeptides can have additionally one or more residue differences at residue positions not specified by an X above as compared to the sequence SEQ ID NO: 1, SEQ ID NO: 2, or SEQ ID NO: 3. In some embodiments, the differences can be 1-2, 1-5, 1-10, 1-20, 1-30, 1-40, 1-50, 1-75, 1-100, 1-150, or 1-200 residue differences at other amino acid residue positions not defined by X above.

In some embodiments, the cytochrome P450 polypeptides can have additionally one or more residue differences at residue positions not specified by an X above and located within the “heme domain” of the enzyme, as compared to the sequence SEQ ID NO: 1, SEQ ID NO: 2, or SEQ ID NO: 3. In some embodiments, the differences can be 1-2, 1-5, 1-10, 1-20, 1-30, 1-40, 1-50, 1-75, 1-100, 1-150, or 1-200 residue differences at other amino acid residue positions not defined by X above and located within the “heme domain” of the enzyme.

In some embodiments, the engineered cytochrome P450 polypeptides having one or more of the improved enzyme properties described herein, can comprise an amino acid sequence that is at least 60%, 70%, 80%, 85%, 90%, 95%, 99% or more identical to the sequence SEQ ID NO: 1, SEQ ID NO: 2, or SEQ ID NO: 3.

In some embodiments, the engineered cytochrome P450 polypeptides having one or more of the improved enzyme properties described herein, can comprise an amino acid sequence encompassing its heme domain which is at least 60%, 70%, 80%, 85%, 90%, 95%, 99% or more identical to the amino acid sequence encompassing the first 500 amino acids in the sequence SEQ ID NO: 1, SEQ ID NO: 2, or SEQ ID NO: 3 (i.e. residue 1 to residue 500 in these reference sequences).

In some embodiments, the improved cytochrome P450 polypeptide can comprise an amino acid sequence that is at least 60%, 70%, 80%, 85%, 90%, 95%, 99% or more identical to a sequence corresponding to SEQ ID NO: 4, 5, 6, 7, 8, 9, 10, 11, 12, 13, 14, 15, 16, 17, 18, 19, or 20 (see FIGS. 14-1-14-9).

In some embodiments, the improved cytochrome P450 polypeptide can comprise an amino acid sequence encompassing its heme domain that is at least 60%, 70%, 80%, 85%, 90%, 95%, 99% or more identical to the sequence encompassing the first 500 amino acids in SEQ ID NO: 4, 5, 6, 7, 8, 9, 10, 11, 12, 13, 14, 15, 16, 17, 18, 19, or 20.

In some embodiments, the improved cytochrome P450 polypeptide comprises an amino acid sequence corresponding to the sequence of SEQ ID NO: 4, 5, 6, 7, 8, 9, 10, 11, 12, 13, 14, 15, 16, 17, 18, 19, or 20.

In some embodiments, the improved enzyme property of the engineered P450 polypeptide is with respect to its catalytic activity, coupling efficiency, regioselectivity and/or stereoselectivity.

The improvement in catalytic activity can be manifested by an increase in the number of total turnovers supported by the P450 polypeptide for parthenolide oxidation, as compared to the wild-type parental sequence (SEQ ID NO: 1, SEQ ID NO: 2, or SEQ ID NO: 3), or other reference sequences (e.g., SEQ ID NO: 4). In some embodiments, the cytochrome P450 polypeptides are capable of supporting a number of total turnovers that is at least 1.1-fold, 2-fold, 5-fold, 10-fold, 100-fold, 200-fold, 500-fold, or more higher than the number of total turnovers supported by its respective naturally occurring parental sequence SEQ ID NO: 1, SEQ ID NO: 2, or SEQ ID NO: 3.

The improvement in catalytic activity can be also manifested by an increase in the catalytic efficiency for the oxidation of a given substrate, this catalytic efficiency being conventionally defined by the k_{cat}/K_{M} ratio, where k_{cat} is the turnover number and K_{M} is the Michaelis-Menten constant, as compared to the wild-type parental sequence (SEQ ID NO: 1, SEQ ID NO: 2, or SEQ ID NO: 3), or other reference sequences (e.g., SEQ ID NO: 4). In some embodiments, the cytochrome P450 polypeptides exhibit a catalytic efficiency that is at least 1.1-fold, 2-fold, 5-fold, 10-fold, 100-fold, 200-fold, 500-fold, or more higher than the catalytic efficiency of its respective naturally occurring parental sequence SEQ ID NO: 1, SEQ ID NO: 2, or SEQ ID NO: 3.

In some embodiments, the engineered P450 polypeptides having improved catalytic activity on parthenolide comprise an amino acid sequence corresponding to SEQ ID NO: 4, 5, 6, 7, 8, 9, 10, 11, 12, 13, 14, 15, 16, 17, 18, 19, or 20.

The improvement in coupling efficiency can be manifested by an increase in the ratio between the moles of oxidation product formed by the enzyme per unit of time and the moles of cofactor molecules (e.g., NAD(P)H) consumed by the enzyme per unit of time. In some embodiments, the cytochrome P450 polypeptides are capable of oxidizing parthenolide with a coupling efficiency that is at least 1%, 2%, 5%, 10%, 20%, 30%, 40%, 50%, 75%, 90%, 98%, 99% or more higher than the coupling efficiency of its respective naturally occurring parental sequence SEQ ID NO: 1, SEQ ID NO: 2, or SEQ ID NO: 3 or the reference sequence SEQ ID NO: 4.

In some embodiments, the engineered P450 polypeptides having improved coupling efficiency comprise an amino acid sequence corresponding to SEQ ID NO: 4, 5, 6, 7, 8, 9, 10, 11, 12, 13, 14, 15, 16, 17, 18, 19, or 20.

The improvement in regioselectivity can be manifested by an increase in the selectivity by which a particular C—H bond in parthenolide is oxidized by action of the engineered cytochrome P450 polypeptide over the other C—H bonds occurring in the molecule, as compared to the wild-type parental sequence (SEQ ID NO: 1, SEQ ID NO: 2, or SEQ ID NO: 3), or other reference sequences (e.g., SEQ ID NO: 4). In some embodiments, the cytochrome P450 polypeptides are capable of oxidizing parthenolide with a regioselectivity that is at least 1%, 2%, 5%, 10%, 20%, 30%, 40%, 50%, 75%, 90%, 98%, 99% or more higher than that exhibited by its respective wild-type parental sequence SEQ ID NO: 1, SEQ ID NO: 2, or SEQ ID NO: 3 or the reference sequence SEQ ID NO: 4 toward the oxidation of the same C—H bond in parthenolide.

In some embodiments, the engineered P450 polypeptides having improved regioselectivity toward oxidation of carbon atom C9 in parthenolide, as compared to the sequence SEQ ID NO: 1, comprise an amino acid sequence corresponding to SEQ ID NO: 4, 9, 12, 13, 14, 16, 17, 19, or 20. In some embodiments, the engineered P450 polypeptides having improved regioselectivity toward oxidation of carbon atom C14 in parthenolide, as compared to the sequence SEQ ID NO: 1, comprise an amino acid sequence corresponding to SEQ ID NO: 5, 6, 7, 14, 15, or 18.

In some embodiments, the improvement in stereoselectivity can be manifested by an increase in the stereoselectivity by which a C—H bond in a prochiral carbon atom of parthenolide (e.g., C9) is oxidized by action of the engineered cytochrome P450 polypeptide as compared to the wild-type parental sequence (SEQ ID NO: 1, SEQ ID NO: 2, or SEQ ID NO: 3), or other reference sequences (e.g., SEQ ID NO: 4). The degree of stereoselectivity can be conventionally described in terms of stereomeric excess, that is in terms of enantiomeric excess (ee) or diasteromeric excess (de) depending on the nature of the substrate. In some embodiments, the improvement in stereoselectivity in the engineered cytochrome P450 polypeptide is with respect to producing the (S) stereoisomer of the hydroxylation product (i.e., stereoisomer in which the absolute configuration of the hydroxylation site is (S)). In some embodiments, such improvement in stereoselectivity is with respect to producing the (R) stereoisomer of the hydroxylation product. In some embodiments, the cytochrome P450 polypeptides are capable of oxidizing parthenolide with a (S)- or (R)-stereoselectivity (i.e. stereomeric excess) that is at least 1%, 2%, 5%, 10%, 20%, 30%, 40%, 50%, 75%, 90%, 98%, 99% or more higher than that exhibited by its respective wild-type parental sequence SEQ ID NO: 1, SEQ ID NO: 2, or SEQ ID NO: 3, or the reference sequence SEQ ID NO: 4, toward the oxidation of the same carbon atom in parthenolide.

In some embodiments, the engineered P450 polypeptides having improved stereoselectivity toward oxidation of carbon atom C9 in parthenolide comprise an amino acid sequence corresponding to SEQ ID NO: 4, 9, 12, 13, 14, 16, 17, 19, or 20.

The capability of the engineered cytochrome P450 polypeptides to oxidize parthenolide, also referred to herein as “substrate,” can be established according to methods well known in the art. Most typically, such capability can be established by contacting the substrate with the P450 monooxygenase under suitable reaction conditions in which the P450 monooxygenase is catalytically functional, and then determining the formation of an oxidized product of the substrate (e.g., hydroxylated product) by standard analytical methods such as, for example, thin-layer chromatography, HPLC, and/or LC-MS.

Various art-known methods can be applied for measuring the catalytic activity of the engineered cytochrome P450 polypeptide on parthenolide, also referred to herein as “substrate activity”. Such substrate activity can be measured by measuring the decrease of the amount of substrate, the accumulation of an oxygenation product derived from the substrate (e.g., hydroxylated product), or the accumulation of an oxidation byproduct generated during the enzymatic reaction (e.g., H₂O₂), after a given time after contacting the substrate with the P450 monooxygenase under suitable reaction conditions in which the P450 monooxygenase is catalytically functional. Other methods to measure the substrate activity include measuring the consumption of a cofactor (e.g., NADPH or NADH) or cosubstrate (O₂) utilized by the enzyme during the oxidation reaction. The choice of the method will vary depending on the specific application such as, for example, according to the nature of the substrate, the nature of the monooxygenase (e.g., its NAD(P)H cofactor specificity), and the number of the P450 monooxygenases that are to be evaluated. A person skilled in the art will be capable of selecting the most appropriate method in each case.

The substrate activity of engineered cytochrome P450 polypeptides can be measured and expressed in terms of number of catalytic turnovers, product formation rate, cofactor consumption rate, O₂ consumption rate, H₂O₂ consumption rate (e.g., for H₂O₂-dependent monooxygenases), and the like. Most conveniently, such substrate activity can be measured and expressed in terms of total turnover numbers (or TTN), which corresponds to the total number of catalytic turnovers supported by the P450 monooxygenase enzyme on this substrate.

In some embodiments, the engineered cytochrome P450 polypeptides disclosed herein are capable of supporting at least 1, 10, 50, 100, or more TTN in the oxidation of parthenolide.

The regio- and stereoselectivity of the engineered cytochrome P450 polypeptides for the oxidation of parthenolide can be measured by determining the relative distribution of oxidation products generated by the reaction between the substrate and the cytochrome P450 polypeptide using conventional analytical methods such as, for example, (chiral) normal phase liquid chromatography, (chiral) reverse-phase liquid chromatography, or (chiral) gas chromatography. In some instances, the oxidation products can be subjected to a chemical derivatization process to facilitate these analyses. For example, the hydroxylation products obtained from the reaction of the P450 polypeptide with parthenolide can be derivatized using an UV-active acid chloride (e.g., benzoyl chloride) prior to separation and quantification by HPLC.

In some embodiments, the engineered cytochrome P450 polypeptides disclosed herein are capable of hydroxylating a C—H bond connected to the C9 or C14 carbon atom in parthenolide with a regioselectivity of 1%, 5%, 10%, 25%, 50%, 75%, 90%, 95%, or higher.

In some embodiments, the P450 monooxygenase is a CYP102A1-derived variant selected from the group consisting of FL#41 (SEQ ID NO: 4), FL#44 (SEQ ID NO: 5), FL#45 (SEQ ID NO: 6), FL#46 (SEQ ID NO: 7), FL#47 (SEQ ID NO: 8), FL#48 (SEQ ID NO: 9), FL#55 (SEQ ID NO: 10), FL#59 (SEQ ID NO: 11), and FL#62 (SEQ ID NO: 12). These P450 monooxygenases were found to be capable of oxidizing parthenolide with varying catalytic activity (i.e., with varying numbers of total turnovers) and with varying degree of regio- and stereoselectivity. Wild-type CYP102A1 (SEQ ID NO: 1) exhibits moderate oxidation activity on parthenolide (TTN: 29), producing 1,10-epoxy-parthenolide as the only product. In contrast, FL#44 (SEQ ID NO: 5) is capable of oxidizing parthenolide with higher catalytic activity (493 TTN), producing 1,10-epoxy-parthenolide (2), 9-hydroxy-parthenolide (3), and 14-hydroxy-parthenolide (4) in 61:2:37 ratio. Compared to its naturally occurring parent enzyme CYP102A1 (SEQ ID NO: 1), FL#44 (SEQ ID NO: 5) carries the following amino acid changes: V79A, H139Y, T176I, V179I, A185V, H237Q, E253G, R256S, A291V, A296T, L354V. As another example, FL#48 (SEQ ID NO: 9) is capable of oxidizing parthenolide, producing 1,10-epoxy-parthenolide (2), 9-hydroxy-parthenolide (3), and 14-hydroxy-parthenolide (4) in 67:24:9 ratio, and supporting about 58 total turnovers. Compared to the its parent enzyme CYP102A1 (SEQ ID NO: 1), FL#44 (SEQ ID NO: 5) carries the following amino acid changes: R48C, V79A, A83L, K95I, P143S, T176I, A185V, F206C, S227R, H237Q, E253G, R256S, A291V, L354V. As another example, FL#62 (SEQ ID NO: 12) was found to be capable of hydroxylating parthenolide, producing 1,10-epoxy-parthenolide (2), 9-hydroxy-parthenolide (3), and 14-hydroxy-parthenolide (4) in 77:13:10 ratio, and supporting about 888 total turnovers. Compared to its parent enzyme CYP102A1 (SEQ ID NO: 1), FL#62 (SEQ ID NO: 8) carries the following amino acid changes: V79A, F82S, A83V, F88A, P143S, T176I, A181T, A185V, A198V, F206C, S227R, H237Q, E253G, R256S, A291V, L354V.

In some embodiments, the cytochrome P450 polypeptide is a FL#62-derived variant selected from the group consisting of II-C5 (SEQ ID NO: 13), II-E2 (SEQ ID NO: 14), VII-H11 (SEQ ID NO: 15), 5A1 (SEQ ID NO: 16), XI-A11 (SEQ ID NO: 17), XII-D8 (SEQ ID NO: 18), XII-F12 (SEQ ID NO: 19), and II-C5(82T,87S,180A) (SEQ ID NO: 20). These cytochrome P450 polypeptides were prepared by mutagenesis of FL#62 (SEQ ID NO: 12) at one or more of the residues selected from the group consisting of residue X26, X27, X43, X48, X52, X53, X73, X75, X76, X79, X82, X83, X88, X89, X95, X97, X143, X146, X176, X181, X182, X185, X189, X198, X206, X226, X227, X237, X253, X256, X261, X264, X265, X268, X269, X291, X320, X331, X329, X330, X354, X355, X367, X394, X435, X436, X444, X446, X438, and X439. These cytochrome P450 polypeptides exhibit improved catalytic activity and/or regio- and stereoselectivity toward the hydroxylation of parthenolide compared to the wild-type enzyme CYP102A1 (SEQ ID NO: 1) or to FL#62 (SEQ ID NO: 12). As an example, VII-H11 (SEQ ID NO: 15), which carries the amino acid mutations A88N, S82F, V83A, T181A, L182A, V185S compared to FL#62 (SEQ ID NO: 12), exhibits improved regioselectivity for C14 hydroxylation (79% vs. 10%). As another example, XI-A11 (SEQ ID NO: 17), which carries the amino acid mutations A79T, S82I, V83T compared to FL#62 (SEQ ID NO: 12), exhibits improved regioselectivity for C9 hydroxylation (69% vs. 13%).

In some embodiments, the improved engineered cytochrome P450 polypeptides comprise deletions of the engineered cytochrome P450 polypeptides disclosed herein. Accordingly, for each of the embodiment of the cytochrome P450 polypeptides provided herein, the deletions can comprise 1, 2, 5, 10, 50, 100 or more amino acids, as long as the functional activity and/or improved properties of the P450 polypeptide is maintained.

In some embodiments, the improved engineered cytochrome P450 polypeptides can comprise fragments of the engineered cytochrome P450 polypeptides disclosed herein. In some embodiments, the polypeptide fragments can be 50%, 60%, 70%, 80%, 90%, 95%, 98%, or 99% of the full-length cytochrome P450 polypeptide, such as the polypeptides of SEQ ID NO: 4 through 20.

In some embodiments, the improved engineered cytochrome P450 polypeptides can comprise only the heme domain of the engineered cytochrome P450 polypeptides disclosed herein. Typically, albeit not necessarily, such heme domain is encompassed by the first (i.e., N-terminal) 500 amino acid residues of the engineered cytochrome P450 polypeptides. The heme domain comprises the active site in which the substrate binds and is oxidized. The amino acid mutations comprised within the heme domain are therefore primarily responsible for the improved substrate recognition properties and/or regio- and stereoselectivity properties of the engineered cytochrome P450 polypeptides. The remainder of the polypeptide sequence comprises the reductase component of the enzyme (FMN/FAD diflavin-dependent reductase domain), whose role is to transfer electrons from a soluble cofactor (i.e., NADPH) to the heme domain to drive the catalytic cycle.

It is known in the art that the heme domain in catalytically self-sufficient cytochrome P450 enzymes such as CYP102A1, CYP102A5, and CYP505X can be covalently or non-covalently linked to a non-native electron-transfer system resulting in a functional, artificial P450 system. For example, the non-native electron-transfer system may be the reductase domain of a P450 enzyme from the same CYP subfamily (Landwehr, Carbone et al. 2007), the reductase domain of a P450 enzyme from a different CYP subfamily (e.g., RhF reductases) (Li, Podust et al. 2007), or the redox partners of a class I P450 system (e.g., ferrodoxin and ferrodoxin reductase)(Hirakawa and Nagamune 2010). Alternatively, the non-native electron-transfer system can be an electrode or light in combination or not with redox active compounds, which deliver one or more electrons to the P450 heme domain to drive catalysis. (Tran, Huynh et al. 2011) Alternatively, the non-native electron-transfer system can be a chemical reagent, such as H₂O₂ or an organic peroxide, which can react with the heme cofactor in the heme domain of the P450 polypeptide and drive catalysis through the peroxide shunt pathway, thereby serving as a source of both oxygen and electrons and bypassing the need for a reductase component. (Cirino and Arnold 2003; Otey, Landwehr et al. 2006)

Accordingly, in some embodiments, the improved engineered cytochrome P450 polypeptide or a fragment thereof (e.g., its heme domain), is comprised in an artificial P450 system, that is, a system that comprises the full-length cytochrome P450 polypeptide or a fragment thereof and an exogenous electron-transfer system, this exogenous electron-transfer system being one or more protein-based, chemical, or physical agents, which can deliver one or more electrons to the heme cofactor in the P450 polypeptide.

In some embodiments, the improved engineered cytochrome P450 polypeptides can comprise one or more non-natural amino acids. The non-natural amino acid can be present at one or more of the positions defined by “Xn” above for the purpose of modulating the enzyme properties of the polypeptide. Alternatively, the non-natural amino acid can be introduced in another position of the polypeptide sequence for the purpose, for example, of linking the P450 polypeptide to another protein, another biomolecule, or a solid support. Several methods are known in the art for introducing an unnatural amino acid into a polypeptide. These include the use of the amber stop codon suppression methods using engineered tRNA/aminoacyl-tRNA synthetase (AARS) pairs such as those derived from Methanococcus sp. and Metanosarcinasp. (Liu and Schultz 2010). Alternatively, natural or engineered frameshift suppressor tRNAs and their cognate aminoacyl-tRNA synthetases can also be used for the same purpose (Rodriguez, Lester et al. 2006; Neumann, Wang et al. 2010). Alternatively, an unnatural amino acid can be incorporated in a polypeptide using chemically (Dedkova, Fahmi et al. 2003) or enzymatically (Bessho, Hodgson et al. 2002) aminoacylated tRNA molecules and using a cell-free protein expression system in the presence of the aminoacylated tRNA molecules (Kourouklis, Murakami et al. 2005; Murakami, Ohta et al. 2006). Examples of non-natural amino acids include but are not limited to, para-acetyl-phenylalanine, meta-acetyl-phenylalanine, para-butyl-1,3-dione-phenylalanine, O-allyl-tyrosine, O-propargyl-tyrosine, para-azido-phenylalanine, para-borono-phenylalanine, para-bromo-phenylalanine, para-iodo-phenylalanine, 3-iodo-tyrosine, para-benzoyl-phenylalanine, para-benzoyl-phenylalanine, ε-N-allyloxycarbonyl-lysine, ε-N-propargyloxycarbonyl-lysine, ε-N-azidoethyloxycarbonyl-lysine, and ε-N-(o-azido-benzyl)-oxycarbonyl-lysine.

In some embodiments, the polypeptide described herein can be provided in form of a kit. These kits may contain an individual enzyme or a plurality of enzymes. The kits can further include reagents for carrying out the enzymatic reactions, substrates for assessing the activity of the enzymes, and reagents for detecting the products. The kits can also include instructions for the use of the kits.

In some embodiments, the polypeptides described herein can be covalently or non-covalently linked to a solid support for the purpose, for example, of screening the enzymes for activity on a range of different substrates or for facilitating the separation of reactants and products from the enzyme after the enzymatic reactions. Examples of solid supports include but are not limited to, organic polymers such as polystyrene, polyacrylamide, polyethylene, polypropylene, polyethyleneglycole, and the like, and inorganic materials such as glass, silica, controlled pore glass, metals. The configuration of the solid support can be in the form of beads, spheres, particles, gel, a membrane, or a surface.

### 5.3 Polynucleotides and Host Cells for Expression of P450 Monooxygenase Enzymes

In another aspect, the present invention provides polynucleotide molecules encoding for the improved cytochrome P450 polypeptides disclosed herein. The polynucleotides may be linked to one or more regulatory sequences controlling the expression of the cytochrome P450 polypeptide-encoding gene to form a recombinant polynucleotide capable of expressing the polypeptide.

Since the correspondence of all the possible three-base codons to the various amino acids is known, providing the amino acid sequence of the P450 polypeptide provides also a description of all the polynucleotide molecules encoding for such polypeptide. Thus, a person skilled in the art will be able, given a certain polypeptide sequence, to generate any number of different polynucleotides encoding for the same polypeptide. Preferably, the codons are selected to fit the host cell in which the polypeptide is being expressed. For example, preferred codons used in bacteria are preferably used to express the polypeptide in a bacterial host.

In some embodiments, the polynucleotide molecule comprises a nucleotide sequence encoding for a cytochrome P450 polypeptide with an amino acid sequence that is at least 60%, 70%, 80%, 85%, 90%, 95%, 99% or more identical to SEQ ID NO: 1, 2, or 3.

In some embodiments, the polynucleotide molecule encoding for the improved cytochrome P450 polypeptide is comprised in a recombinant expression vector. Examples of suitable recombinant expression vectors include but are not limited to, chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated viruses, retroviruses and many others. Any vector that transduces genetic material into a cell, and, if replication is desired, which is replicable and viable in the relevant host can be used. A large number of expression vectors and expression hosts are known in the art, and many of these are commercially available. A person skilled in the art will be able to select suitable expression vectors for a particular application, e.g., the type of expression host (e.g., in vitro systems, prokaryotic cells such as bacterial cells, and eukaryotic cells such as yeast, insect, or mammalian cells) and the expression conditions selected.

In another aspect, the present invention provides an expression host system comprising a polynucleotide molecule encoding for the improved cytochrome P450 polypeptides disclosed herein. Expression host systems that may be used within the invention include any systems that support the transcription, translation, and/or replication of a polynucleotide molecule provided herein. Preferably, the expression host system is a cell. Host cells for use in expressing the polypeptides encoded by the expression vector disclosed herein are well known in the art and include but are not limited to, bacterial cells (e.g., Escherichia coli, Streptomyces); fungal cells such as yeast cells (e.g., Saccharomyces cerevisiae, Pichia pastoris); insect cells; plant cells; and animal cells. The expression host systems also include lysates of prokaryotic cells (e.g., bacterial cells) and lysates of eukaryotic cells (e.g., yeast, insect, or mammalian cells). These systems also include in vitro transcription/translation systems, many of which are commercially available. The choice of the expression vector and host system depends on the type of application intended for the methods provided herein and a person skilled in the art will be able to select a suitable expression host based on known features and application of the different expression hosts.

### 5.4 Methods of Preparing and Using the Engineered Cytochrome P450 Polypeptides

The engineered cytochrome P450 polypeptides can be prepared via mutagenesis of the polynucleotide encoding for the naturally occurring cytochrome P450 enzymes (SEQ ID NO: 1, 2, or 3) or for an engineered variant thereof. Many mutagenesis methods are known in the art and these include, but are not limited to, site-directed mutagenesis, site-saturation mutagenesis, random mutagenesis, cassette-mutagenesis, DNA shuffling, homologous recombination, non-homologous recombination, site-directed recombination, and the like. Detailed description of art-known mutagenesis methods can be found, among other sources, in U.S. Pat. No. 5,605,793; U.S. Pat. No. 5,830,721; U.S. Pat. No. 5,834,252; WO 95/22625; WO 96/33207; WO 97/20078; WO 97/35966; WO 98/27230; WO 98/42832; WO 99/29902; WO 98/41653; WO 98/41622; WO 98/42727; WO 00/18906; WO 00/04190; WO 00/42561; WO 00/42560; WO 01/23401; WO 01/64864.

Numerous methods for making nucleic acids encoding for polypeptides having a predetermined or randomized sequence are known to those skilled in the art. For example, oligonucleotide primers having a predetermined or randomized sequence can be prepared chemically by solid phase synthesis using commercially available equipments and reagents. Polynucleotide molecules can then be synthesized and amplified using a polymerase chain reaction, digested via endonucleases, ligated together, and cloned into a vector according to standard molecular biology protocols known in the art (e.g., Sambrook et al. Molecular Cloning: A Laboratory Manual (Third Edition), Cold Spring Harbor Press, 2001). These methods, in combination with the mutagenesis methods mentioned above, can be used to generate polynucleotide molecules that encode for engineered cytochrome P450 polypeptides as well as suitable vectors for the expression of these polypeptides in a host expression system.

Engineered cytochrome P450 polypeptides expressed in a host expression system, such as, for example, in a host cell, can be isolated and purified using any one or more of the well known techniques for protein purification, including, among others, cell lysis via sonication or chemical treatment, filtration, salting-out, and chromatography (e.g., ion-exchange chromatography, gel-filtration chromatography, etc.).

The recombinant P450 polypeptides obtained from mutagenesis of a parental P450 enzyme sequences (e.g., SEQ ID NO: 1, 2, 3 or engineered variants thereof) can be screened for identifying engineered P450 polypeptides having improved enzyme properties, such as improvements with respect to their catalytic activity, coupling efficiency, regioselectivity and/or stereoselectivity for the oxidation of parthenolide. The improvement resulting from the introduced amino acid mutation(s) in any one or more of these enzyme properties can be then measured according to methods known in the art, as described above.

In some embodiments, a method is provided for oxidixing parthenolide, the method comprising
a. contacting parthenolide with an engineered cytochrome P450 polypeptide;
b. allowing for the engineered cytochrome P450 enzyme to catalyze the hydroxylation of a C—H bond within parthenolide, while preserving α-methylene-γ-lactone moiety therein, thereby producing an hydroxylated parthenolide derivative;
c. isolating the hydroxylated parthenolide derivative.

In some embodiments, the C—H bond hydroxylated by the engineered cytochrome P450 polypeptide within the method is attached to carbon C14 in parthenolide.

In some embodiments, the C—H bond hydroxylated by the engineered cytochrome P450 polypeptide within the method is attached to carbon C9 in parthenolide. In this case, and in some embodiment, either the 9(S)- or the 9(R)-hydroxy product is produced in stereomeric excess.

In some embodiments, the engineered cytochrome P450 polypeptide used in the method comprises an amino acid sequence that is at least 60%, 70%, 80%, 85%, 90%, 95%, 99% or more identical to the sequence SEQ ID NO: 1, 2, 3, 4, 5, 6, 7, 8, 10, 11, 12, 13, 14, 15, 16, 17, 18, 19, or 20.

In some embodiments, the amino acid sequence encompassing the heme domain of the engineered cytochrome P450 polypeptide used in the method comprises has an amino acid sequence that is at least 60%, 70%, 80%, 85%, 90%, 95%, 99% or more identical to the amino acid sequence encompassing the first 500 amino acids in the sequences SEQ ID NO: 1, 2, 3, 4, 5, 6, 7, 8, 10, 11, 12, 13, 14, 15, 16, 17, 18, 19, or 20 (i.e. residue 1 to residue 500 in these sequences).

As it is known in the art, P450-catalyzed reactions typically require a source of oxygen (as co-substrate) as well as a source of reducing equivalents (i.e., electrons) to drive catalysis. Most typically, and in preferred embodiments, oxygen is provided in the form of molecular oxygen. The source of reducing equivalents can be provided in the form of a soluble cofactor, and in most preferred embodiments, it is provided in the form of reduced nicotinamide adenine dinucleotide phosphate (NADPH), which is the cofactor utilized by the cytochrome P450 enzymes disclosed herein, namely the polypeptides with SEQ ID NO: 1, 2, and 3, and engineered variants thereof, as described above.

Alternative sources of reducing equivalents include but are not limited to, reduced nicotinamide adenine dinucleotide (NADH) or an electrode. Alternatively, chemical compounds that can serve as source of both oxygen and electrons such as for example, hydrogen peroxide (H₂O₂) or organic peroxides may also be used.

In some embodiments, the P450 reactions are carried out in the presence of a NADPH cofactor regeneration system or a NADH cofactor regeneration system. Suitable NADPH regeneration systems include but are not limited to, those based on glucose-6-phosphate dehydrogenase or on NADP⁺-utilizing phosphite dehydrogenase variants. (van der Donk and Zhao 2003; Zhao and van der Donk 2003) Suitable NADH regeneration systems include but are not limited to, those based on glucose dehydrogenase, phosphite dehydrogenase, or formate dehydrogenase. (van der Donk and Zhao 2003; Zhao and van der Donk 2003)

Typically, the P450 reactions are carried out in a buffered aqueous solution. Various buffering agents such as phosphate, acetate, TRIS, MOPS, HEPES, etc. can be used. An organic cosolvent such as, for example, methanol, ethanol, dimethylsulfoxide, dimethylformamide, etc. can be added, provided these cosolvent and their relative concentration in the cosolvent system does not completely inactivate the P450 enzyme.

In carrying out the P450 reactions described herein, the engineered P450 enzymes may be added to the reaction mixture in the form of purified enzymes, whole cells containing the P450 enzymes, and/or cell extracts and/or lysates of such cells.

Typically, the P450 reactions are allowed to proceed until a substantial amount of the substrate is transformed into the product. Product formation (or substrate consumption) can be monitored using standard analytical methods such as, for example, thin-layer chromatography, GC, HPLC, or LC-MS. Experimental parameters such as amount of P450 enzyme added to the reaction mixture, temperature, solvent composition, cofactor concentration, composition of the cofactor regeneration system, etc. can be readily optimized by routine experimentation and a person skilled in the art will be able to identify most suitable reaction conditions according to the substrate and the P450 enzyme utilized in the process.

### 5.5 Parthenolide Derivatives

The engineered P450 polypeptides provided herein provide a means for introducing a hydroxyl group (—OH) in aliphatic positions of the carbocyclic backbone of parthenolide, such as position C9 or position C14, whose chemical functionalization have never been accomplished before. According to the methods provided herein, the enzymatically installed hydroxyl group can be converted into a variety of other functional groups through versatile methods for chemical hydroxyl group interconversion, such as nucleophilic substitution (e.g., Mitsunobu substitution), alkylation, acylation, deoxyhalogenation, O—H carbene insertion, and the like.

Accordingly, parthenolide derivatives are provided that are modified at the level of carbon atom C9 or C14. Furthermore, parthenolide derivatives are provided that are (doubly) functionalized at the level of carbon atoms C9 and C13 or at the level of carbon atoms C14 and C13. Notably, some of these compounds were found to possess significantly improved anticancer activity compared to PTL, while others combined improved anticancer activity with increased water solubility as compared to PTL.

A compound of general formula (I) or salt thereof is provided:

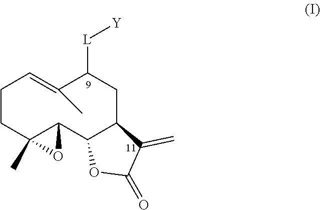

where
L represents —O—, —NH—, —NHC(O)—, —OC(O)—, —OC(O)NH—, —S—, —SO—, —SO₂—, —PO—, —OCH₂—, or simply a chemical bond connecting the carbon atom to Y; and Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or
Y is absent and L represents a halogen atom, an azido group (—N₃), an optionally substituted triazole group, or L represents a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R³ and R⁴ are connected together to form an optionally substituted heterocyclic group.

A compound of general formula (II) or salt thereof is also provided:

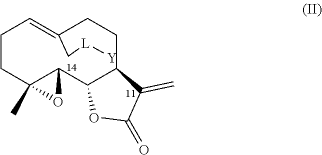

where
L represents —O—, —NH—, —NHC(O)—, —OC(O)—, —OC(O)NH—, —S—, —SO—, —SO₂—, —PO—, —OCH₂—, or simply a chemical bond connecting the carbon atom to Y; and Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or
Y is absent and L represents a halogen atom, an azido group (—N₃), an optionally substituted triazole group, or L represents a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R³ and R⁴ are connected together to form an optionally substituted heterocyclic group.

A compound of general formula (III) or salt thereof is also provided:

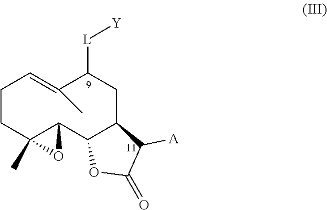

in which
A is —CH₂R* wherein R* is an amino acid residue bonded to the A methylene via a nitrogen or sulfur atom; or R* is —NR¹R², —NR¹C(O)R², —NR¹CO₂R², or —SR¹, wherein
R¹ and R² are independently selected from the group consisting of H and an optionally substituted alkyl, alkenyl, or alkynyl group, an optionally substituted heteroalkyl, heteroalkenyl, or heteroalkynyl group, an optionally substituted aryl group, an optionally substituted heteroaryl group, and an optionally substituted heterocyclic group; or where R* is —NR¹R², R₁ and R₂ optionally together with the nitrogen atom form a an optionally substituted 5-12 membered ring, the ring optionally comprising one or more heteroatoms or a group selected from the group consisting of —CO—, —SO—, —SO₂—, and —PO—; and
L represents —O—, —NH—, —NHC(O)—, —OC(O)—, —OC(O)NH—, —S—, —SO—, —SO₂—, —PO—, —OCH₂—, or simply a chemical bond connecting the carbon atom to Y; and Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or
Y is absent and L represents a halogen atom, an azido group (—N₃), an optionally substituted triazole group, or L represents a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R³ and R⁴ are connected together to form an optionally substituted heterocyclic group.

A compound of general formula (IV) or salt thereof is also provided:

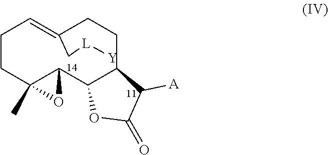

in which
A is —CH₂R* wherein R* is an amino acid residue bonded to the A methylene via a nitrogen or sulfur atom; or R* is —NR¹R², —NR¹C(O)R², —NR¹CO₂R², or —SR¹, wherein
R¹ and R² are independently selected from the group consisting of H and an optionally substituted alkyl, alkenyl, or alkynyl group, an optionally substituted heteroalkyl, heteroalkenyl, or heteroalkynyl group, an optionally substituted aryl group, an optionally substituted heteroaryl group, and an optionally substituted heterocyclic group; or where R* is —NR¹R², R₁ and R₂ optionally together with the nitrogen atom form a an optionally substituted 5-12 membered ring, the ring optionally comprising one or more heteroatoms or a group selected from the group consisting of —CO—, —SO—, —SO₂—, and —PO—; and
L represents —O—, —NH—, —NHC(O)—, —OC(O)—, —OC(O)NH—, —S—, —SO—, —SO₂—, —PO—, —OCH₂—, or simply a chemical bond connecting the carbon atom to Y; and Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or
Y is absent and L represents a halogen atom, an azido group (—N₃), an optionally substituted triazole group, or L represents a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R³ and R⁴ are connected together to form an optionally substituted heterocyclic group.

Salts of the compounds provided herein can be prepared according to standard procedures well known in the art, for example, by reacting a compound containing a one or more sufficiently basic functional group with a suitable organic or mineral acid. Similarly, base addition salts can be prepared by reacting a compound containing a one or more sufficiently acid functional group with a suitable organic or mineral base. Examples of inorganic acid addition salts includes fluoride, chloride, bromide, iodide, sulfate, nitrate, bicarbonate, phosphate, and carbonate salts. Examples of organic acid addition salts include acetate, citrate, malonate, tartrate, succinate, lactate, malate, benzoate, ascorbate, α-ketoglutarate, tosylate, and methanesulfonate salts. Examples of base addition salts include lithium, sodium, potassium, calcium, and ammonium salts.

In specific embodiments, the substituent L in the compounds of general formula I and II is —OC(O)— and the substituent Y is phenyl, 4-pyridyl, (4-dimethylamino)phenyl, para-, meta-, or ortho-fluoro-phenyl, para-, meta-, or ortho-trifluoromethyl-phenyl, (2,4-bis-trifluoromethyl)phenyl, (3,5-bis-trifluoromethyl)phenyl, 1- or 2-naphyl, 3-N-methyl-indolyl, 5-(4-chlorophenyl)isoxazolyl, 2-(4-bromophenyl)furanyl, 2-(2-(trifluoromethyl)phenyl)furanyl, or thiophene group.

In other specific embodiments, the substituent L in the compounds of general formula I and II is —O— and the substituent Y is (phenyl)methyl, (4-pyridyl)methyl, (4-dimethylaminophenyl)methyl, (para-, meta-, or ortho-fluoro-phenyl)methyl, (para-, meta-, or ortho-trifluoromethyl-phenyl)methyl, (2,4-bis-trifluoromethyl-phenyl)methyl, (3,5-bis-trifluoromethyl-phenyl)methyl, (naphyl)methyl, (3-N-methyl-indolyl)methyl, (5-(4-chlorophenyl)isoxazolyl)methyl, (2-(4-bromophenyl)furanyl)methyl, (2-(2-(trifluoromethyl)phenyl)furanyl)methyl, or methyl(thiophene) group.

In other specific embodiments, the substituent L in the compounds of general formula I and II is —O— and the substituent Y is a group —CH(Ar′)COOR′, wherein Ar′ is selected from the group consisting of phenyl, 4-pyridyl, (4-dimethylamino)phenyl, para-, meta-, or ortho-fluoro-phenyl, para-, meta-, or ortho-trifluoromethyl-phenyl, (2,4-bis-trifluoromethyl)phenyl, (3,5-bis-trifluoromethyl)phenyl, 1- or 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene group; and the R′ group is selected from the group consisting of methyl, ethyl, propyl, isopropyl, tert-butyl, benzyl, 2-morpholinoethyl, 2-morpholinoethyl, 2-(piperidin-1-yl)ethyl, and 2-(pyrrolidin-1-yl)ethyl group.

In other specific embodiments, the substituent L in the compounds of general formula III and IV is —OC(O)—; the substituent Y is selected from the group consisting of phenyl, 4-pyridyl, (4-dimethylamino)phenyl, para-, meta-, or ortho-fluoro-phenyl, para-, meta-, or ortho-trifluoromethyl-phenyl, (2,4-bis-trifluoromethyl)phenyl, (3,5-bis-trifluoromethyl)phenyl, 1- or 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene group;

and the substituent A is —CH₂R*, where R* is selected from the group consisting of methylamino (—NH(CH₃)), dimethylamino (—N(CH₃)₂), methylethylamino (—N(CH₃)(CH₂CH₃)), methylpropylamino (—N(CH₃)(CH₂CH₂CH₃)), methylisopropylamino (—N(CH₃)(CH₂(CH₃)₂), —N(CH₃)(CH₂CH₂OH), pyrrolidine, piperidine, 4-methylpiperidine, 1-phenylmethanamine (—NCH₂Ph), and 2-phenylethanamine (—NCH₂CH₂Ph).

In other specific embodiments, the substituent L in the compounds of general formula III and IV is —O—; the substituent Y is selected from the group consisting of (phenyl)methyl, (4-pyridyl)methyl, (4-dimethylaminophenyl)methyl, (para-, meta-, or ortho-fluoro-phenyl)methyl, (para-, meta-, or ortho-trifluoromethyl-phenyl)methyl, (2,4-bis-trifluoromethyl-phenyl)methyl, (3,5-bis-trifluoromethyl-phenyl)methyl, (naphyl)methyl, (3-N-methyl-indolyl)methyl, (5-(4-chlorophenyl)isoxazolyl)methyl, (2-(4-bromophenyl)furanyl)methyl, (2-(2-(trifluoromethyl)phenyl)furanyl)methyl, methyl(thiophenene) group, and a —CH(Ar′)COOR′ group, wherein Ar′ is selected from the group consisting of phenyl, 4-pyridyl, (4-dimethylamino)phenyl, para-, meta-, or ortho-fluoro-phenyl, para-, meta-, or ortho-trifluoromethyl-phenyl, (2,4-bis-trifluoromethyl)phenyl, (3,5-bis-trifluoromethyl)phenyl, 1- or 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene group; and the R′ group is selected from the group consisting of methyl, ethyl, propyl, isopropyl, tert-butyl, benzyl, 2-morpholinoethyl, 2-morpholinoethyl, 2-(piperidin-1-yl)ethyl, and 2-(pyrrolidin-1-yl)ethyl group; and the substituent A is —CH₂R*, where R* is selected from the group consisting of methylamino (—NH(CH₃)), dimethylamino (—N(CH₃)₂), methylethylamino (—N(CH₃)(CH₂CH₃)), methylpropylamino (—N(CH₃)(CH₂CH₂CH₃)), methylisopropylamino (—N(CH₃)(CH₂(CH₃)₂), —N(CH₃)(CH₂CH₂OH), pyrrolidine, piperidine, 4-methylpiperidine, 1-phenylmethanamine (—NCH₂Ph), and 2-phenylethanamine (—NCH₂CH₂Ph).

A person skilled in the art will promptly recognize that several different chemical methods, including different chemical reagents and reaction conditions, are available for synthesizing the compounds of general formula I and II, once the hydroxylated parthenolide derivatives, i.e. 9-hydroxy-parthenolide (3) and 14-hydroxyparthenolide (4), respectively, are made available. Accordingly, this invention focuses on the products of these transformations rather than on the specific chemical methods applied to achieve them, which of course can vary. It should be noted, however, that the examples included in this disclosure demonstrate the feasibility of applying common, art-known chemical transformations for hydroxyl group functional interconversion for the preparation of compounds of general formula I and II. These include substitution or functionalization of the hydroxyl group in compound 3 and 4 via alkylation, methylation, acylation, nucleophilic substitution (e.g., Mitsunobu substitution), and deoxohalogenation (e.g., deoxofluorination). As described below, compounds of general formula III and IV can be obtained by further modifying the derivatives of general formula I and II, respectively, at the C13 position according to procedures known in the art.

Compounds of general formula I and II are prepared by first subjecting parthenolide to a reaction with a suitable P450 polypeptide in order to produce 9-hydroxy-parthenolide (compound 3) or 14-hydroxy-parthenolide (compound 4). Typically, these reactions are carried out in aqueous buffer at near-neutral pH (typically, phosphate buffer, pH 8.0) with varying amount (typically, up to 20%) of an organic solvent (typically, DMSO) to facilitate dissolution of parthenolide in the buffer. Either NAPDH or, most preferably, a NADPH cofactor regeneration system is included to provide the reducing equivalents to support the P450 reaction. Typically, a NADPH cofactor regeneration system is used, which consists of phosphite dehydrogenase, NADP⁺, and sodium phosphite. The reaction temperature can be from 4 to 50 degree Celsius. The reaction time and concentration of the P450 polypeptide in the reaction mixture can vary widely, in large part depending on the stability, catalytic rate and, catalytic efficiency of the P450 enzyme. Typically, reaction times range from 1 to 48 hours, whereas the P450 catalyst concentration range from 0.1 to 10 mol %. Purification of the hydroxylation products can be achieved by a variety of techniques, such as by normal phase liquid chromatography through silica gel; reverse-phase liquid chromatography through bonded silica gel such as octadecylsilica, octylsilica and the like; and recrystallization using pure organic solvents or solvent mixtures. After isolation, the hydroxylated parthenolide derivatives provided herein (i.e. compounds 3 and 4) can be subjected to suitable chemical reagents and reaction conditions to functionalize or substitute the hydroxyl group in C9 or C14 with a different substituent. As mentioned above, a person skilled in the art will be able to readily select such reagents and reaction conditions for the purpose of preparing compounds of general formula I and II from 9- and 14-hydroxy-parthenolide, respectively. For example, 9- and 14-ester derivatives can be prepared via acylation of 9-hydroxy-parthenolide and 14-hydroxy-parthenolide, respectively, with an acid chloride in dichloromethane in the presence of a weakly nucleophilic base (e.g., triethylamine, triisopropylamine, or pyridine). Alternatively, such ester derivatives can be prepared via reaction with a free acid in dichloromethane in presence of a coupling reagent (e.g., dicyclohexylcarbodiimide or DCC, O-(Benzotriazol-1-yl)-N,N,N′,N′-tetramethyluronium hexafluorophosphate or HBTU), and weakly nucleophilic base (e.g., triethylamine, triisopropylamine, or pyridine). Optionally, coupling catalysts (e.g., 4-dimethylamino-pyridine or DMAP, 1-Hydroxybenzotriazole or HOBt) can be added to facilitate the esterification reaction.

Doubly substituted parthenolide derivatives of general formula III and IV can be prepared by further modifying the compounds of general formula I and II described above at the reactive position C13 according to procedures known in the art. Procedures that are useful for modification of position C13 in these compounds can be found, among other sources, in the following references. (Guzman, Rossi et al. 2006; Hwang, Chang et al. 2006; Nasim and Crooks 2008; Han, Barrios et al. 2009; Neelakantan, Nasim et al. 2009; Woods, Mo et al. 2011) Additional procedures suitable for C13 modification in parthenolide are described in Crooks et. al, U.S. Pat. No. 7,312,242; U.S. Pat. No. 7,678,904; U.S. Pat. No. 8,124,652. Preferably, for the purpose of preparing compounds of general formula III and IV, these aforementioned procedures for C13 modification are chosen so that they do not alter or react with any of the functional groups comprised by the substituents installed in position C9 or C14 of the parthenolide derivatives of formula I or II, respectively. A person skilled in the art will be able to choose or adapt, if necessary, suitable procedures for this purpose.

### 5.6 Use of Parthenolide Derivatives for Treatment of Cancer and Other Diseases

The invention also provides a pharmaceutical composition comprising an effective amount of a compound of formula I, II, III, or IV, or a pharmaceutically acceptable salt, ester or prodrug thereof, in combination with a pharmaceutically acceptable diluent or carrier.

The invention also provides a method of inhibiting cancer cell growth and metastasis of cancer cells, comprising administering to a mammal afflicted with cancer, an amount of a compound of formula I, II, III, or IV, effective to inhibit the growth of the cancer cells.

The invention also provides a method comprising inhibiting cancer cell growth by contacting the cancer cell in vitro or in vivo with an amount of a compound of formula I, II, III, or IV, effective to inhibit the growth of the cancer cell.

The invention also provides a compound of formula (I) for use in medical therapy (preferably for use in treating cancer, e.g., solid tumors), as well as the use of such compound for the manufacture of a medicament useful for the treatment of cancer and other diseases/disorders described herein.

The invention further provides methods of treating inflammatory diseases and disorders, including, for example, rheumatoid arthritis, osteoarthritis, allergies (such as asthma), and other inflammatory conditions, such as pain (such as migraine), swelling, fever, psoriasis, inflammatory bowel disease, gastrointestinal ulcers, cardiovascular conditions, including ischemic heart disease and atherosclerosis, partial brain damage caused by stroke, skin conditions (eczema, sunburn, acne), leukotriene-mediated inflammatory diseases of lungs, kidneys, gastrointestinal tract, skin, prostatitis and paradontosis.

The invention further provides methods of treating immune response disorders, whereby the immune response is inappropriate, excessive or lacking. Such disorders include allergic responses, transplant rejection, blood transfusion reaction, and autoimmune disorders including, but not limited to, Addison's disease, alopecia areata, antiphospholipid antibody syndrome (aPL), autoimmune hepatitis, celiac disease-sprue (gluten-sensitive enteropathy), dermatomyositis, Graves' disease, Guillain-Barre syndrome, Hashimoto's disease, hemolytic anemia, idiopathic thrombocytopenic purpura, inflammatory bowel disease (IBD), inflammatory myopathies, multiple sclerosis, myasthenia gravis, pernicious anemia, primary biliary cirrhosis, psoriasis, reactive arthritis, rheumatoid arthritis, scleroderma, Sjögren's syndrome, systemic lupus erythematosus, Type I diabetes and vitiligo.

The compounds disclosed herein are useful for treating cancer. Cancers treatable by the present therapy include the solid and hematological tumors, such as leukemia, breast cancer, lung cancer, prostate cancer, colon cancer, bladder cancer, liver cancer, skin cancer, brain cancer, pancreas cancer, kidney cancer, and bone cancer, comprising administering to a mammal afflicted with the cancer an amount of parthenolide derivative effective to inhibit the viability of cancer cells of the mammal. The parthenolide derivative may be administered as primary therapy, or as adjunct therapy, either following local intervention (surgery, radiation, local chemotherapy) or in conjunction with another chemotherapeutic agent. Hematological cancers, such as the leukemias are disclosed in the Mayo Clinic Family Health Book, D. E. Larson, ed., William Morrow, N.Y. (1990) and include CLL, ALL, CML and the like. Compounds of the present invention may be used in bone marrow transplant procedure to treat bone marrow prior to reintroduction to the patient. In addition, the compounds of the present invention may be used as chemotherapy sensitizers or radiation therapy sensitizers. Accordingly, a patient, or cells, or tissues, derived from a cancer patient, are pre-treated with the compounds prior to standard chemotherapy or radiation therapy.

Given the demonstrated activity of DMAPT for treating cancer, the compounds disclosed herein can be useful for treating cancer.

Within another aspect of the present invention, methods are provided for inhibiting angiogenesis in patients with non-tumorigenic, angiogenesis-dependent diseases, comprising administering a therapeutically effective amount of a composition comprising parthenolide derivative to a patient with a non-tumorigenic angiogenesis-dependent disease, such that the formation of new blood vessels is inhibited. Within other aspects, methods are provided for inhibit reactive proliferation of endothelial cells or capillary formation in non-tumorigenic, angiogenesis-dependent diseases, such that the blood vessel is effectively occluded. Within one embodiment, the anti-angiogenic composition comprising parthenolide derivative is delivered to a blood vessel which is actively proliferating and nourishing a tumor.

In addition to tumors, numerous other non-tumorigenic angiogenesis-dependent diseases, which are characterized by the abnormal growth of blood vessels, may also be treated with the anti-angiogenic parthenolide derivative compositions, or anti-angiogenic factors of the present invention. Anti-angiogenic parthenolide derivative compositions of the present invention can block the stimulatory effects of angiogenesis promoters, reducing endothelial cell division, decreasing endothelial cell migration, and impairing the activity of the proteolytic enzymes secreted by the endothelium. Representative examples of such non-tumorigenic angiogenesis-dependent diseases include corneal neovascularization, hypertrophic scars and keloids, proliferative diabetic retinopathy, arteriovenous malformations, atherosclerotic plaques, delayed wound healing, hemophilic joints, nonunion fractures, Osler-Weber syndrome, psoriasis, pyogenic granuloma, scleroderma, trachoma, menorrhagia, retrolental fibroplasia and vascular adhesions. The pathology and treatment of these conditions is disclosed in detail in published PCT application PCT/CA94/00373 (WO 95/03036). Topical or directed local administration of the present compositions is often the preferred mode of administration of therapeutically effective amounts of parthenolide derivative, i.e., in depot or other controlled release forms.

Anti-angiogenic compositions of the present invention may also be utilized in a variety of other manners. For example, they may be incorporated into surgical sutures in order to prevent stitch granulomas, implanted in the uterus (in the same manner as an IUD) for the treatment of menorrhagia or as a form of female birth control, administered as either a peritoneal lavage fluid or for peritoneal implantation in the treatment of endometriosis, attached to a monoclonal antibody directed against activated endothelial cells as a form of systemic chemotherapy, or utilized in diagnostic imaging when attached to a radioactively labelled monoclonal antibody which recognizes active endothelial cells. The magnitude of a prophylactic or therapeutic dose of parthenolide derivative, an analog thereof or a combination thereof, in the acute or chronic management of cancer, i.e., prostate or breast cancer, will vary with the stage of the cancer, such as the solid tumor to be treated, the chemotherapeutic agent(s) or other anti-cancer therapy used, and the route of administration. The dose, and perhaps the dose frequency, will also vary according to the age, body weight, and response of the individual patient. In general, the total daily dose range for parthenolide derivative and its analogs, for the conditions described herein, is from about 0.5 mg to about 2500 mg, in single or divided doses. Preferably, a daily dose range should be about 1 mg to about 100 mg, in single or divided doses, most preferably about 5-50 mg per day. In managing the patient, the therapy should be initiated at a lower dose and increased depending on the patient's global response. It is further recommended that infants, children, patients over 65 years, and those with impaired renal or hepatic function initially receive lower doses, and that they be titrated based on global response and blood level. It may be necessary to use dosages outside these ranges in some cases. Further, it is noted that the clinician or treating physician will know how and when to interrupt, adjust or terminate therapy in conjunction with individual patient response. The terms “an effective amount” or “an effective sensitizing amount” are encompassed by the above-described dosage amounts and dose frequency schedule.

Any suitable route of administration may be employed for providing the patient with an effective dosage of parthenolide derivative (e.g., oral, sublingual, rectal, intravenous, epidural, intrathecal, subcutaneous, transcutaneous, intramuscular, intraperitoneal, intracutaneous, inhalation, transdermal, nasal spray, nasal gel or drop, and the like). While it is possible that, for use in therapy, parthenolide derivative or its analogs may be administered as the pure chemicals, as by inhalation of a fine powder via an insufflator, it is preferable to present the active ingredient as a pharmaceutical formulation. The invention thus further provides a pharmaceutical formulation comprising parthenolide derivative or an analog thereof, together with one or more pharmaceutically acceptable carriers therefor and, optionally, other therapeutic and/or prophylactic ingredients. The carrier(s) must be ‘acceptable’ in the sense of being compatible with the other ingredients of the formulation and not deleterious to the recipient thereof, such as a human patient or domestic animal.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

Pharmaceutical formulations suitable for oral administration may be presented as discrete unit dosage forms such as hard or soft gelatin capsules, cachets or tablets each containing a predetermined amount of the active ingredient; as a powder or as granules; as a solution, a suspension or as an emulsion; or in a chewable base such as a synthetic resin or chicle for ingestion of the agent from a chewing gum. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art, i.e., with enteric coatings.

Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g., by injection, for example, bolus injection or continuous infusion) and may be presented in unit dose form in ampules, pre-filled syringes, small volume infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds may be formulated as ointments, creams or lotions, or as the active ingredient of a transdermal patch. Suitable transdermal delivery systems are disclosed, for example, in Fisher et al. U.S. Pat. No. 4,788,603, or Bawa et al. U.S. Pat. Nos. 4,931,279; 4,668,506 and 4,713,224. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described formulations can be adapted to give sustained release of the active ingredient employed, e.g., by combination with certain hydrophilic polymer matrices, e.g., comprising natural gels, synthetic polymer gels or mixtures thereof. The polymer matrix can be coated onto, or used to form, a medical prosthesis, such as a stent, valve, shunt, graft, or the like.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in molds.

Formulations suitable for vaginal administration may be presented as pessaries, tampons, creams, gels, pastes, foams or sprays containing, in addition to the active ingredient, such carriers as are known in the art to be appropriate.

For administration by inhalation, the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds according to the invention may take the form of a dry powder composition, for example, a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the compounds provided herein may be administered via a liquid spray, such as via a plastic bottle atomizer. Typical of these are the Mistometer® (Wintrop) and the Medihaler® (Riker).

For topical administration to the eye, the compounds can be administered as drops, gels (U.S. Pat. No. 4,255,415), gums (see U.S. Pat. No. 4,136,177) or via a prolonged-release ocular insert.

The term “treatment” refers to any treatment of a pathologic condition in a mammal, particularly a human, and includes: (i) preventing the pathologic condition from occurring in a subject which may be predisposed to the condition but has not yet been diagnosed with the condition and, accordingly, the treatment constitutes prophylactic treatment for the disease condition; (ii) inhibiting the pathologic condition, i.e., arresting its development; (iii) relieving the pathologic condition, i.e., causing regression of the pathologic condition; or (iv) relieving the conditions mediated by the pathologic condition.

The term “therapeutically effective amount” refers to that amount of a compound of the invention that is sufficient to effect treatment, as defined above, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “pharmaceutically acceptable salts” includes, but is not limited to, salts well known to those skilled in the art, for example, mono-salts (e.g., alkali metal and ammonium salts) and poly salts (e.g., di- or tri-salts,) of the compounds of the invention. Pharmaceutically acceptable salts of compounds of formulas I, II, III, or IV are where, for example, an exchangeable group, such as hydrogen in —OH, —NH—, or —P(═O)(OH)—, is replaced with a pharmaceutically acceptable cation (e.g., a sodium, potassium, or ammonium ion) and can be conveniently be prepared from a corresponding compound of formula I, II, III, or IV by, for example, reaction with a suitable base. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

The compounds provided herein may contain one or more chiral centers. Accordingly, the compounds are intended to include racemic mixtures, diastereomers, enantiomers, and mixture enriched in one or more stereoisomer. When a group of substituents is disclosed herein, all the individual members of that group and all subgroups, including any isomers, enantiomers, and diastereomers are intended to be included in this disclosure. Additionally, all isotopic forms of the compounds disclosed herein are intended to be included in this disclosure. For example, it is understood that any one or more hydrogens in a molecule disclosed herein can be replaced with deuterium or tritium.

A person skilled in the art will also appreciate that starting materials, biological materials, reagents, synthetic methods, purification methods, analytical methods, assay methods, and biological methods other than those specifically exemplified can be employed in the practice of the invention. All art-known functional equivalents of any such materials and methods are intended to be included in the invention.

Unless defined otherwise herein, all technical and scientific terms used herein have the same meaning as commonly understood by one of ordinary skill in the art to which this disclosure pertains.

The following examples are offered by way of illustration and not by way of limitation.

### 6. EXAMPLES

### 6.1 Example 1

### Isolation of Engineered P450 Polypeptides for Parthenolide Hydroxylation

In initial studies, we discovered that CYP102A1 variant FL#62 (SEQ ID NO: 12) is capable of efficiently oxidizing PTL, supporting more than 1,000 total turnovers (TTN) and producing a mixture of 1,10-epoxy-PTL (compound 2), 9(S)-hydroxy-PTL (compound 3), 14-hydroxy-PTL (compound 4) in 77:13:10 ratio (FIG. 1 and Table 1). The hydroxylation products 3 and 4 were of particular interest, as they can provide two valuable intermediates, not accessible via currently available synthetic methods, for re-elaboration of parthenolide carbocyclic skeleton by chemoenzymatic means. A collection of about 500 FL#62-derived P450s were obtained via a two step process involving (a) simultaneous site-saturation mutagenesis of multiple ‘first-sphere’ active-site residues (i.e. 74, 78, 81, 82, 87, 181, and 184), followed by (b) high-throughput mapping of the active site configuration of the resulting engineered P450 variants by means of a panel of five structurally diverse chromogenic probes (Zhang, El Damaty et al. 2011; Zhang, Shafer et al. 2012). Additional engineered P450 libraries based on CYP102A5 (SEQ ID NO: 2) and CYP505X (SEQ ID NO: 3) were prepared in a similar manner by mutagenesis of one or more of the amino acid positions listed in Section 5.2 of this application. Selected P450 variants from these libraries were tested for improved activity and selectivity toward parthenolide hydroxylation at position C9 and/or C14. Typically, parthenolide hydroxylation activity was determined by reactions with the P450 variant (1 μM in purified form or in cell lysate) in buffered solution (50 mM potassium phosphate, pH 8.0) in the presence of 1 mM parthenolide and a NADPH cofactor regeneration system (2 μM phosphite dehydrogenase, 150 μM NADP⁺, 50 mM sodium phosphite). The enzymatic reactions were extracted with dichloromethane and analyzed by gas chromatography. As described in Table 1, several P450 variants derived from CYP102A1 (SEQ ID NO: 1), CYP102A5 (SEQ ID NO: 2) or CYP505X (SEQ ID NO: 3) were found to exhibit improved activity and/or selectivity in the hydroxylation of parthenolide.

TABLE 1

| | Product | | | | | |
|---|---|---|---|---|---|---|
| | Distribution (%) | | | | | Amino acid mutations (vs parent |
| P450 | 2 | 3 | 4 | Turnovers | Turnoversmin⁻¹ | enzyme)^{a} |
| CYP102A1 | 100% | 0% | 0% | 29 | <5 | — |
| FL#41 | 77% | 23% | 0% | 76 | n.d. | vs CYP102A1: F87A |
| FL#44 | 61% | 2% | 37% | 493 | n.d. | vs CYP102A1: V78A, H138Y, |
| | | | | | | T175I, V178I, A184V, H236Q, |
| | | | | | | E252G, R255S, A290V, A295T, |
| | | | | | | L353V |
| FL#45 | 72% | 6% | 22% | 484 | n.d. | vs CYP102A1: V78A, T175I, |
| | | | | | | A184V, F205C, S226R, H236Q, |
| | | | | | | E252G, R255S, A290V, L353V |
| FL#46 | 41% | 0% | 59% | 420 | n.d. | vs CYP102A1: R47C, V78A, F87I, |
| | | | | | | K94I, P142S, T175I, A184V, F205C, |
| | | | | | | S226R, H236Q, E252G, R255S, |
| | | | | | | A290V, L353V |
| FL#47 | 91% | 4% | 5% | 500 | n.d. | vs CYP102A1: R47C, V78A, F87A, |
| | | | | | | K94I, P142S, T175I, A184V, F205C, |
| | | | | | | S226R, H236Q, E252G, R255S, |
| | | | | | | A290V, L353V |
| FL#48 | 67% | 24% | 9% | 58 | n.d. | vs CYP102A1: R47C, V78A, A82L, |
| | | | | | | K94I, P142S, T175I, A184V, F205C, |
| | | | | | | S226R, H236Q, E252G, R255S, |
| | | | | | | A290V, L353V |
| FL#55 | 95% | 0% | 5% | 45 | n.d. | vs CYP102A1: R47C, L52I, A74P, |
| | | | | | | V78F, A82S, K94I, P142S, T175I, |
| | | | | | | A184S, L188P, F205C, S226R, |
| | | | | | | H236Q, E252G, R255S, A290V, |
| | | | | | | A328F, L353V, I366V, E464G, |
| | | | | | | I710T |
| FL#59 | 88% | 7% | 5% | 496 | n.d. | vs CYP102A1: V78A, F81W, A82S, |
| | | | | | | F87A, P142S, T175I, A184V, A197V, |
| | | | | | | F205C, S226R, H236Q, E252G, |
| | | | | | | R255S, A290V, L353V |
| FL#62 | 77% | 13% | 10% | 1042 | 234 | vs CYP102A1: V78A, A82V, F81R, |
| | | | | | | F87A, P142S, T175I, A180T, |
| | | | | | | A184V, A197V, F205C, S226R, |
| | | | | | | H236Q, E252G, R255S, A290V, |
| | | | | | | L353V |
| II-C5 | 29% | 68% | 3% | 1370 | 59 | vs FL#62: A78T, S81I, V82A |
| II-E2 | 26% | 20% | 53% | 1055 | 176 | vs FL#62: A87N, S81F, V82A |
| VII-H11 | 17% | 2% | 81% | 420 | 21 | vs FL#62: A87N, S81F, V82A, |
| | | | | | | T180A, L181A, V184S |
| 5A1 | 32% | 64% | 4% | 369 | n.d. | vs FL#62: A78T, S81I, V82A, T180A |
| XI-A11 | 22% | 77% | 1% | 1710 | 44 | vs FL#62: A78T, S81I, V82T |
| XII-D8 | 4% | 0% | 95% | 60 | 2 | vs FL#62: A87N, S81F, V82A, |
| | | | | | | A87V, T180A, L181A, V184S |
| XII-F12 | 19% | 80% | 1% | 1310 | 27 | vs IIC5: A82T, 180A |
| IIC5(82T/ | 22% | 78% | 0% | 59 | n.d. | vs IIC5: A82T, A87S, T180A |
| 87S/180A) | | | | | | |
| CYP102 | 100% | 0% | 0% | 206 | n.d. | vs CYP102A5: F90A, L184A, A187L |
| A5(F90A/ | | | | | | |
| L184A/ | | | | | | |
| A187L) | | | | | | |
| CYP102 | 33% | 0% | 67% | 5 | n.d. | vs CYP102A5: V81A, A85P |
| A5(V81A/ | | | | | | |
| A85P) | | | | | | |
| CYP505 | 85% | 10% | 5% | 50 | n.d. | vs CYP505X: F91A |
| X(F91A) | | | | | | |

^{a}The amino acid numbering scheme for the CYP102A1 variants corresponds to that commonly used in the literature, in which the first amino acid after the initial Methionine (i.e., Thr2) is referred to as Thr1.

Experimental Details.

The P450 enzymes were expressed from pCWori-based vectors and purified by ion-exchange chromatography according to established procedures (Zhang, El Damaty et al. 2011; Zhang, Shafer et al. 2012). P450 concentration was determined from CO binding difference spectra (ε₄₅₀₋₅₀₀=91,000 M⁻¹ cm⁻¹). Site-saturation libraries were prepared using mutagenizing primers (NNK codon at target position) according to standard cloning procedures as described for example in (Zhang, El Damaty et al. 2011; Zhang, Shafer et al. 2012). To determine total turnover numbers and regioselectivity of the P450 variants, analytical-scale reactions (1 mL) were carried out using 0.2-1 μM P450, 1.5 mM parthenolide, 2 μM PTDH, 100 μM NADP⁺, and 50 mM sodium phosphite in potassium phosphate buffer (50 mM, pH 8.0). The P450 variants were characterized either in purified form or directly from cell lysates. After 12 hours at 4° C., the reaction mixtures were added with 500 μM guaiacol (as internal standard), extracted with dichloromethane and analyzed by gas chromatography (GC). GC analyses were carried out on a Shimadzu GC2010, an FID detector, a Restek RTX-5 column (15 m×0.25 mm×0.25 μm film), and the following separation program: 200° C. inlet, 300° C. detector, 130° C. oven, 12° C./min ramp to 290° C., and 290° C. for 2 min. TTN values were calculated based on the total amount of oxidation products as quantified based on the calibration curves generated using purified 2-4. Initial product formation rates were measured from 1 mL scale reactions containing 1 mM parthenolide, 0.1-1.0 μM purified P450, and 1 mM NADPH in potassium phosphate buffer (50 mM, pH 8.0) at room temperature. After 60 seconds, the samples were added with 500 μM Guaiacol and extracted with dichloromethane. Cofactor oxidation rate in the presence of parthenolide was measured by monitoring NADPH depletion at 340 nm (c=6.22 mM⁻¹ cm⁻¹) using 0.1-0.5 μM purified P450, 1.0 mM parthenolide, and 200 μM NADPH. Coupling efficiency was calculated from the ratio between the initial product formation rate and the initial NADPH oxidation rate.

### 6.2 Example 2

### Synthesis of 9-hydroxy-parthenolide and 14-hydroxy-parthenolide Using Purified Enzyme

This example demonstrates how engineered P450 polypeptides provided herein are useful for enabling the synthesis of the derivative 9-hydroxy-parthenolide and 14-hydroxy-parthenolide at preparative scales.

General Conditions for Enzymatic Reactions:

To phosphate buffer (50 mM, pH 8.0) was added P450 (2 μM), parthenolide (1 mM), NADP⁺ (150 μM), PTDH (2 μM), and sodium phosphite (50 mM, pH 8.0). After stirring for 12 hours at room temperature, the reactions were extracted with dichloromethane (3×30 mL) and separated via centrifugation. The combined organic layers were dried over sodium sulfate, concentrated under reduced pressure, and purified by silica gel flash chromatography (10 to 60% ethyl acetate in hexanes).

To prepare 9(S)-hydroxy-parthenolide (3), purified P450 variant XII-F12 (SEQ ID NO: 19) (final conc: 2.5 μM; 0.26 mol %) was dissolved in 400 mL 50 mM phosphate buffer (pH 8.0) in the presence of parthenolide (100 mg, final conc.: 0.95 mM), PTDH (2 μM), NADP⁺ (150 μM), and sodium phosphite (50 mM). The reaction mixture was stirred for 12 hours at 4° C. The crude product was extracted with dichloromethane (3×80 mL). The collected organic layers were dried with sodium sulfate, concentrated under vacuum, and purified by flash chromatography (hexanes/ethyl acetate: 1/2) to afford 3 (75 mg, 70%) and 2 (16 mg, 15%). 9(S)-hydroxy-parthenolide (3): ¹H NMR (500 MHz, CDCl₃): δ 1.34 (s, 3H), 1.76 (s, 3H), 1.97-2.06 (m, 1H), 2.15-2.27 (m, 4H), 2.50 (dq, 1H, J=5.2, 12.4 Hz), 2.70 (d, 1H, J=8.7 Hz), 2.83-2.90 (m, 1H), 3.86 (t, 1H, J=8.5 Hz), 4.27 (dd, 1H, J=2.2, 10.5 Hz), 5.42 (d, 1H, J=11.3 Hz), 5.69 (d, 1H, J=3.2 Hz), 6.36 (d, 1H, J=3.6 Hz); ¹³C NMR (125 MHz, CDCl₃): δ 10.9, 17.4, 23.9, 36.1, 38.0, 44.5, 61.4, 66.2, 79.7, 81.5, 121.6, 126.5, 136.6, 138.3, 168.8; HRMS (ESI) calcd for C₁₅H₂₀O₄[M+H]⁺ m/z: 265.1440. found: 265.1433; [α]_{D}²³=−83.9° (c: 0.43 g 100 mL⁻¹, CH₂Cl₂). The 9(S) configuration of 3 was assigned based on the observed strong NOE signal (2.5%) between the 9(H) proton and 1(H) proton. 1(R),10(R)-epoxy-parthenolide (2): ¹H NMR (500 MHz, CDCl₃): δ 1.34 (s, 3H), 1.36-1.42 (m, 4H), 1.45-1.56 (m, 1H), 1.56-1.65 (m, 1H), 2.01-2.29 (m, 4H), 2.47 (dd, 1H, J=8.1, 14.0 Hz), 2.70-2.76 (m, 1H), 2.85 (d, 1H, J=12.2 Hz), 2.90 (d, 1H, J=8.9 Hz), 3.93 (t, 1H, J=8.9 Hz), 5.62 (d, 1H, J=3.0 Hz), 6.33 (d, 1H, J=3.0 Hz); ¹³C NMR (125 MHz, CDCl₃): δ 17.0, 17.5, 24.0, 26.0, 35.1, 40.1, 47.6, 60.7, 63.7, 64.6, 81.8, 101.2, 121.4, 138.8, 168.9. HRMS (ESI) calcd for C₁₅H₂₀O₄[M+H]⁺ m/z: 265.1440. found: 265.1441; [α]_{D}²³=−71.0° (c: 0.24 g 100 mL⁻¹, CH₂Cl₂).

To prepare 14-hydroxy-parthenolide (4), purified P450 variant VII-H11 (SEQ ID NO: 15) (final conc: 3 μM; 0.32 mol %) was dissolved in 400 mL 50 mM phosphate buffer (pH 8.0) in the presence of parthenolide (100 mg, final conc.: 0.95 mM), PTDH (2 μM), NADP⁺ (150 μM), and sodium phosphite (50 mM). The reaction mixture was stirred for 12 hours at 4° C. The crude product was extracted with dichloromethane (3×80 mL). The collected organic layers were dried with sodium sulfate, concentrated under vacuum, and purified by flash chromatography (hexanes/ethyl acetate: 1/2) to afford 4 (77 mg, 72%). 14-hydroxy-parthenolide (4): ¹H NMR (500 MHz, CDCl₃): δ 1.31 (s, 3H), 1.32-1.38 (m, 1H), 1.82-1.1.92 (m, 1H), 2.09-2.16 (m, 1H), 2.20-2.32 (m, 3H), 2.50 (dq, 1H, J=5.0, 13.4 Hz), 2.82-2.90 (m, 3H), 3.92 (t, 1H, J=8.7 Hz), 4.16 (d, 1H, J=11.3 Hz), 4.46 (d, 1H, J=11.8 Hz), 5.43 (dd, 1H, J=4.1, 12.4 Hz), 5.68 (d, 1H, J=3.2 Hz), 6.39 (d, 1H, J=3.8 Hz). ¹³C NMR (125 MHz, CDCl₃): δ=16.9, 23.7, 31.4, 36.3, 47.3, 59.8, 61.1, 66.2, 82.4, 121.4, 129.0, 137.8, 139.2, 169.3. HRMS (ESI) calcd for C₁₅H₂₀O₄ [M+H]⁺ m/z: 265.1440. found: 265.1440; [α]_{D}²³=−61.7° (c: 0.13 g 100 mL⁻¹, CH₂Cl₂).

### 6.3 Example 3

### Synthesis of 9-hydroxy-parthenolide and 14-hydroxy-parthenolide Using Whole-Cell Systems

This example demonstrates how whole-cell systems containing engineered P450 polypeptides provided herein, are useful for enabling the synthesis of the derivative 9-hydroxy-parthenolide and 14-hydroxy-parthenolide at preparative scales.

General Conditions for Whole-Cell Reactions:

E. coli cells (DH5α) were transformed with a pCWori-based plasmid encoding for the desired P450 under IPTG inducible promoter and a second, pAcyc-based plasmid encoding for the phosphite dehydrogenase (PTDH) enzyme under an arabinose-inducible promoter. Cells were grown in TB medium containing ampicillin (50 mg/L) and chloramphenicol (34 mg/L) until OD₆₀₀ reached 1.0. The cells were then induced with IPTG (0.2 mM) and arabinose (0.1%) and harvested after 24 hours. Cells were then resuspended in phosphate buffer and permeabilized via two cycles of freezing/thawing. Parthenolide (100 mg) and phosphite (50 mM) were added to the cell suspension, which was stirred for 12 hours. The parthenolide hydroxylation products were extracted using dichloromethane and purified via flash chromatography as described above.

E. coli cells expressing P450 variant II-05 (SEQ ID NO: 13) were utilized for the synthesis of 9(S)-hydroxy-parthenolide (3) by incubating a suspension of these cells (from 0.5 L culture) with parthenolide (100 mg). Under unoptimized conditions, 9(S)-hydroxy-parthenolide (3) was isolated from these reactions in 20% yield. Similarly, E. coli cells expressing P450 variant FL#46 (SEQ ID NO: 7) were utilized for the synthesis of 14-hydroxy-parthenolide (4) by incubating a suspension of these cells (from 0.5 L culture) with parthenolide (100 mg). Under unoptimized conditions, 14-hydroxy-parthenolide (4) was isolated from these reactions in 26% yield.

### 6.4 Example 4

### Synthesis of C9-Substituted Parthenolide Derivatives

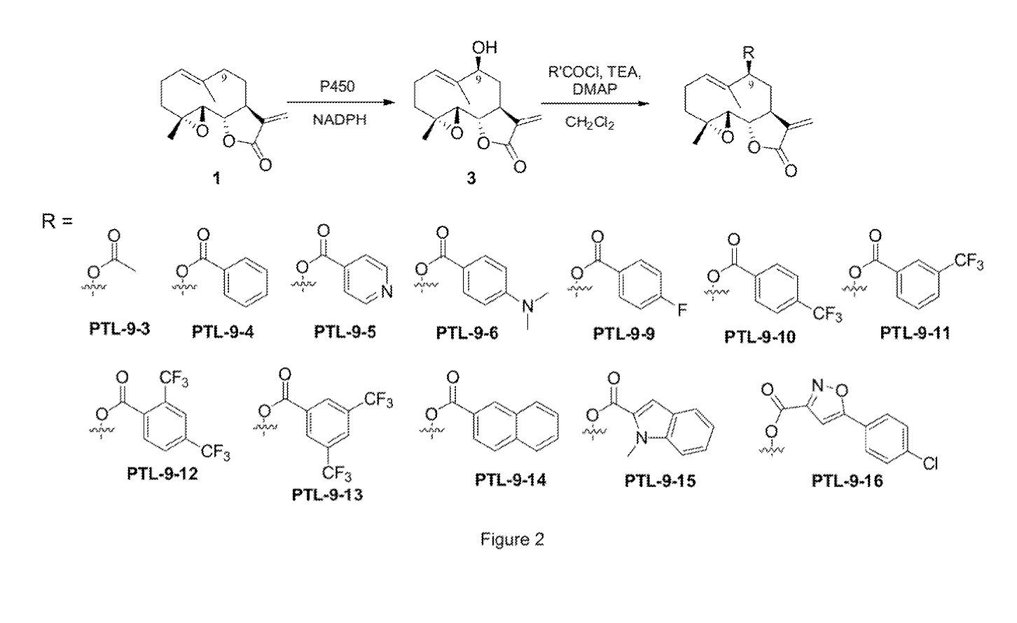

This example describes and demonstrates the preparation of compounds of general formula I according to the methods provided herein. In particular, this example illustrates how C9-substituted parthenolide analogs could be prepared by coupling selective P450-catalyzed hydroxylation of the C9 site in parthenolide followed by chemical acylation (FIG. 2).

General Conditions for Acylation of 9(S)-Hydroxy-Parthenolide:

To a solution of compound 3 in 3 mL of anhydrous dichloromethane under argon atmosphere was added 4-dimethylaminopyridine (1 equiv.), triethylamine (5 equiv.), and the corresponding acid chloride (5 equiv.). Reaction was stirred at room temperature until complete disappearance of the starting material (ca. 2 hours). At this point, the reaction mixture was added with saturated sodium bicarbonate solution (5 mL) and extracted with dichloromethane (3×5 mL). The combined organic layers were dried over sodium sulfate, concentrated under reduced pressure, and the ester product was isolated by silica gel flash chromatography (5 to 40% ethyl acetate in hexanes). Chemical structures of representative C9-substituted derivatives prepared according to the aforementioned procedure are provided in FIG. 2. Reagent concentration and characterization data for the 9-substituted parthenolide derivatives are provided below.

PTL-9-3:

Standard procedure was applied using 9(S)-hydroxy-parthenolide (6.5 mg, 0.025 mmol), 4-dimethylaminopyridine (1.5 mg, 0.0125 mmol), triethylamine (35 μL, 0.25 mmol), and acetyl chloride (9 μL, 0.125 mmol). Isolated PTL-9-003: 1.7 mg, 22% yield. ¹H NMR (500 MHz, CDCl₃): δ=1.22-1.29 (m, 1H), 1.33 (s, 3H), 1.74 (s, 3H), 2.00-2.06 (m, 1H), 2.09 (s, 3H), 2.15-2.21 (m, 2H), 2.23-2.29 (m, 1H), 2.48 (dq, 1H, J=5.31, 12.52 Hz), 2.71 (d, 1H, J=8.73 Hz), 2.91 (m, 1H), 3.86 (t, 1H, J=8.25 Hz), 5.20 (dd, 1H, J=2.23, 10.90 Hz), 5.51 (d, 1H, J=11.78 Hz), 5.69 (d, 1H, J=3.24 Hz), 6.37 (d, 1H, J=3.75); ¹³C NMR (125 MHz, CDCl₃): δ=11.7, 17.3, 21.2, 23.8, 36.0, 36.1, 44.1, 61.3, 66.0, 80.7, 81.6, 122.0, 127.8, 133.0, 138.0, 168.6, 170.0; MS (ESI) calcd for C₁₇H₂₂O₅[M+H]⁺ m/z: 307.15. found: 307.3.

PTL-9-4:

Standard procedure was applied using 9(S)-hydroxy-parthenolide (8 mg, 0.03 mmol), 4-dimethylaminopyridine (2 mg, 0.015 mmol), triethylamine (42 μL, 0.3 mmol), and benzoyl chloride (17 μL, 0.15 mmol). Isolated PTL-9-004: 5.5 mg, 50% yield. ¹H NMR (500 MHz, CDCl₃): δ=1.33-1.39 (m, 1H), 1.41 (s, 3H), 1.91 (s, 3H), 2.17-2.29 (m, 2H), 2.32-2.44 (m, 2H), 2.57 (dq, 1H, J=4.8, 12.9 Hz), 2.81 (d, 1H, J=8.9 Hz), 3.03-3.10 (m, 1H), 3.96 (t, 1H, J=8.9 Hz), 5.52 (d, 1H, J=10.1 Hz), 5.66 (d, 1H, J=12.1 Hz), 5.79 (d, 1H, J=2.4 Hz), 6.44 (d, 1H, J=2.8 Hz), 7.52 (t, 2H, J=7.7 Hz), 7.65 (t, 1H, J=7.3 Hz), 8.10 (d, 2H, J=7.2 Hz); ¹³C NMR (125 MHz, CDCl₃): δ=11.9, 17.4, 23.8, 36.0, 36.2, 44.1, 61.3, 66.1, 81.2, 81.7, 122.2, 127.9, 128.5, 129.6, 130.0, 133.1, 133.3, 138.0, 165.5, 168.6; MS (ESI) calcd for C₂₂H₂₄O₅ [M+H]⁺ m/z: 369.15. found: 369.8.

PTL-9-5:

Standard procedure was applied using 9(S)-hydroxy-parthenolide (8 mg, 0.03 mmol), 4-dimethylaminopyridine (2 mg, 0.015 mmol), triethylamine (63 μL, 0.45 mmol), and isonicotinoyl chloride (27 mg, 0.15 mmol). Isolated PTL-9-005: 3 mg, 27% yield ¹H NMR (500 MHz, CDCl₃): δ=1.34-1.40 (m, 1H), 1.42 (s, 3H), 1.90 (s, 3H), 2.20-2.30 (m, 2H), 2.33-2.44 (m, 2H), 2.56 (dq, 1H, J=5.1, 13.3 Hz), 2.80 (d, 1H, J=9.2 Hz), 3.04-3.10 (m, 1H), 3.96 (t, 1H, J=8.2 Hz), 5.54 (d, 1H, J=10.2 Hz), 5.68 (d, 1H, J=11.2 Hz), 5.77 (d, 1H, J=2.5 Hz), 6.45 (d, 1H, J=3.6 Hz), 7.90 (d, 2H, J=4.8 Hz), 8.87 (d, 2H, J=4.8 Hz); ¹³C NMR (125 MHz, CDCl₃): δ=12.0, 17.4, 24.0, 36.0, 44.1, 61.3, 66.0, 81.4, 82.4, 122.2, 123.4, 129.0, 132.2, 137.7, 138.3, 149.9, 163.7, 168.6; MS (ESI) calcd for C₂₁H₂₃NO₅ [M+H]⁺ m/z: 370.16. found: 370.4.

PTL-9-6:

Standard procedure was applied using 9(S)-hydroxy-parthenolide (10 mg, 0.037 mmol) 4-dimethylaminopyridine (2.3 mg, 0.019 mmol), triethylamine (52 μL, 0.37 mmol), and 4-(dimethylamino)benzoyl chloride (28 mg, 0.15 mmol). Isolated PTL-9-006: 9 mg, 59% yield. ¹H NMR (500 MHz, CDCl₃): δ=1.25-1.32 (m, 1H), 1.35 (s, 3H), 1.84 (s, 3H), 2.07-2.15 (m, 1H), 2.17-2.22 (m, 1H), 2.25-2.37 (m, 2H), 2.51 (dq, 1H, J=4.7, 13.0 Hz), 2.76 (d, 1H, J=8.9 Hz), 2.97-3.03 (m, 1H), 3.06 (s, 6H), 3.90 (t, 1H, J=8.1 Hz), 5.41 (d, 1H, J=11.0 Hz), 5.57 (d, 1H, J=12.0 Hz), 5.74 (d, 1H, J=3.1 Hz), 6.38 (d, 1H, J=3.9 Hz), 6.66 (d, 2H, J=8.9 Hz), 7.91 (d, 2H, J=9.1 Hz); ¹³C NMR (125 MHz, CDCl₃): δ=12.0, 17.4, 23.8, 36.1, 36.5, 40.6, 44.2, 61.7, 65.9, 80.2, 81.6, 110.7, 116.6, 122.3, 127.4, 131.2, 133.9, 138.2, 153.6, 165.8, 168.9; MS (ESI) calcd for C₂₄H₂₉NO₅[M+H]⁺ m/z: 412.20. found: 412.3.

PTL-9-9:

Standard procedure was applied using 9(S)-hydroxy-parthenolide (7 mg, 0.026 mmol), 4-dimethylaminopyridine (1.6 mg, 0.013 mmol), triethylamine (36 μL, 0.26 mmol), and 4-fluorobenzoyl chloride (15 μL, 0.13 mmol). Isolated PTL-9-009: 3 mg, 30% yield. ¹H NMR (500 MHz, CDCl₃): δ=1.33-1.38 (m, 1H), 1.41 (s, 3H), 1.90 (s, 3H), 2.17-2.29 (m, 2H), 2.32-2.42 (m, 2H), 2.57 (dq, 1H, J=5.2, 13.0 Hz), 2.81 (d, 1H, J=8.5 Hz), 3.03-3.09 (m, 1H), 3.96 (t, 1H, J=8.3 Hz), 5.50 (d, 1H, J=11.1 Hz), 5.66 (d, 1H, J=11.8 Hz), 5.78 (d, 1H, J=3.1 Hz), 6.49 (d, 1H, J=3.3 Hz), 7.19 (t, 2H, J=8.4 Hz), 8.11 (t, 2H, J=6.4 Hz); ¹³C NMR (125 MHz, CDCl₃): δ=11.9, 17.4, 23.8, 36.0, 36.2, 44.1, 61.3, 66.0, 81.4, 81.6, 115.6, 115.8, 122.15, 126.3, 128.1, 132.2 (d, J=9.41 Hz), 132.9, 137.9, 164.5, 168.4. ¹⁹F NMR (376 MHz, CDCl₃): δ=−42.49; MS (ESI) calcd for C₂₂H₂₃FO₅[M+H]⁺ m/z: 387.15. found: 387.4.

PTL-9-10:

Standard procedure was applied using 9(S)-hydroxy-parthenolide (5 mg, 0.019 mmol), 4-dimethylaminopyridine (1.2 mg, 0.0095 mmol), triethylamine (27 μL, 0.19 mmol), and the 4-(trifluoromethyl)benzoyl chloride (14 μL, 0.095 mmol). Isolated PTL-9-010: 4.4 mg, 53% yield ¹H NMR (500 MHz, CDCl₃): δ=1.34-1.39 (m, 1H), 1.42 (s, 3H), 1.90 (s, 3H), 2.21-2.30 (m, 2H), 2.33-2.44 (m, 2H), 2.58 (dq, 1H, J=4.9, 13.0 Hz), 2.81 (d, 1H, J=8.5 Hz), 3.04-3.10 (m, 1H), 3.97 (t, 1H, J=8.5 Hz), 5.54 (d, 1H, J=10.1 Hz), 5.68 (d, 1H, 11.8 Hz), 5.78 (d, 1H, J 2.8 Hz), 6.45 (d, 1H, J=2.8 Hz), 7.79 (d, 2H, J=7.7 Hz), 8.21 (d, 2H, J=8.1 Hz); ¹³C NMR (125 MHz, CDCl₃): δ=12.0, 14.1, 17.4, 22.7, 23.9, 35.9, 36.2, 44.1, 61.3, 66.1, 81.6, 81.9, 122.2, 125.6, 128.5, 130.0, 132.6, 133.2, 137.9, 164.5, 168.6; ¹⁹F NMR (376 MHz, CDCl₃): δ=−0.76; HPMS (ESI) calcd for C₂₃H₂₃F₃O₅[M+H]⁺ m/z: 437.1576. found: 437.1569.

PTL-9-11:

Standard procedure was applied using 9(S)-hydroxy-parthenolide (10 mg, 0.038 mmol), 4-dimethylaminopyridine (2.3 mg, 0.019 mmol), triethylamine (53 μL, 0.38 mmol), and 3-(trifluoromethyl)benzoyl chloride (29 μL, 0.19 mmol). Isolated PTL-9-011: 7 mg, 42% yield. ¹H NMR (400 MHz, CDCl₃): δ=1.26-1.34 (m, 1H), 1.36 (s, 3H), 1.85 (s, 3H), 2.14-2.24 (m, 2H), 2.26-2.39 (m, 2H), 2.52 (dq, 1H, J=4.9, 13.1 Hz), 2.75 (d, 1H, J=9.1 Hz), 2.97-3.05 (m, 1H), 3.91 (t, 1H, J=8.4 Hz), 5.48 (d, 1H, J=11.4 Hz), 5.62 (d, 1H, J=12.1 Hz), 5.73 (d, 1H, J=3.4 Hz), 6.40 (d, 1H, J=3.4 Hz), 7.61 (t, 1H, J=7.7 Hz), 7.85 (d, 1H, 7.7 Hz), 8.23 (d, 1H, J=7.7 Hz), 8.28 (s, 1H); ¹³C NMR (125 MHz, CDCl₃): δ=11.9, 17.4, 23.8, 36.0, 36.1, 44.1, 61.3, 66.0, 81.5, 81.9, 122.2, 126.5, 128.5, 129.2, 129.8, 130.9, 132.6, 132.8, 137.9, 164.16, 168.6; ¹⁹F NMR (376 MHz, CDCl₃): δ=−0.44; MS (ESI) calcd for C₂₃H₂₃F₃O₅ [M+Na]⁺ m/z: 459.15. found: 459.7.

PTL-9-12:

Standard procedure was applied using 9(S)-hydroxy-parthenolide (5 mg, 0.019 mmol), 4-dimethylaminopyridine (12 mg, 0.01 mmol), triethylamine (27 μL, 0.19 mmol), and 2,4(bis-trifluoromethyl)benzoyl chloride (18 μL, 0.1 mmol). Isolated PTL-9-012: 3 mg, 31% yield ¹H NMR (500 MHz, CDCl₃): δ=1.33-1.39 (m, 1H), 1.40 (s, 3H), 1.84 (s, 3H), 2.19-2.30 (m, 2H), 2.34-2.46 (m, 2H), 2.56 (dq, 1H, J=5.0, 13.0 Hz), 2.80 (d, 1H, J=8.4 Hz), 3.02-3.08 (m, 1H), 3.95 (t, 1H, J=8.4 Hz), 5.55 (d, 1H, J=10.3 Hz), 5.68 (d, 1H, J=12.6 Hz), 5.77 (d, 1H, J=2.7 Hz), 6.46 (d, 1H, J=3.4 Hz), 7.97 (s, 2H), 8.08 (s, 1H); ¹³C NMR (125 MHz, CDCl₃): δ=11.7, 17.4, 23.8, 35.5, 36.0, 44.1, 61.2, 66.0, 81.4, 83.3, 122.1, 124.2, 128.8, 130.9, 132.2, 137.7, 164.8, 168.5; ¹⁹F NMR (376 MHz, CDCl₃): δ=−0.87, 3.11; HRMS (ESI) calcd for C₂₄H₂₂F₆O₅ [M+H]⁺ m/z: 505.1453. found: 505.1450.

PTL-9-13:

Standard procedure was applied using 9(S)-hydroxy-parthenolide (9 mg, 0.034 mmol), 4-dimethylaminopyridine (2 mg, 0.017 mmol), triethylamine (47 μL, 0.34 mmol), and 3,5(bis-trifluoromethyl)benzoyl chloride (31 μL, 0.17 mmol). Isolated PTL-9-013: 5 mg, 29% yield. ¹H NMR (400 MHz, CDCl₃): δ=1.27-1.32 (m, 1H), 1.36 (s, 3H), 1.85 (s, 3H), 2.17-2.25 (m, 2H), 2.27-2.40 (m, 2H), 2.52 (dq, 1H, J=5.4, 13.0 Hz), 2.75 (d, 1H, J=8.5 Hz), 2.98-3.05 (m, 1H), 3.91 (t, 1H, J=8.1 Hz), 5.52 (d, 1H, J=10.8 Hz), 5.65 (d, 1H, J=10.8 Hz), 5.72 (s, 1H), 6.46 (s, 1H), 8.09 (s, 1H), 8.46 (s, 2H); ¹³C NMR (125 MHz, CDCl₃): δ=11.9, 17.4, 23.9, 35.9, 36.0, 44.1, 61.3, 66.0, 81.4, 82.6, 122.2, 129.1, 129.7, 132.2, 132.3, 137.8, 162.9, 168.4; ¹⁹F NMR (376 MHz, CDCl₃): δ=−0.58; HRMS (ESI) calcd for C₂₄H₂₂F₆O₅ [M+H]⁺ m/z: 505.1450. found: 505.1443.

PTL-9-14:

Standard procedure was applied using 9(S)-hydroxy-parthenolide (10 mg, 0.030 mmol), 4-dimethylaminopyridine (0.015 mmol), triethylamine (0.30 mmol), and naphthoyl chloride (0.15 mmol). Isolated: 6 mg, 37% yield. ¹H NMR (400 MHz, CDCl₃): δ=8.66 (s, 1H), 8.11-8.09 (m, 1H), 8.03 (d, J=6.4 Hz, 1H), 7.96 (d, J=6.8 Hz, 1H), 7.68 (t, J=5.6 Hz, 1H), 7.63 (t, J=6.4 Hz, 1H), 6.46 (d, J=2.8 Hz, 1H), 5.82 (d, J=2.4 Hz, 1H), 5.70 (d, J=1.6 Hz, 1H), 5.59-5.57 (m, 1H), 4.00 (t, J=7.2 Hz, 1H), 3.09 (brs, 1H), 2.84 (d, J=7.2 Hz, 1H), 2.30-2.25 (m, 5H), 1.95 (s, 3H), 1.43 (s, 3H), 1.36-1.32 (m, 2H) ppm. 2-PTL-9-15: Standard procedure was applied using 9(S)-hydroxy-parthenolide (10 mg, 0.030 mmol), 4-dimethylaminopyridine (0.015 mmol), triethylamine (0.30 mmol), and 1-methyl-1H-indole-2-carbonyl chloride (0.15 mmol). Isolated PTL-09-05: 3 mg, 18% yield. ¹H NMR (400 MHz, CDCl₃): δ=8.20 (d, J=4.4 Hz, 1H), 7.87 (d, J=3.2 Hz, 1H), 7.42-7.35 (m, 3H), 6.44 (s, 1H), 5.80 (s, 1H), 5.67 (d, J=8.8 Hz, 1H), 5.77 (d, J=8.0 Hz. 1H), 3.98-3.90 (m, 5H), 3.08 (brs, 1H), 2.87-2.81 (m, 1H), 2.58-2.10 (m, 5H), 1.93 (s, 3H), 1.42 (s, 3H), 1.41-1.32 (s, 1H) ppm. PTL-9-16: Standard procedure was applied using 9(S)-hydroxy-parthenolide (10 mg, 0.037 mmol), 4-dimethylaminopyridine (0.018 mmol), triethylamine (0.37 mmol), and 5-(4-chlorophenyl)isoxazole-3-carbonyl chloride (0.18 mmol). Isolated: 9 mg, 47% yield. ¹H NMR (400 MHz, CDCl₃): δ=7.75 (d, J=8.0 Hz, 2H), 7.48 (d, J=8.4 Hz, 2H), 6.90 (s, 1H), 6.38 (s, 1H), 5.71 (s, 1H), 5.65 (d, J=10.8 Hz, 1H), 5.52 (d, J=10.8 Hz, 1H), 3.92 (t, J=8.8 Hz, 1H), 2.99 (brs, 1H), 2.75 (d, J=8.8 Hz, 1H), 2.57-2.18 (m, 5H), 1.84 (s, 3H), 1.38-1.27 (m, 4H) ppm, 13C NMR (125 MHz, CDCl3): δ=170.8, 168.4, 158.8, 156.7, 137.7, 137.1, 132.3, 129.5, 129.0, 127.2, 124.9, 122.1, 100.2, 82.5, 81.3, 65.9, 61.3, 44.0, 35.9, 35.7, 29.6, 23.8, 17.3, 11.8 ppm.

### 6.5 Example 5

### Synthesis of C14-Substituted Parthenolide Derivatives

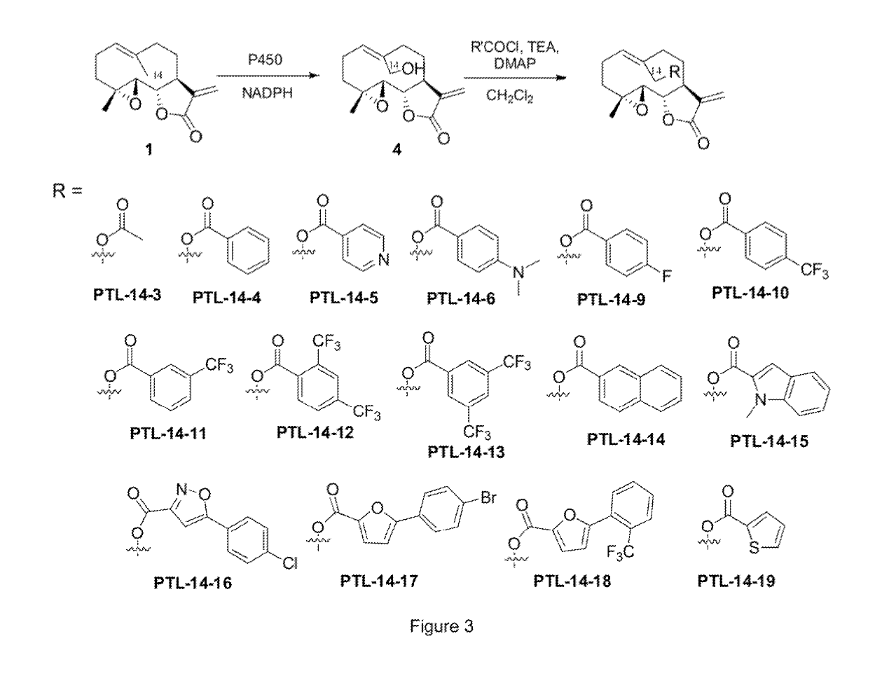

This example describes and demonstrates the preparation of compounds of general formula II according to the methods provided herein. In particular, this example illustrates how C14-substituted parthenolide analogs could be prepared by coupling selective P450-catalyzed hydroxylation of the C14 site in parthenolide followed by chemical acylation (FIG. 3).

General Conditions for Acylation of 14-hydroxy-parthenolide:

To a solution of compound 4 in 3 mL of anhydrous dichloromethane under argon atmosphere was added 4-dimethylaminopyridine (1 equiv.), triethylamine (5 equiv.), and the corresponding acid chloride (5 equiv.). Reaction was stirred at room temperature until complete disappearance of the starting material (ca. 2 hours). At this point, the reaction mixture was added with saturated sodium bicarbonate solution (5 mL) and extracted with dichloromethane (3×5 mL). The combined organic layers were dried over sodium sulfate, concentrated under reduced pressure, and the ester product was isolated by silica gel flash chromatography (5 to 40% ethyl acetate in hexanes). Chemical structures of representative C14-substituted derivatives prepared according to the aforementioned procedure are provided in FIG. 3. Reagent concentration and characterization data for the 9-substituted parthenolide derivatives are provided below.

PTL-14-3:

Standard procedure was applied using 14-hydroxy-parthenolide (3.4 mg, 0.013 mmol), 4-dimethylaminopyridine (0.8 mg, 0.0065 mmol), triethylamine (18 μL, 0.13 mmol), and acetyl chloride (5 μL, 0.065 mmol), Isolated PTL-14-003:1.3 mg, 33% yield. ¹H NMR (500 MHz, CDCl₃): δ=1.26 (s, 3H), 1.27-1.35 (m, 1H), 1.74-1.83 (m, 1H), 2.08 (s, 3H), 2.12-2.24 (m, 3H), 2.26-2.33 (m, 1H), 2.50 (dq, 1H, J=4.9, 13.4 Hz), 2.63 (dd, 1H, J=6.1, 14.0 Hz), 2.76-2.85 (m, 2H), 3.86 (t, 1H, J=8.8 Hz), 4.68 (d, 1H, J=12.1 Hz), 4.80 (d, 1H, J=12.3 Hz), 5.50 (dd, 1H, J=3.6, 12.5 Hz), 5.63 (d, 1H, J=3.2 Hz), 6.35 (d, 1H, J=3.6 Hz); ¹³C NMR (125 MHz, CDCl₃): δ=17.1, 21.1, 24.1, 31.4, 36.3, 36.8, 47.4, 61.1, 61.3, 66.3, 82.4, 121.6, 132.0, 133.5, 139.1, 169.2, 171.0; MS (ESI) calcd for C₁₇H₂₂O₅[M+H]⁺ m/z: 307.15. found: 307.1.

PTL-14-4:

Standard procedure was applied using 14-hydroxy-parthenolide (12 mg, 0.045 mmol), 4-dimethylaminopyridine (3 mg, 0.023 mmol), triethylamine (63 μL, 0.45 mmol), and benzoyl chloride (26 μL, 0.23 mmol). Isolated PTL-14-004: 3 mg, 18% yield. ¹H NMR (500 MHz, CDCl₃): δ=1.30-1.37 (4H, m), 1.81-1.90 (1H, m), 2.15-2.26 (3H, m), 2.31-2.38 (1H, m), 2.59 (1H, dq, J=5.2, 13.2 Hz), 2.75 (1H, dd, J=6.0, 14.0 Hz), 2.80 (2H, m), 3.90 (1H, t, J=8.8 Hz), 4.84 (1H, d, J=11.7 Hz), 5.07 (1H, d, J=12.1 Hz), 5.56 (1H, dd, J=4.0, 12.8 Hz), 5.63 (1H, d, J=3.3 Hz), 6.35 (1H, d, J=3.6 Hz), 7.45 (2H, t, J=7.3 Hz), 7.58 (1H, t, J=7.3 Hz), 8.05 (2H, d, J=7.7 Hz). ¹³C NMR (125 MHz, CDCl₃): δ=17.0, 24.0, 31.3, 36.2, 36.6, 47.3, 61.1, 61.6, 66.2, 82.4, 121.6, 128.6, 129.5, 129.8, 131.9, 133.3, 133.5, 139.0, 166.5, 169.1. MS (ESI) calcd for C₂₂H₂₄O₅ [M+H]⁺ m/z: 369.16. found: 369.3.

PTL-14-5:

Standard procedure was applied using 14-hydroxy-parthenolide (7 mg, 0.026 mmol), 4-dimethylaminopyridine (3 mg, 0.026 mmol), triethylamine (40 μL, 0.26 mmol), and isonicotinoyl chloride (23 mg, 0.13 mmol). Isolated PTL-14-005: 4 mg, 42% yield. ¹H NMR (500 MHz, CDCl₃): δ=1.30 (s, 3H), 1.32-1.40 (m, 1H), 1.75-1.85 (m, 1H), 2.15-2.28 (m, 3H), 2.32-2.40 (m, 1H), 2.58 (dq, 1H, J=5.8, 13.0 Hz), 2.73 (dd, 1H, J=6.1, 13.8 Hz), 2.78-2.88 (m, 2H), 3.88 (t, 1H, J=8.7 Hz), 4.82 (d, 1H, J=11.5 Hz), 4.14 (d, 1H, J=12.4 Hz), 5.60 (dd, 1H, J=4.0, 12.4 Hz), 5.64 (d, 1H, J=3.2 Hz), 6.37 (d, 1H, J=3.7 Hz), 7.84 (bs, 2H), 8.82 (bs, 2H); ¹³C NMR (125 MHz, CDCl₃): δ=17.0, 24.2, 31.2, 36.1, 36.4, 47.2, 60.9, 62.1, 66.1, 82.3, 121.7, 132.6, 132.8, 137.0, 138.8, 150.7, 165.0, 169.0; MS (ESI) calcd for C₂₁H₂₃NO₅ [M+H]⁺ m/z: 370.16. found: 370.2.

PTL-14-6:

Standard procedure was applied using 14-hydroxy-parthenolide (7 mg, 0.026 mmol), 4-dimethylaminopyridine (3 mg, 0.026 mmol), triethylamine (40 μL, 0.26 mmol), and the 4-(dimethylamino)benzoyl chloride (24 mg, 0.13 mmol). Isolated PTL-14-006: 5 mg, 47% yield. ¹H NMR (500 MHz, CDCl₃): δ=1.32 (s, 3H), 1.33-1.40 (m, 1H), 1.84-1.96 (m, 1H), 2.12-2.26 (m, 3H), 2.27-2.37 (m, 1H), 2.51-2.65 (m, 1H), 2.67-2.77 (m, 1H), 2.78-2.88 (m, 2H), 3.05 (s, 6H), 3.93 (t, 1H, J=8.8 Hz), 4.82 (d, 1H, J=12.1 Hz), 4.97 (d, 1H, J=12.4 Hz), 5.51 (dd, 1H, J=3.6, 12.7 Hz), 5.62 (d, 1H, J=3.4 Hz), 6.35 (d, 1H, J=3.6 Hz), 6.64 (d, 2H, J=9.0 Hz), 7.86 (d, 2H, J=8.9 Hz); ¹³C NMR (125 MHz, CDCl₃): δ=16.9, 24.0, 31.6, 36.3, 36.9, 40.0, 47.3, 61.2, 66.2, 82.5, 110.8, 116.2, 121.5, 131.2, 132.6, 134.0, 139.0, 153.4, 166.8, 169.2; MS (ESI) calcd for C₂₄H₂₉NO₅[M+H]⁺ m/z: 412.20. found: 412.3.

PTL-14-9:

Standard procedure was applied using 14-hydroxy-parthenolide (14 mg, 0.053 mmol), 4-dimethylaminopyridine (3 mg, 0.027 mmol), triethylamine (74 μL, 0.53 mmol), and 4-fluorobenzoyl chloride (31 μL, 0.27 mmol). Isolated PTL-14-009: 5.6 mg, 27% yield. ¹H NMR (500 MHz, CDCl₃): δ=1.30 (s, 3H), 1.31-1.38 (m, 1H), 1.76-1.87 (m, 1H), 2.13-2.26 (m, 3H), 2.30-2.38 (m, 1H), 2.57 (dq, 1H, J=5.3, 13.3 Hz), 2.73 (dd, 1H, J=6.2, 13.5 Hz), 2.78-2.87 (m, 2H), 3.88 (t, 1H, J=8.8 Hz), 4.80 (d, 1H, J=12.4 Hz), 5.07 (d, 1H, J=11.8 Hz), 5.56 (dd, 1H, J=3.6, 12.4 Hz), 5.62 (d, 1H, J=4.1 Hz), 6.35 (d, 1H, J=3.5 Hz), 7.12 (t, 2H, J=8.5 Hz), 8.02 (dt, 2H, J=3.3, 5.4 Hz); ¹³C NMR (125 MHz, CDCl₃): δ=17.0, 24.0, 31.2, 36.2, 36.5, 47.3, 61.0, 61.5, 66.2, 82.3, 115.6, 115.9, 121.6, 132.1, 132.2, 133.4, 138.9, 165.6, 169.0; ¹⁹F NMR (376 MHz, CDCl₃): δ=−42.45; MS (ESI) calcd for C₂₂H₂₃FO₅ [M+H]⁺ m/z: 387.15. found: 387.5.

PTL-14-10:

Standard procedure was applied using 14-hydroxy-parthenolide (16 mg, 0.061 mmol), 4-dimethylaminopyridine (3.7 mg, 0.03 mmol), triethylamine (85 μL, 0.61 mmol), and 4-(trifluoromethyl)benzoyl chloride (45 μL, 0.3 mmol). Isolated PTL-14-010: 7 mg, 26% yield. ¹H NMR (500 MHz, CDCl₃): δ=1.31 (s, 3H), 1.32-1.39 (m, 1H), 1.77-1.87 (m, 1H), 2.19-2.28 (m, 3H), 2.33-2.39 (m, 1H), 2.60 (dq, 1H, J=5.5, 13.3 Hz), 2.74 (dd, 1H, J=5.1, 13.7 Hz), 2.79-2.88 (m, 2H), 3.89 (t, 1H, J=8.6 Hz), 4.84 (d, 1H, J=12.1 Hz), 5.13 (d, 1H, J=12.1 Hz), 5.60 (dd, 1H, J=4.3, 12.1 Hz), 5.63 (d, 1H, J=2.7 Hz), 6.36 (d, 1H, J=3.5 Hz), 7.73 (d, 2H, J=8.2 Hz), 8.13 (d, 2H, J=8.2 Hz); ¹³C NMR (125 MHz, CDCl₃): δ=17.0, 24.1, 31.2, 36.1, 36.4, 47.3, 61.0, 61.9, 66.1, 82.3, 121.6, 125.6, 130.0, 132.4, 133.0, 138.9, 165.3, 169.0; ¹⁹F NMR (376 MHz, CDCl₃): δ=−0.77; HRMS (ESI) calcd for C₂₃H₂₃F₃O₅ [M+H]⁺ m/z: 437.1576. found: 437.1572.

PTL-14-11:

Standard procedure was applied using 14-hydroxy-parthenolide (13 mg, 0.049 mmol), 4-dimethylaminopyridine (3 mg, 0.025 mmol), triethylamine (68 μL, 0.49 mmol), and 3-(trifluoromethyl)benzoyl chloride (37 μL, 0.025 mmol). Isolated PTL-14-011: 10 mg, 38% yield. ¹H NMR (500 MHz, (CD₃)₂CO): δ=1.36-1.41 (m, 4H), 2.02-2.07 (m, 1H), 2.23 (dd, 1H, J=5.0, 12.4 Hz), 2.29-2.39 (m, 3H), 2.70-2.81 (m, 2H), 3.03 (d, 1H, J=9.4 Hz), 3.10-3.16 (m, 1H), 4.13 (t, 1H, J=8.4 Hz), 5.02 (d, 1H, J=11.9 Hz), 5.29 (d, 1H, J=11.9 Hz), 5.78 (d, 1H, J=3.0 Hz), 5.84 (dd, 1H, J=3.5, 12.9 Hz), 6.21 (d, 1H, J=4.0 Hz), 7.85 (t, 1H, J=7.9 Hz), 8.06 (d, 1H, J=7.9 Hz), 8.32 (s, 1H), 8.36 (d, 1H, J=7.4 Hz); ¹³C NMR (125 MHz, (CD₃)₂CO): δ=17.42, 24.8, 31.9, 37.1, 47.8, 61.6, 63.1, 66.8, 83.4, 120.8, 126.9, 130.7, 131.1, 132.4, 133.1, 134.1, 134.6, 141.5, 165.7, 170.0; ¹⁹F NMR (376 MHz, CDCl₃): δ 0.46; MS (ESI) calcd for C₂₃H₂₃F₃O₅ [M+Na]⁺ m/z: 459.15. found: 459.7.

PTL-14-12:

Standard procedure was applied using 14-hydroxy-parthenolide (27 mg, 0.10 mmol), 4-dimethylaminopyridine (6 mg, 0.051 mmol), triethylamine (140 μL, 0.1 mmol), and 2,4(bis-trifluoromethyl)benzoyl chloride (90 μL, 0.5 mmol). Isolated PTL-14-012: 20 mg, 40% yield. ¹H NMR (500 MHz, (CD₃)₂CO): δ=1.30-1.40 (m, 4H), 1.92-2.00 (m, 1H), 2.18 (dd, 1H, J=4.9, 12.5 Hz), 2.22-2.34 (m, 3H), 2.65-2.75 (m, 2H), 2.96 (d, 1H, J=9.0 Hz), 3.04-3.10 (m, 1H), 4.05 (t, 1H, J=8.7 Hz), 4.96 (d, 1H, J=11.9 Hz), 5.28 (d, 1H, J=11.9 Hz), 5.74 (d, 1H, J=3.2 Hz), 5.81 (dd, 1H, J=3.8, 12.5 Hz), 6.17 (d, 1H, J=3.5 Hz), 8.10 (d, 1H, J=9.1 Hz), 8.19 (s, 2H); ¹³C NMR (125 MHz, (CD₃)₂CO): δ=17.4, 24.7, 31.6, 36.9, 37.0, 47.7, 61.5, 63.9, 66.7, 83.3, 120.7, 124.9, 130.7, 132.2, 133.5, 134.0, 136.1, 141.4, 166.2, 169.9; ¹⁹F NMR (376 MHz, CDCl₃): δ=−0.88, 2.66; MS (ESI) calcd for C₂₄H₂₂F₆O₅ [M+Na]⁺ m/z: 527.14. found: 527.7.

PTL-14-13:

Standard procedure was applied using 14-hydroxy-parthenolide (15 mg, 0.057 mmol), 4-dimethylaminopyridine (3.5 mg, 0.029 mmol), triethylamine (80 μL, 0.57 mmol), and the 3,5(bis-trifluoromethyl)benzoyl chloride (51 μL, 0.29 mmol). Isolated PTL-14-013: 7 mg, 24% yield. ¹H NMR (500 MHz, (CD₃)₂CO): δ=1.30-1.38 (m, 4H), 1.94-2.02 (m, 1H), 2.15-2.22 (m, 1H), 2.23-2.36 (m, 3H), 2.67-2.83 (m, 2H), 2.98 (d, 1H, J=8.4 Hz), 3.05-3.12 (m, 1H), 4.07 (t, 1H, J=8.4 Hz), 5.01 (d, 1H, J=11.2 Hz), 5.32 (d, 1H, J=12.1 Hz), 5.74 (s, 1H), 5.82 (d, 1H, J=12.1 Hz), 6.71 (s, 1H), 8.36 (s, 1H), 8.54 (s, 2H); ¹³C NMR (125 MHz, (CD₃)₂CO): δ=17.4, 24.8, 31.7, 36.9, 37.0, 47.8, 61.5, 63.4, 66.7, 83.3, 120.7, 123.1, 125.2, 127.6, 130.6, 132.7, 133.0, 133.5, 133.9, 134.3, 141.4, 164.5, 169.8; ¹⁹F NMR (376 MHz, CDCl₃): δ=−0.59; HRMS (ESI) calcd for C₂₄H₂₂F₆O₅ [M+H]⁺ m/z: 505.1450. found: 505.1454.

PTL-14-14:

Standard procedure was applied using 9(S)-hydroxy-parthenolide (8 mg, 0.030 mmol), 4-dimethylaminopyridine (0.015 mmol), triethylamine (0.30 mmol), and 2-naphthoyl chloride (0.15 mmol). Isolated: 5 mg, 40% yield. ¹H NMR (400 MHz, CDCl₃): δ=8.57 (s, 1H), 8.02 (d, J=8.8 Hz, 1H), 7.94-7.87 (m, 3H), 7.62-7.55 (m, 2H), 6.36 (d, J=3.2 Hz, 1H), 5.63 (d, J=3.2 Hz, 1H), 5.61 (dd, J=4.0, 8.4 Hz, 1H), 5.15 (d, J=12 Hz, 1H), 4.90 (d, J=12.0 Hz, 1H), 3.95 (t, J=8.4 Hz, 1H), 2.86-2.77 (m, 3H), 2.72-2.61 (m, 1H), 2.38-2.35 (m, 1H), 2.27-2.18 (m, 3H), 1.95-1.87 (m, 1H), 1.39-1.31 (m, 4H) ppm, ¹³C NMR (125 MHz, CDCl₃): δ=169.0, 166.6, 138.9, 135.6, 133.5, 132.4, 131.9, 131.1, 129.3, 128.5, 128.4, 127.8, 126.9, 126.8, 124.9, 121.5, 82.4, 66.2, 61.6, 61.0, 47.3, 36.6, 36.2, 31.2, 24.0, 17.0 ppm.

PTL-14-15:

Standard procedure was applied using 14-hydroxy-parthenolide (7 mg, 0.026 mmol), 4-dimethylaminopyridine (0.013 mmol), triethylamine (0.26 mmol), and 1-methyl-1H-indole-2-carbonyl chloride (0.13 mmol). Isolated: 4 mg, 35% yield. ¹H NMR (400 MHz, CDCl₃): δ=8.13 (d, J=7.6 Hz, 1H), 7.74 (s, 1H), 7.37-7.27 (m, 3H), 6.34 (d, J=3.6 Hz, 1H), 5.61 (d, J=2.8 Hz, 1H), 5.55 (dd, J=4.0, 9.6 Hz, 1H), 5.10 (d, J=12 Hz, 1H), 4.85 (d, J=12 Hz, 1H), 3.93 (t, J=8.8 Hz, 1H), 3.84 (s, 3H), 2.82-2.78 (m, 3H), 2.64-2.59 (m, 1H), 2.36-2.13 (m, 4H), 1.88-1.84 (m, 1H), 1.33 (s, 3H), 1.30-1.08 (m, 1H) ppm, ¹³C NMR (125 MHz, CDCl₃): δ=178.2, 169.1, 151.1, 139.0, 137.2, 135.1, 134.2, 131.2, 123.0, 122.1, 116.1, 109.9, 82.4, 66.2, 61.1, 59.9, 47.3, 36.5, 36.2, 33.5, 31.1, 23.9, 17.0 ppm.

PTL-14-16:

Standard procedure was applied using 14-hydroxy-parthenolide (10 mg, 0.037 mmol), 4-dimethylaminopyridine (0.018 mmol), triethylamine (0.37 mmol), and 5-(4-chlorophenyl)isoxazole-3-carbonyl chloride (0.18 mmol). Isolated: 9 mg, 47% yield. ¹H NMR (400 MHz, CDCl₃): δ=7.73 (d, J=8.4 Hz, 2H), 7.48 (d, J=8.8 Hz, 2H), 6.90 (s, 1H), 6.35 (s, 1H), 5.62 (s, 1H), 5.58 (brs, 1H), 5.08 (d, J=12 Hz, 1H), 4.93 (d, J=12 Hz, 1H), 4.02 (t, J=8.4 Hz, 1H), 2.84-2.79 (m, 2H), 2.73-2.70 (m, 1H), 2.61-2.52 (m, 1H), 2.36-2.34 (m, 1H), 2.25-2.19 (m, 3H), 2.07-1.95 (m, 1H), 1.38-1.33 (m, 1H), 1.24 (s, 3H) ppm, 13C NMR (125 MHz, CDCl3): δ=170.8, 169.1, 159.8, 156.5, 138.9, 137.1, 133.1, 132.3, 129.5, 127.1, 124.8, 121.5, 100.1, 82.1, 66.3, 62.9, 61.0, 47.3, 36.9, 36.1, 31.3, 29.6, 24.2, 16.9 ppm.

PTL-14-17:

Standard procedure was applied using 14-hydroxy-parthenolide (10 mg, 0.037 mmol), 4-dimethylaminopyridine (0.018 mmol), triethylamine (0.37 mmol), and 5-(4-bromophenyl)furan-2-carbonyl chloride (0.018 mmol). Isolated: 6 mg, 31% yield. ¹H NMR (400 MHz, CDCl₃): δ=7.57-7.52 (m, 4H), 7.24 (d, J=3.2 Hz, 1H), 6.74 (d, J=3.6 Hz, 1H), 6.37 (d, J=3.2 Hz, 1H), 5.63 (d, J=2.4 Hz, 1H), 5.58 (dd, J=3.6, 8.8 Hz, 1H), 4.98 (d, J=12.0 Hz, 1H), 4.90 (d, J=12 Hz, 1H), 4.0 (t, J=8.4 Hz, 1H), 2.85-2.81 (m, 2H), 2.71-2.68 (m, 1H), 2.55-2.47 (m, 1H), 2.35-2.32 (m, 1H), 2.25-2.20 (m, 3H), 2.07-1.98 (m, 1H), 1.37-1.29 (m, 4H) ppm, ¹³C NMR (125 MHz, CDCl₃): δ=169.0, 158.4, 156.8, 143.4, 139.0, 132.9, 132.5, 132.1, 128.1, 126.1, 123.4, 121.5, 120.5, 107.4, 82.2, 66.2, 62.0, 61.1, 47.3, 37.0, 36.2, 31.6, 29.7, 24.1, 16.9 ppm.

PTL-14-18:

Standard procedure was applied using 14-hydroxy-parthenolide (10 mg, 0.037 mmol), 4-dimethylaminopyridine (0.018 mmol), triethylamine (0.37 mmol), and 5-(2-(trifluoromethyl)phenyl)furan-2-carbonyl chloride (0.18 mmol). Isolated: 7 mg, 37% yield. ¹H NMR (400 MHz, CDCl₃): δ=7.78 (d, J=7.6 Hz, 1H), 7.71 (d, J=7.6 Hz, 1H), 7.62 (t, J=8.0 Hz, 1H), 7.53 (t, J=6.8 Hz, 1H), 7.28-7.25 (m, 1H), 6.78 (s, 1H), 6.32 (d, J=2.8 Hz, 1H), 5.59-5.54 (m, 2H), 4.99 (d, J=12 Hz, 1H), 4.89 (d, J=12 Hz, 1H), 3.97 (t, J=8.8 Hz, 1H), 2.82-2.79 (m, 1H), 2.71-2.68 (m, 1H), 2.59-2.45 (m, 1H), 2.34-2.32 (m, 1H), 2.24-2.18 (m, 3H), 2.03-1.97 (m, 2H), 1.38-1.28 (m, 4H) ppm, 13C NMR (125 MHz, CDCl3): δ=169.1, 158.4, 154.3, 139.0, 133.0, 132.4, 132.0, 130.5, 129.3, 126.8, 121.4, 120.0, 114.7, 112.1, 82.2, 66.2, 62.2, 61.1, 47.3, 46.0, 37.1, 36.2, 31.5, 29.7, 24.2, 16.9 ppm.

PTL-14-19:

Standard procedure was applied using 14-hydroxy-parthenolide (8 mg, 0.030 mmol), 4-dimethylaminopyridine (0.015 mmol), triethylamine (0.30 mmol), and thiophene-2-carbonyl chloride (0.15 mmol). Isolated: 6 mg, 53% yield. ¹H NMR (400 MHz, CDCl₃): δ=7.80 (d, J=3.6 Hz, 1H), 7.57 (d, J=4.8 Hz, 1H), 7.12 (t, J=4.8 Hz, 1H), 6.35 (d, J=3.2 Hz, 1H), 5.63 (d, J=2.8 Hz, 1H), 5.56 (dd, J=3.6, 8.8 Hz, 1H), 4.98 (d, J=12 Hz, 1H), 4.85 (d, J=12 Hz, 1H), 3.97 (t, J=8.8 Hz, 1H), 2.84-2.80 (m, 2H), 2.69-2.78 (m, 1H), 2.63-2.52 (m, 1H), 2.41-2.32 (m, 3H), 2.24-2.17 (m, 1H), 2.01-1.82 (m, 1H), 1.33-1.29 (m, 4H) ppm, ¹³C NMR (125 MHz, CDCl₃): δ=169.1, 161.9, 139.0, 133.9, 133.1, 132.9, 132.7, 132.2, 128.0, 121.5, 66.2, 62.3, 61.0, 47.2, 36.9, 36.2, 31.7, 29.7, 24.1, 16.9 ppm.\

### 6.6 Example 6

### Synthesis of C9- and C14-Substituted Parthenolide Derivatives Via Other Hydroxyl Group Functionalization Methods

This example further demonstrates the preparation of compounds of general formula I and II according to the methods provided herein. In particular, this example illustrates how C9- and C14-substituted parthenolide analogs could be prepared by coupling selective P450-catalyzed hydroxylation of the C9 or C14 site in parthenolide followed by chemical functionalization/interconversion of the enzymatically installed hydroxyl group (—OH).

Beside acylation as shown in the Examples 4 and 5, other chemistries for functionalization/interconversion of an hydroxyl group can be coupled to P450-catalyzed parthenolide hydroxylation in order to obtain C9- or C14-substituted parthenolide derivatives according to the invention. These additional chemical methods include, but are not limited to, —OH group alkylation, Mitsunobu substitution, (metal-catalyzed) carbene insertion, and deoxyhalogenation.

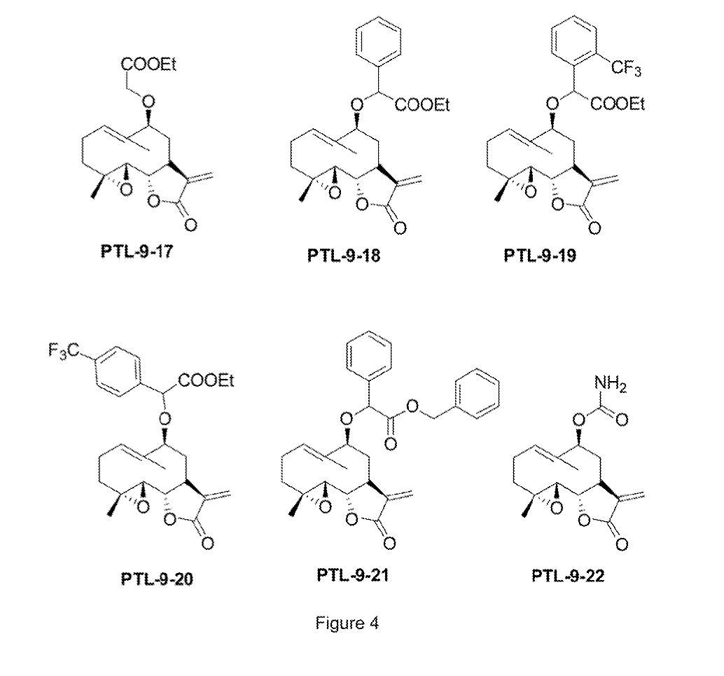

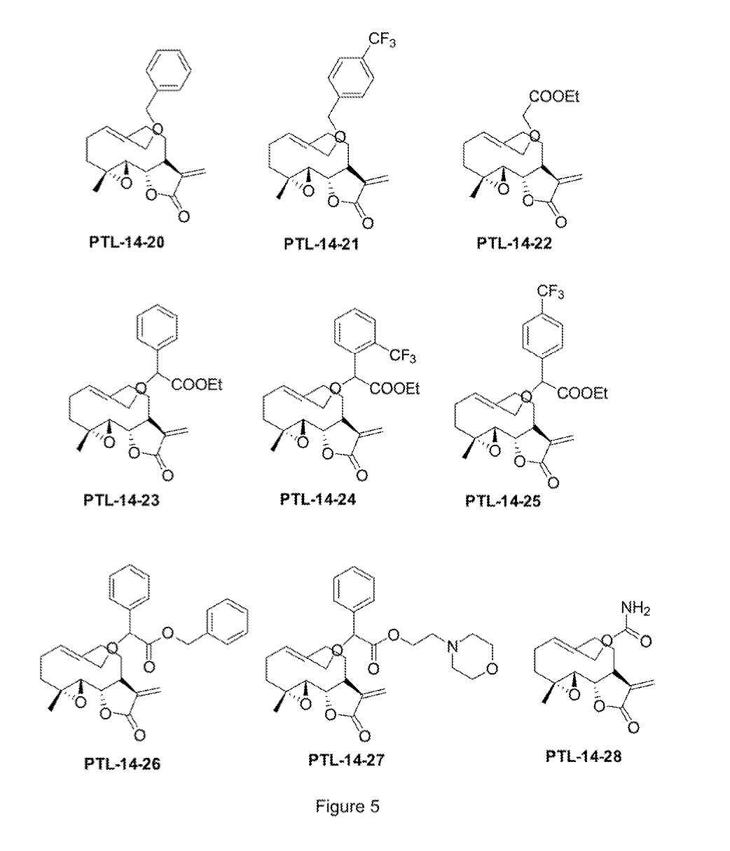

For example, alcohols can be converted to ether derivatives via transition metal-catalyzed carbene O—H insertion. (Cox, Kulagowski et al. 1992; Peddibhotla, Dang et al. 2007) Accordingly, various C9-modified parthenolide derivatives such as compound PTL-9-17 through PTL-9-21 in FIG. 4 could be readily obtained via reaction of the enzymatically produced 9(S)-hydroxy-parthenolide (3) with a desired diazo compound (e.g., ethyl diazoacetate for preparation of PTL-9-17) in the presence of a rhodium catalyst (e.g., Rh₂(OAc)₄). Similarly, various C14-modified parthenolide derivatives such as compound PTL-14-22 through PTL-9-27 in FIG. 5 could be readily obtained starting from 14-hydroxy-parthenolide using analogous synthetic procedures. These studies indicate that a variety of diazo reagents can be utilized for the purpose of preparing parthenolide analogs within the scope of the invention.

Another well established method for conversion an alcohol to an ether derivative is through direct alkylation. As illustrated by PTL-14-20 and PTL-14-21 (FIG. 5), C14-substituted parthenolide derivatives could be readily obtained via alkylation of enzymatically prepared 14-hydroxy-parthenolide with the corresponding alkyl halide. These studies indicate that a variety of alkyl halides and substituted derivatives thereof can be utilized for the purpose of preparing parthenolide analogs within the scope of the invention.

An established strategy for converting an alcohol to a carbamate derivative (i.e. R—OH→R—O(CO)NH—R′) involves reacting the alcohol with a desired isocyanate reagent (e.g., aryl or alkyl isocyanate compound). Accordingly, as illustrated by the successful preparation of PTL-9-22 (FIG. 4) and PTL-14-28 (FIG. 5), C9- and C14-carbamate derivarives of parthenolide could be readily afforded upon reaction of the enzymatically produced 9(S)-hydroxy-parthenolide (3) and 14-hydroxy-parthenolide (4), respectively, with a isocyanate reagent. These studies indicate that a variety of isocyanate reagents can be utilized for the purpose of preparing parthenolide analogs within the scope of the invention.

General Conditions for Derivatization of 9(S)-hydroxy-parthenolide and 14-hydroxy-parthenolide via Rhodium-Catalyzed O—H Functionalization:

9(S)-hydroxy-parthenolide or 14-hydroxy-parthenolide (1 mmol) and Rh₂(OAc)₄ (5 mol %) were added to 2 mL of dichloromethane and the mixture was stirred at room temperature under argon atmosphere. To this solution was added the desired diazo compound (2 mmol) in 2 mL of dichloromethane drop wise over 15-20 minutes, and the mixture was then stirred for additional 2 h. After completion of the reaction, the mixture was filtered through celite and washed with CH₂Cl₂. The solvent was removed under reduced pressure, and the crude product was purified by flash chromatography.

General Conditions for Derivatization of 14-hydroxy-parthenolide Via Alkylation:

A mixture of 14-hydroxy-parthenolide (1 mmol) and Ag₂O (2 mmol) in THF was stirred at room temperature under an argon atmosphere. To this solution was added benzyl bromide (1.5 mmol) and the mixture was then stirred for 24 h. The solvent was removed under reduced pressure, and the crude product was purified by flash chromatography.

General Conditions for Carbamate Derivatization of 9(S)-hydroxy-parthenolide and 14-hydroxy-parthenolide:

To a solution of 9(S)-hydroxy-parthenolide or 14-hydroxy-parthenolide (1 mmol) in dichloromethane at 0° C., trichloroacetyl isocyanate (1.2 mmol) was added. The reaction was stirred at 0° C. for 30 min and then neutral aluminium oxide was added. The mixture was stirred for 3 hrs at room temperature and after completion of the reaction, mixture was filtered through celite and subsequently washed with dichloromethane. The solvent was removed under reduced pressure, and the crude product was purified by flash chromatography.

PTL-9-17:

Standard procedure was applied using 9(S)-hydroxy-parthenolide (5 mg, 0.018 mmol), Rh₂(OAc)₄ (0.4 mg, 5 mol %) and ethyldiazoacetate (4 mg, 0.36 mmol). Isolated: 2 mg, 35% yield. ¹H NMR (400 MHz, CDCl₃): δ=6.36 (d, J=3.2 Hz, 1H), 5.73 (d, J=3.2 Hz, 1H), 5.45 (d, J=10 Hz, 1H), 4.23-4.18 (m, 3H), 4.02-3.82 (m, 3H), 2.85-2.80 (m, 1H), 2.68 (d, J=8.8 Hz, 1H), 2.41-2.37 (m, 1H), 2.27-2.16 (m, 3H), 2.05-1.98 (m, 2H), 1.76 (s, 3H), 1.41-1.27 (m, 6H) ppm, ¹³C NMR (125 MHz, CDCl3): δ=170.2, 168.7, 138.0, 129.9, 121.8, 86.8, 81.2, 66.1, 64.7, 61.3, 60.9, 44.8, 36.1, 36.5, 24.1, 22.7, 17.4, 14.2, 11.1 ppm.

PTL-9-18:

Standard procedure was applied using 9(S)-hydroxy-parthenolide (10 mg, 0.037 mmol), Rh₂(OAc)₄ (0.8 mg, 5 mol %) and phenylethyldiazoacetate (14 mg, 0.74 mmol). Isolated: 5 mg, 32% yield. ¹H NMR (400 MHz, CDCl3): δ=7.41-7.33 (m, 10H), 6.37 (s, 1H), 6.31 (s, 1H), 5.78 (brs, 1H), 5.62 (s, 1H), 5.49-5.47 (m, 2H), 4.73 (s, 2H), 4.19-4.08 (m, 5H), 3.67-3.64 (m, 2H), 3.67-3.64 (m, 1H), 2.88 (brs, 1H), 2.77-2.70 (m, 2H), 2.57-2.48 (m, 4H), 2.32-1.97 (m, 7H), 1.65 (s, 3H), 1.60 (s, 3H), 1.33 (s, 6H), 1.22-1.10 (m, 8H) ppm.

PTL-9-19:

Standard procedure was applied using 9(S)-hydroxy-parthenolide (5 mg, 0.018 mmol), Rh₂(OAc)₄ (0.4 mg, 5 mol %) and ethyl 2-diazo-2-(2-(trifluoromethyl)-phenyl)-acetate (9.5 mg, 0.37 mmol), Isolated: 2.7 mg, 28% yield. ¹H NMR (400 MHz, CDCl₃): δ=7.82-7.47 (m, 8H), 6.39-6.31 (m, 2H), 5.79 (brs, 1H), 5.56-5.31 (m, 3H), 5.16 (s, 2H), 4.13 (brs, 4H), 3.84-3.65 (m, 3H), 2.97-2.88 (m, 4H), 2.68-2.48 (m, 4H), 2.26-2.03 (m, 7H), 1.63 (s, 6H), 1.34-1.15 (m, 14H) ppm.

PTL-9-20:

Standard procedure was applied using 9(S)-hydroxy-parthenolide (10 mg, 0.037 mmol), Rh₂(OAc)₄ (0.8 mg, 5 mol %) and ethyl 2-diazo-2-(4-(trifluoromethyl)-phenyl)-acetate (19 mg, 0.74 mmol), Isolated: 8 mg, 42% yield. ¹H NMR (400 MHz, CDCl₃): δ=7.66-7.53 (m, 8H), 6.38 (d, J=3.2 Hz, 1H), 6.33 (d, J=3.2 Hz, 1H), 5.79 (d, J=2.8 Hz, 1H), 5.63 (d, J=2.4 Hz, 1H), 5.50-5.48 (m, 1H), 5.28 (brs, 1H), 4.78 (s, 2H), 4.19-4.02 (m, 8H), 3.87-3.83 (m, 2H), 3.64-3.62 (m, 1H), 2.88 (brs, 1H), 2.69-2.67 (m, 2H), 2.57-2.49 (m, 4H), 2.33-1.83 (m, 4H), 1.73 (s, 3H), 1.62 (s, 3H), 1.33-1.17 (m, 14H) ppm.

PTL-9-21:

Standard procedure was applied using 9(S)-hydroxy-parthenolide (10 mg, 0.037 mmol), Rh₂(OAc)₄ (0.8 mg, 5 mol %) and benzyl 2-diazo-2-phenylacetate (19 mg, 0.74 mmol), Isolated: 6 mg, 32% yield. ¹H NMR (400 MHz, CDCl3): δ=7.42-7.27 (m, 20H), 6.34 (s, 1H), 6.31 (s, 1H), 5.68 (s, 1H), 5.61 (s, 1H), 5.28-5.08 (m, 5H), 4.79 (brs, 2H), 3.82-3.64 (m, 4H), 2.70-2.03 (m, 18H), 1.69 (s, 3H), 1.47 (s, 3H), 1.32 (s, 3H), 1.20 (s, 3H) ppm.

PTL-9-22.

Standard procedure was applied using 9-hydroxy-parthenolide (8 mg, 0.030 mmol), 4-dimethylaminopyridine (0.015 mmol), triethylamine (0.30 mmol), and trichloroacetyl isocyanate (0.15 mmol). Isolated: 6 mg, 53% yield. ¹H NMR (400 MHz, CDCl₃): δ=6.37 (d, J=2.8 Hz, 1H), 5.71 (d, J=2.4 Hz, 1H), 5.51 (d, J=8.0 Hz, 1H), 5.12 (d, J=8.0 Hz, 1H), 4.65 (brs, 2H), 3.86 (t, J=6.8 Hz, 1H), 2.93-2.90 (m, 1H), 2.71 (d, J=7.2 Hz, 1H), 2.49-2.46 (m, 1H), 2.27-2.15 (m, 3H), 2.04-1.97 (m, 1H), 1.73 (s, 3H), 1.34 (s, 3H), 1.32-1.27 (m, 1H) ppm.

PTL-14-20:

Standard procedure was applied using 14-hydroxy-parthenolide (10 mg, 0.037 mmol), Ag₂O (18 mg, 0.074) and benzyl bromide (6 mg, 0.56 mmol). Isolated: 4 mg, 29% yield. ¹H NMR (400 MHz, CDCl3): δ=7.34-7.25 (m, 5H), 6.33 (d, J=3.2 Hz, 1H), 5.61 (d, J=2.8 Hz, 1H), 5.43-5.40 (m, 1H), 4.53 (d, J=11.6 Hz, 1H), 4.43 (d, J=11.6 Hz, 1H), 4.09-4.02 (m, 2H), 3.87 (t, J=8.8 Hz, 1H), 2.78-2.72 (m, 3H), 2.44-2.39 (m, 1H), 2.22-2.04 (m, 4H), 1.87-1.83 (m, 1H), 1.24-1.21 (m, 4H) ppm, ¹³C NMR (125 MHz, CDCl₃): δ=169.2, 139.2, 137.8, 135.6, 129.9, 128.4, 127.8, 127.7, 121.3, 82.3, 72.4, 67.1, 66.2, 61.1, 47.3, 37.0, 36.3, 31.5, 29.7, 23.8, 16.9 ppm.

PTL-14-21:

Standard procedure was applied using 14-hydroxy-parthenolide (10 mg, 0.037 mmol), Ag₂O (18 mg, 0.074) and substituted benzyl bromide (9 mg, 0.56 mmol), Isolated: 6 mg, 37% yield. ¹H NMR (400 MHz, CDCl₃): δ=7.61 (d, J=8.0 Hz, 2H), 7.42 (d, J=8.0 Hz, 2H), 6.34 (d, J=3.2 Hz, 1H), 5.62 (d, J=2.4 Hz, 1H), 5.47 (d, J=8.8 Hz, 1H), 4.58 (d, J=12.4 Hz, 1H), 4.49 (d, J=12.4 Hz, 1H), 4.13-4.06 (m, 2H), 3.87 (t, J=8.8 Hz, 1H), 2.79 (m, 3H), 2.44-2.36 (m, 1H), 2.25-2.03 (m, 4H), 1.88-1.79 (m, 1H), 1.37-1.22 (m, 4H) ppm, 13C NMR (125 MHz, CDCl₃): δ=169.1, 142.0, 139.1, 135.2, 130.3, 127, 125.4, 121.4, 82.3, 71.5, 67.5, 66.2, 61.0, 47.3, 36.8, 36.3, 31.5, 29.7, 23.8, 16.9 ppm.

PTL-14-22:

Standard procedure was applied using 14-hydroxy-parthenolide (5 mg, 0.018 mmol), Rh2(OAc)4 (0.4 mg, 5 mol %) and ethyldiazoacetate (4 mg, 0.36 mmol). Isolated: 3 mg, 45% yield. 1H NMR (400 MHz, CDCl3): δ=6.33 (d, J=4.0 Hz, 1H), 5.62 (brs, 1H), 5.48 (d, J=12 Hz, 1H), 4.24-3.98 (m, 6H), 3.91 (t, J=8.0 Hz, 1H), 2.80-2.78 (m, 3H), 2.50-2.44 (m, 1H), 2.26-2.05 (m, 4H), 2.00-1.89 (m, 1H), 1.30-1.09 (m, 7H) ppm, ¹³C NMR (125 MHz, CDCl3): δ=170.2, 169.2, 139.2, 134.9, 130.9, 121.3, 82.2, 68.3, 67.3, 66.2, 61.1, 60.9, 47.4, 37.0, 36.3, 31.5, 23.8, 16.9, 14.2 ppm.

PTL-14-23:

Standard procedure was applied using 14-hydroxy-parthenolide (10 mg, 0.037 mmol), Rh₂(OAc)₄ (0.8 mg, 5 mol %) and phenylethyldiazoacetate (14 mg, 0.74 mmol). Isolated: 6 mg, 38% yield. ¹H NMR (400 MHz, CDCl₃): δ=7.38-7.35 (m, 10H), 6.32 (d, J=3.2, 2H), 5.28 (brs, 2H), 5.59-5.46 (m, 2H), 5.43 (s, 1H), 4.84 (s, 1H), 4.80-4.02 (m, 8H), 3.92 (t, J=8.4 Hz, 1H), 3.83 (t, J=8.8 Hz, 1H), 2.81-2.71 (m, 5H), 2.27-2.23 (m, 1H), 2.18-2.03 (m, 10H), 1.97-1.88 (m, 2H), 1.29-1.17 (m, 11H), 1.10 (s, 3H) ppm.

PTL-14-24:

Standard procedure was applied using 14-hydroxy-parthenolide (10 mg, 0.037 mmol), Rh₂(OAc)₄ (0.8 mg, 5 mol %) and ethyl 2-diazo-2-(2-(trifluoromethyl)-phenyl)-acetate (19 mg, 0.74 mmol). Isolated: 6 mg, 31% yield. ¹H NMR (400 MHz, CDCl₃): δ=7.68-7.48 (m, 8H), 6.32 (d, J=4.0 Hz, 2H), 5.60 (d, J=11.6 Hz, 2H), 5.46 (d, J=12.8 Hz, 2H), 5.23 (d, J=9.2 Hz, 2H), 4.29-4.03 (m, 9H), 3.87-3.74 (m, 3H), 2.78-2.76 (m, 4H), 2.45-1.88 (m, 12H), 1.30-1.17 (m, 14H) ppm.

PTL-14-25:

Standard procedure was applied using 14-hydroxy-parthenolide (10 mg, 0.037 mmol), Rh₂(OAc)₄ (0.8 mg, 5 mol %) and ethyl 2-diazo-2-(4-(trifluoromethyl)-phenyl)-acetate (19 mg, 0.74 mmol). Isolated: 8 mg, 42% yield. ¹H NMR (400 MHz, CDCl3): δ=7.64-7.61 (m, 4H), 7.54 (brs, 4H), 6.34 (s, 2H), 5.62 (s, 2H), 5.50 (brs, 2H), 4.90 (d, J=8.0 Hz, 2H), 4.26-4.06 (m, 8H), 3.89-3.80 (m, 2H), 2.80-2.72 (m, 6H), 2.26-2.05 (m, 10H), 1.90-1.85 (m, 2H), 1.34-1.13 (m, 14H) ppm.

PTL-14-26:

Standard procedure was applied using 14-hydroxy-parthenolide (10 mg, 0.037 mmol), Rh₂(OAc)₄ (0.8 mg, 5 mol %) and benzyl 2-diazo-2-phenylacetate (19 mg, 0.74 mmol), Isolated: 6 mg 32% yield. ¹H NMR (400 MHz, CDCl₃): δ=7.56-7.21 (m, 20H), 6.31 (s, 2H), 5.58 (brs, 2H), 5.41 (brs, 2H), 5.29 (brs, 4H), 4.90 (s, 1H), 4.87 (s, 1H), 4.16-4.04 (m, 4H), 3.77 (brs, 2H), 2.74 (brs, 6H), 2.27-2.05 (m, 10H), 1.86-1.84 (m, 2H), 1.19 (s, 3H), 1.08 (s, 3H) ppm.

PTL-14-27:

Standard procedure was applied using 14-hydroxy-parthenolide (10 mg, 0.037 mmol), Rh₂(OAc)₄ (0.8 mg, 5 mol %) and 2-morpholinoethyl 2-diazo-2-phenylacetate (20 mg, 0.74 mmol). Isolated: 9 mg, 46% yield. ¹H NMR (400 MHz, CDCl3): δ=7.43-7.30 (m, 10H), 6.32 (brs, 2H), 5.61-5.59 (m, 2H), 5.28 (brs, 2H), 5.17 (s, 1H), 4.85 (s, 1H), 4.36-4.08 (m, 9H), 4.05-3.89 (m, 2H), 3.56 (brs, 8H), 2.79-2.74 (m, 6H), 2.53 (brs, 8H), 2.41-2.07 (m, 16H), 1.29-1.24 (m, 6H), 1.10 (s, 1H) ppm.

### 6.7 Example 7

### Synthesis of 9,13-Disubstituted Parthenolide Derivatives

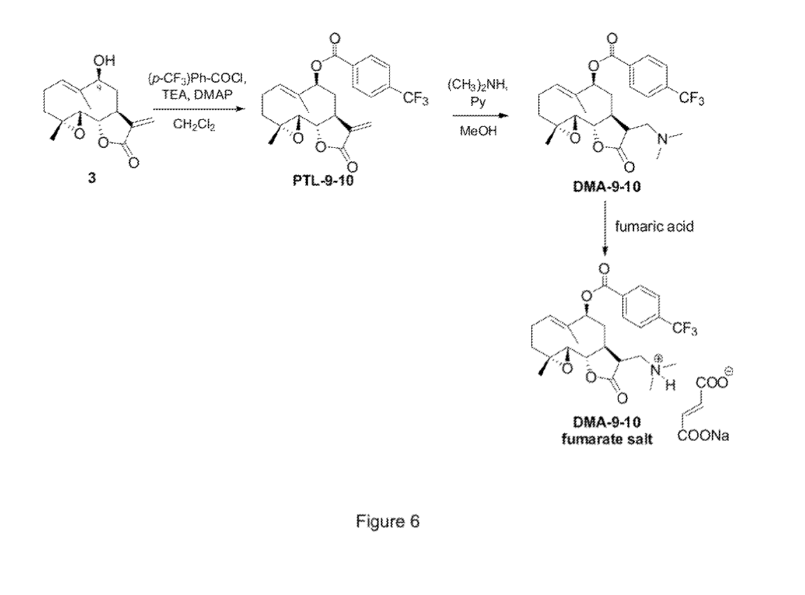

This example describes and demonstrates the preparation of compounds of general formula III according to the methods provided herein. In particular, it demonstrates how disubstituted parthenolide derivatives, such as 9,13-disubstituted parthenolide derivatives, can be prepared via chemoenzymatic functionalization of position C9 followed by chemical functionalization of position C13 (FIG. 6). Analogously, compounds of general formula IV can be prepared via chemoenzymatic functionalization of position C14 as described in Example 4 followed by similar procedures for C13 functionalization.

It is well known that the α-methylene-γ-lactone in parthenolide exhibits electrophilic reactivity and that the C13 site in this molecule can thus undergo Michael addition with nucleophilic reagents such as, for example, amine- or thiol-containing reagents. In particular, primary and secondary amines readily add to this site of the molecule (C13) under standard reaction conditions to yield C13-substituted amine-adducts. (Guzman, Rossi et al. 2006; Nasim and Crooks 2008; Neelakantan, Nasim et al. 2009) Although this type of modification was not found to lead to significant improvements in the anticancer activity of parthenolide, it can be useful to improve its limited water-solubility. (Guzman, Rossi et al. 2006; Nasim and Crooks 2008; Neelakantan, Nasim et al. 2009)

Since the chemoenzymatic functionalization at either the C9 or C14 of parthenolide as described herein are remote with respect to the C13 site. Both C9- and C14-substituted parthenolide analogs can be further modified at position C13, e.g., via nucleophilic addition of an amine reagent to the α-methylene-γ-lactone moiety, to yield C9,C13- and C13,C14-substituted parthenolide analogs within the scope of the invention. An exemplary procedure for the preparation of doubly substituted parthenolide derivatives of this type is provided in FIG. 6. Because of the presence of basic amino group in these molecules, salt forms of these molecules can be then prepared via addition of an appropriate acid (e.g., fumaric acid, FIG. 6), which could be beneficial to further improve the water solubility and oral bioavailability of these compounds.

To illustrate this aspect of the invention, an improved C9-modified parthenolide derivative, PTL-9-10, was made react with dimethylamine to yield the corresponding dimethylamino adduct, DMA-9-10, which was then converted to its fumarate salt (FIG. 6). This compound was found to retain comparable in vitro antileukemic activity as PTL-9-10 while being more than 100-fold more soluble in aqueous buffer.

Synthesis of DMA-9-010.

To a solution of 9-010 (30 mg, 0.07 mmol) in 10 mL of methanol under argon atmosphere was added dimethylamine 2 M in THF (103 μL, 0.21 mmol). The reaction was allowed to stir until completion (ca. 12 hrs.). After removal of the solvent under reduced pressure, purification of the residue by silica gel flash chromatography (12:1 dichloromethane:CMA, CMA=(40:9:1, Chloroform:methanol:ammonium hydroxide)) afforded 9-010-DMA (26 mg, 77% yield). ¹H NMR (500 MHz, CDCl₃): δ=1.26-1.32 (m, 1H), 1.35 (s, 3H), 1.84 (s, 3H), 2.10-2.24 (m, 3H), 2.25-2.44 (m, 8H), 2.45-2.58 (m, 3H), 2.72 (d, 1H, 9.0 Hz), 2.80-2.93 (m, 1H), 3.90 (t, 1H, J=7.8 Hz), 5.40 (d, 1H, J=8.8 Hz), 5.62 (d, 1H, J=11.7 Hz), 7.72 (d, 2H, J=7.8 Hz), 8.13 (d, 2H, J=7.8 Hz).

### 6.8 Example 8

### Antiproliferative Activity of the Parthenolide Derivatives

The parthenolide derivatives described in the examples above have been demonstrated to possess potent anticancer activity against various types of cancer cells, including leukemia and lymphomas. To evaluate their anticancer activity, these compounds were tested for cytotoxicity against acute myelogenous leukemia (AML), chronic lymphocytic leukemia (CLL), acute lymphoblastic leukemia (ALL), mantle cell lymphoma (MCL), diffuse large B-cell lymphoma cells, including primary AML, ALL, and CLL specimens. Primary AML specimens tested included two relapsed refractory AML specimens, which feature both a normal (AML100510) and a complex karyotype (AML123009), the latter exhibiting reduced sensitivity to PTL (LD₅₀: 9.7 vs. 6.1 μM, TABLE 2). Dose-response curves were obtained by measuring the variation of cell viability at increasing compound concentration using a previously described assay based on cell staining with annexin-V and 7-amino-actinomycin (7-ADD) followed by flow cytometry analysis (Guzman, Neering et al. 2001).

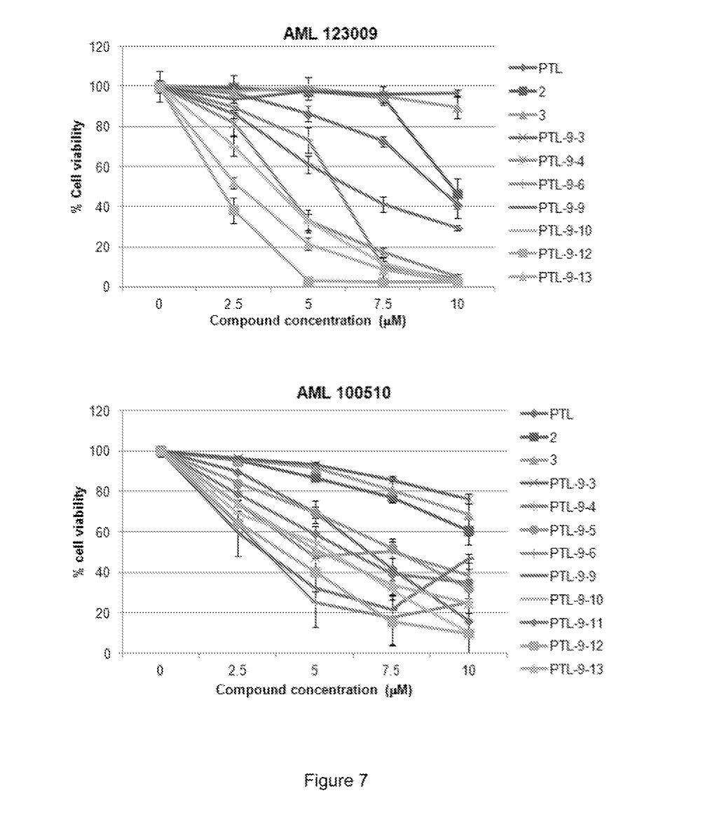

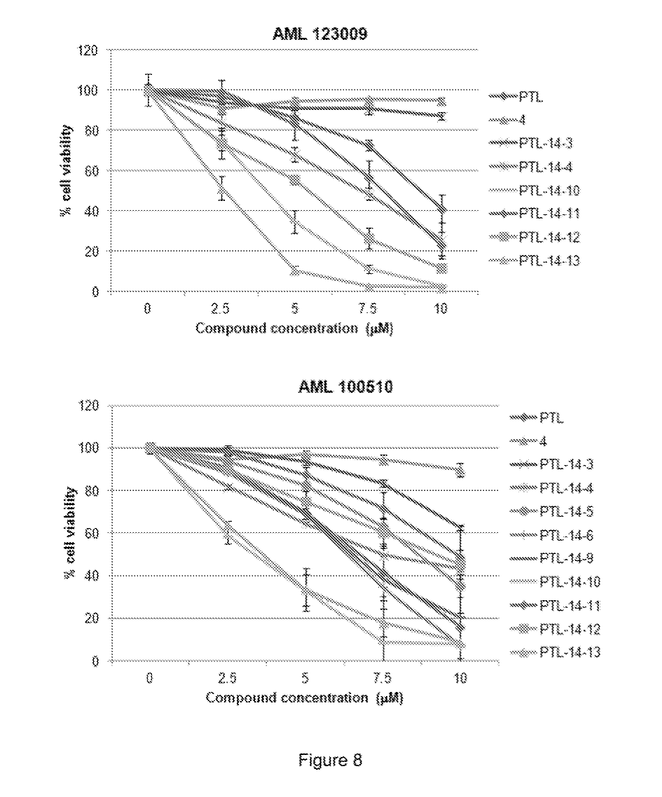

As illustrated in FIGS. 7 and 8 and TABLE 2, a dramatic and unexpected reduction in antileukemic potency was observed with the two hydroxylated derivatives 3 and 4 and with the acetylated derivatives PTL-9-3 and PTL-14-3. In stark contrast, the benzoylated derivatives PTL-9-4 and PTL-14-4, were found unexpectedly to exhibit a significantly improved activity (compared to PTL) against the complex-karyotype AML cells (AML123009), as indicated by the two-fold lower LD₅₀ values (TABLE 2, FIGS. 7-8). These results clearly showed the beneficial effect of larger, aromatic substituents at either the C9 or C14 sites toward potentiating PTL antileukemic activity. Accordingly, a set of compounds carrying variously substituted benzoyl groups at each of these positions (FIGS. 2 and 3). Notably, most of the resulting semisynthetic derivatives were found to be 2- to 3-fold more potent than parthenolide as illustrated by the dose-response curves in FIGS. 7 and 8, and as summarized in TABLE 2. Within the C9-functionalized series, the largest increases in potency were achieved through substitution of the aryl moiety at the para position with fluorine (PTL-9-9), a dimethylamino (PTL-9-6), or trifluoromethyl group (PTL-9-10). A similar structure-activity trend was observed for the C14-functionalized series of compounds, although in this case the para-trifluoromethyl-benzoyl substituted derivative, PTL-14-10, emerged as the most potent derivative in the context of both AML specimens.

The beneficial effect of increasing the lipophilicity of the aryl moiety further suggested the design of compounds PTL-9-12, PTL-9-13, PTL-14-12, and PTL-14-13. Notably, the addition of a second trifluoromethyl group to the benzoyl moiety brought about a further increase in antileukemic potency for both the C9- and C14 modified analogs and in particular against AML123009 cells. Overall, the most promising compounds within each series, namely PTL-9-12 (LD₅₀: 2.3 μM) and PTL-14-13 (LD₅₀: 2.5 μM), were found to exhibit a 4.2- and 3.9-fold enhanced cytotoxicity, respectively, against primary AML cells compared to PTL (LD₅₀: 9.7 μM, TABLE 2).

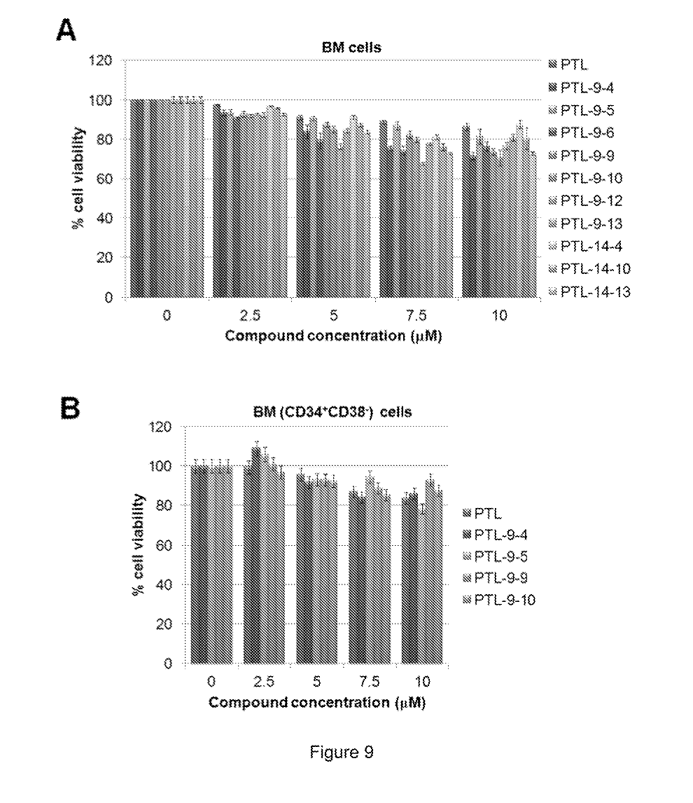

The most potent parthenolide derivatives identified in these studies were selected for further characterization to evaluate their selectivity against malignant over normal cells. For these studies, normal bone marrow cells (BM cells) obtained from healthy donors were utilized. Importantly, all these compounds, with the exception of PTL-9-6, did not significantly impart the viability of normal cells (FIG. 9A), thus presenting the desired high selectivity against leukemic cells. Remarkably, at a concentration sufficient to kill 98% of primary AML cells (10 μM), compounds PTL-9-12 and PTL-9-13 were found to cause only less than 15% reduction in the viability of normal BM cells. For some of the most promising PTL analogs, it was also possible to test their cytotoxic effect on the progenitor (CD34⁺CD38⁻) cell sub-population of the bone marrow samples (FIG. 9B). Notably, the compounds exhibited comparably low or even lower cytoxicity than in the context of mature BM cells. Taken together, these results showed that functionalizations at the C9/C14 sites are not only beneficial in enhancing PTL cytotoxicity, but also that such effect is exerted with high selectivity in the context of leukemic cells. As a result, the LD₅₀(BM)/LD₅₀(AML) ratio of the original compound could be improved by several folds by means of these chemoenzymatic manipulations (e.g., nearly 20 (PTL-9-12) and 40 (PTL-9-13) as compared to about 8 for PTL, TABLE 2).

TABLE 2
LD₅₀ values for parthenolide (PTL) and its chemoenzymatic
derivatives against the two primary AML specimens and
healthy bone marrow (BM) cells. The values in parenthesis
indicate relative activities compared to PTL.

| | | | LD₅₀ (μM) |
|---|---|---|---|
| | LD₅₀ (μM) | LD₅₀ (μM) | Bone |
| | AML123009 | AML100510 | Marrow |
| PTL | 9.7 (1) | 6.1 (1) | >80 |
| 2 | 13.5 (0.7) | 13.9 (0.4) | n.d. |
| 3 | 95 (0.1) | 17.4 (0.4) | n.d. |
| PTL-9-3 | >100 | 24.2 (0.3) | n.d. |
| PTL-9-4 | 4.1 (2.4) | 6.2 (1.0) | >20 |
| PTL-9-5 | 6.1 (1.6) | 7.2 (0.8) | >50 |
| PTL-9-6 | 4.8 (2.0) | 3.1 (2.0) | >50 |
| PTL-9-9 | 6.3 (1.5) | 2.7 (2.2) | 25 |
| PTL-9-10 | 3.5 (2.7) | 4.3 (1.4) | 23 |
| PTL-9-11 | 4.6 (2.1) | 6.3 (1.0) | n.d. |
| PTL-9-12 | 2.3 (4.2) | 3.7 (1.6) | 44 |
| PTL-9-13 | 2.7 (3.6) | 5.1 (1.2) | 105 |
| 4 | >100 | >100 | n.d. |
| PTL-14-3 | >100 | 12.5 (0.5) | n.d. |
| PTL-14-4 | 6.4 (1.5) | 7.8 (0.8) | >80 |
| PTL-14-5 | 20.8 (0.5) | 8.7 (0.7) | n.d. |
| PTL-14-6 | 5.9 (1.7) | 5.4 (1.1) | n.d. |
| PTL-14-9 | 6.4 (1.5) | 6.2 (1.0) | n.d. |
| PTL-14-10 | 3.7 (2.6) | 3.1 (2.0) | 37 |
| PTL-14-11 | 7.8 (1.2) | 10 (0.6) | n.d. |
| PTL-14-12 | 5 (1.9) | 9.5 (0.6) | n.d. |
| PTL-14-13 | 2.5 (3.9) | 3.4 (1.8) | 25 |

n.d. = not determined.

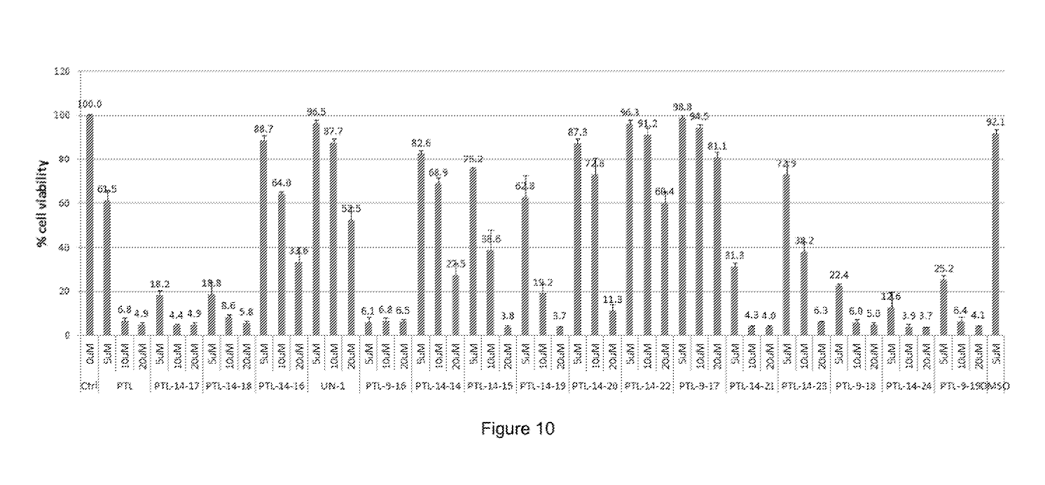

Based on the structure-activity information acquired as described above, additional parthenolide analogs were prepared according to the methods provided herein which contain variously substituted aromatic and heterocyclic substituents at position C9 (e.g., PTL-9-14 through PTL-9-16) or position C14 (e.g., PTL-14-14 through PTL-9-19). These compounds were tested for antileukemic activity in cell-based assay using M9-ENL1 leukemia cells (FIG. 10). Notably, a number of compounds, namely PTL-14-17, PTL-14-18, PTL-14-24, PTL-9-16, PTL-9-18, and PTL-9-19 showed 3- to 10-fold greater reduction of cell viability as compared to PTL at the 5 uM concentration (FIG. 10).

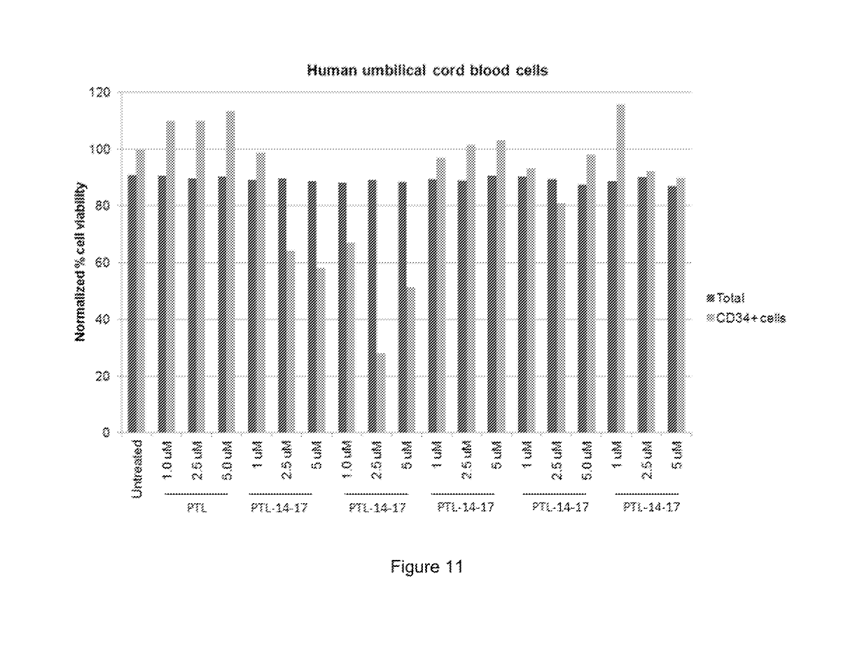

As described in EXAMPLE 6, other parthenolide derivatives were prepared via conversion of 9-hydroxy-parthenolide or 14-hydroxy-parthenolide with hydroxyl group functionalization strategies other than acylation. These include PTL-14-20 and PTL-14-21 prepared via hydroxyl group alkylation, and PTL-9-17 through PTL-9-21 and PTL-14-22 through PTL-14-27 prepared via metal-catalyzed O—H functionalization. Analysis of antileukemic activity of these compounds against M9-ENL1 leukemia cells revealed that many of these ether-substituted derivatives (e.g., PTL-9-18, PTL-14-24) were considerably more active than parthenolide (FIG. 10). Furthermore, the ether-substituted analog PTL-9-18 and PTL-9-19 showed no increase in cytotoxicity against either normal hematopoietic cells (=human umbilical cord blood cells), including the progenitor (CD34+) subpopulation of these cells (FIG. 11), thus exhibiting high selectivity against malignant cells. A particularly relevant result from the present studies is the discovery that the C9 and C14 sites represent two ‘hot spots’ for potentiating the antileukemic activity of parthenolide. Notably, the improvements in anticancer activity against AML cells could be achieved without increasing their cytotoxicity against normal hematopoietic cells, thereby effectively enhancing the therapeutic index of the molecule.

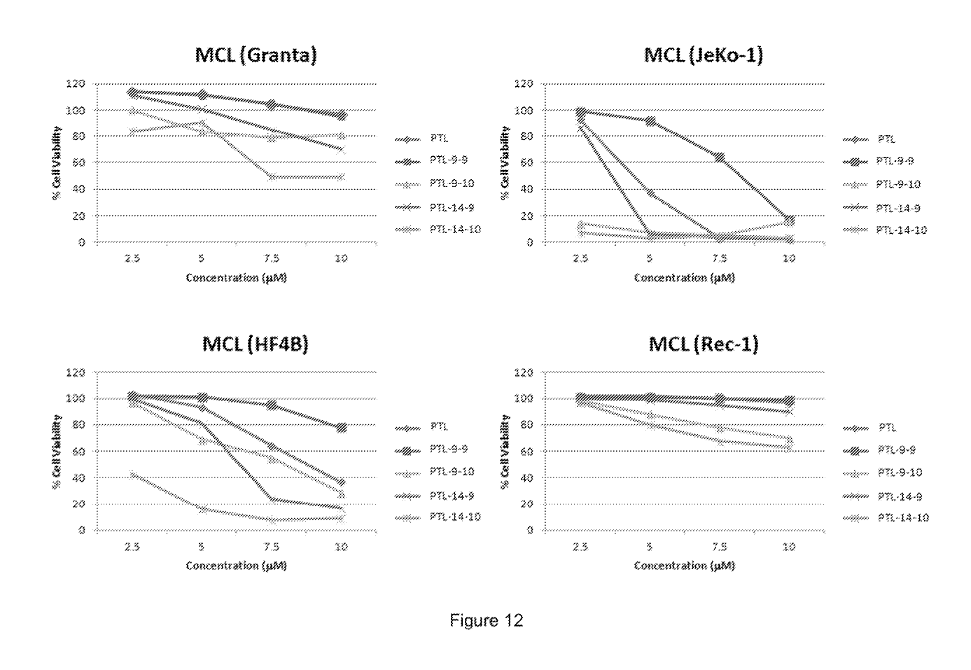

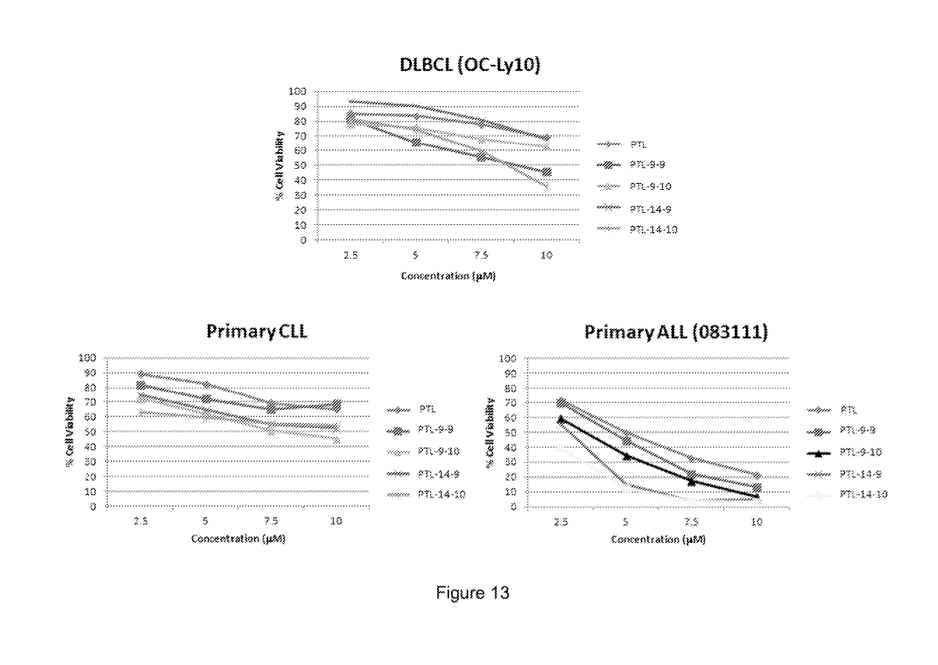

Selected parthenolide analogs were further tested for in vitro anticancer activity against other representative types of hematologic malignancies, such as mantle cell lymphoma (MCL), chronic lymphocytic leukemia (CLL), acute lymphoblastic leukemia (ALL), and large B-cell lymphoma. Also in this case, the C9- and C14-modified PTL derivatives were found to be able to induce more robust apoptosis than parthenolide (FIGS. 12 and 13), demonstrating their potential utility for the pharmacological treatment of these diseases.

Finally, the doubly functionalized PTL derivatives, DMA-9-010 (FIG. 6), was also tested for cytotoxic activity against primary AML cells. Interestingly, this compound was found to have comparable in-vitro activity to PTL derivative PTL-9-10 while being 100-fold more water-soluble. These results further demonstrate the possibility of obtaining PTL derivatives which combine improved anticancer activity with increased water-solubility using the methods provided herein. This aspect of the invention can be also useful toward the discovery of parthenolide-based anticancer agents with improved pharmacological and pharmacokinetic (e.g., oral bioavailability) properties.

Experimental Procedures.

Biological activity studies were performed using cell-based assays with human leukemia cells (M9-ENL1), mantle cell lymphoma (MCL) cells (Granta, JeKo-1, HF4B, and Rec-1), diffuse large B-cell lymphoma (DLBCL) cells (OC-Ly10), and primary acute myeloid leukemia (AML), primary acute lymphoblastic leukemia (ALL), and chronic lymphocytic leukemia (CLL) cells. Primary AML, ALL, and CLL cells and normal bone marrow (BM) cells were all obtained with informed consent from volunteer donors. In some cases, cells were cryopreserved in freezing medium of Iscove modified Dulbecco medium (IMDM), 40% fetal bovine serum (FBS), and 10% dimethylsulfoxide (DMSO) or in CryoStor CS-10 (VWR, West Chester, Pa.). Cells were cultured in serum-free medium (SFM)19 for 1 hour before the addition of parthenolide or its derivatives. Apoptosis assays were performed as described in (Guzman, Neering et al. 2001). Briefly, after 24 hours of treatment, cells were stained for the surface antibodies CD38-allophycocyanin (APC), CD34-PECy7, and CD123-phycoerythin (Becton Dickinson, San Jose, Calif.) for 15 minutes. Cells were washed in cold PBS and resuspended in 200 μL of annexin-V buffer (0.01 M HEPES/NaOH, 0.14 M NaCl, and 2.5 mM CaCl₂). Annexin-V-fluorescein isothiocyanate (FITC) and 7-aminoactinomycin (7-AAD; Molecular Probes, Eugene, Oreg.) were added, and the tubes were incubated at room temperature for 15 minutes then analyzed on a BD LSRII flow cytometer (BD Biosciences, San Jose, Calif.). Analyses for phenotypically described stem cell subpopulations were performed by gating CD34⁺/CD38⁻ populations. Viable cells were scored as Annexin-V negative/7-AAD negative. The percent viability data provided are normalized to untreated control specimens.

### REFERENCES

Each reference listed below is hereby incorporated by reference herein in its entirety.
Anderson, K. N. and B. E. Bejcek (2008). J Pharmacol Sci 106(2): 318-320.
Baeuerle, P. A. and T. Henkel (1994). Annual Review of Immunology 12: 141-179.
Barnes, P. J. and I. M. Adcock (1997). Trends in Pharmacological Sciences 18(2): 46-50.
Barnes, P. J. and M. Larin (1997). New England Journal of Medicine 336(15): 1066-1071.
Bessho, Y., D. R. Hodgson, et al. (2002). Nat Biotechnol 20(7): 723-728.
Bonnet, D. and J. E. Dick (1997). Nat Med 3(7): 730-737.
Castaneda-Acosta, J., N. H. Fischer, et al. (1993). J Nat Prod 56(1): 90-98.
Cheng, G. and L. Xie (2011). Molecules 16(8): 6758-6768.
Cirino, P. C. and F. H. Arnold (2003). Angew. Chem. Int. Ed. Engl. 42(28): 3299-3301.
Costello, R. T., F. Mallet, et al. (2000). Cancer Research 60(16): 4403-4411.
Cox, C. V., R. S. Evely, et al. (2004). Blood 104(9): 2919-2925.
Cox, G. G., J. J. Kulagowski, et al. (1992). Synlett (12): 975-976.
Dedkova, L. M., N. E. Fahmi, et al. (2003). Journal of the American Chemical Society 125(22): 6616-6617.
Estabrook, N. C., H. Chin-Sinex, et al. (2011). Free Radic Biol Med 51(12): 2249-2258.
Galal, A. M., A. R. S. Ibrahim, et al. (1999). Phytochemistry 51(6): 761-765.
Garcia-Pineres, A. J., V. Castro, et al. (2001). Journal of Biological Chemistry 276(43): 39713-39720.
Garcia-Pineres, A. J., M. T. Lindenmeyer, et al. (2004). Life Sci 75(7): 841-856.
Ghantous, A., H. Gali-Muhtasib, et al. (2010). Drug Discov Today 15(15-16): 668-678.
Gopal, Y. N., T. S. Arora, et al. (2007). Chem Biol 14(7): 813-823.
Gopal, Y. N., E. Chanchorn, et al. (2009). Mol Cancer Ther 8(3): 552-562.
Graham, S. M., H. G. Jorgensen, et al. (2002). Blood 99(1): 319-325.
Guan, Y. H., B. Gerhard, et al. (2003). Blood 101(8): 3142-3149.
Guzman, M. L., L. Karnischky, et al. (2004). Blood 104(11): 697a-697a.
Guzman, M. L., S. J. Neering, et al. (2001). Blood 98(8): 2301-2307.
Guzman, M. L., R. M. Rossi, et al. (2005). Blood 105(11): 4163-4169.
Guzman, M. L., R. M. Rossi, et al. (2006). Blood 108(11): 74a-74a.
Guzman, M. L., C. F. Swiderski, et al. (2002). Proc Natl Acad Sci USA 99(25): 16220-16225.
Han, C., F. J. Barrios, et al. (2009). J. Org. Chem. 74(18): 7176-7179.
Hehner, S. P., M. Heinrich, et al. (1998). J Biol Chem 273(3): 1288-1297.
Heptinstall, S., A. White, et al. (1985). Lancet 1(8437): 1071-1074.
Hirakawa, H. and T. Nagamune (2010). Chembiochem 11(11): 1517-1520.
Holcomb, B. K., M. T. Yip-Schneider, et al. (2012). J Gastrointest Surg 16(7): 1333-1340.
Holyoake, T., X. Y. Jiang, et al. (1999). Blood 94(6): 2056-2064.
Hwang, D., N. H. Fischer, et al. (1996). Biochem Biophys Res Commun 226(3): 810-818.
Hwang, D. R., C. W. Chang, et al. (2006). Bioorganic & Medicinal Chemistry 14(1): 83-91.
Idris, A. I., H. Libouban, et al. (2009). Mol Cancer Ther 8(8): 2339-2347.
Janecka, A., A. Wyrebska, et al. (2012). Drug Discov Today 17(11-12): 561-572.
Juliana, C., T. Fernandes-Alnemri, et al. (2010). J Biol Chem 285(13): 9792-9802.
Kawasaki, B. T., E. M. Hurt, et al. (2009). Prostate 69(8): 827-837.
Killmann, S. A. (1991). Baillieres Clin Haematol 4(3): 577-598.
Kim, I. H., S. W. Kim, et al. (2012). Exp Mol Med 44(7): 448-456.
Kim, J. H., L. Liu, et al. (2005). Cancer Res 65(14): 6312-6320.
Knight, D. W. (1995). Nat Prod Rep 12(3): 271-276.
Kourouklis, D., H. Murakami, et al. (2005). Methods 36(3): 239-244.
Kreuger, M. R., S. Grootjans, et al. (2012). Anticancer Drugs 23(9): 883-896.
Kwok, B. H., B. Koh, et al. (2001). Chem Biol 8(8): 759-766.
Landwehr, M., M. Carbone, et al. (2007). Chem. Biol. 14(3): 269-278.
Lapidot, T., C. Sirard, et al. (1994). Nature 367(6464): 645-648.
Lesiak, K., K. Koprowska, et al. (2010). Melanoma Res 20(1): 21-34.
Li, S. Y., L. M. Podust, et al. (2007). J. Am. Chem. Soc. 129(43): 12940-12941.
Li, Y., Y. Zhang, et al. (2012). Mol Med Rep 6(3): 477-482.
Liu, C. C. and P. G. Schultz (2010). Annu. Rev. Biochem. 79: 413-444.
Liu, Y., W. L. Lu, et al. (2008). J Control Release 129(1): 18-25.
Liu, Z., S. Liu, et al. (2009). J Pharmacol Exp Ther 329(2): 505-514.
Merfort, I. (2011). Current Drug Targets 12(11): 1560-1573.
Murakami, H., A. Ohta, et al. (2006). Nat Methods 3(5): 357-359.
Nakshatri, H., S. E. Rice, et al. (2004). Oncogene 23(44): 7330-7344.
Nasim, S. and P. A. Crooks (2008). Bioorg Med Chem Lett 18(14): 3870-3873.
Neelakantan, S., S. Nasim, et al. (2009). Bioorg. Med. Chem. Lett. 19(15): 4346-4349.
Neukirch, H., N. C. Kaneider, et al. (2003). Bioorg Med Chem 11(7): 1503-1510.
Neumann, H., K. Wang, et al. (2010). Nature 464(7287): 441-444.
Oka, D., K. Nishimura, et al. (2007). International Journal of Cancer 120(12): 2576-2581.
Otey, C. R., M. Landwehr, et al. (2006). PLoS Biol. 4(5): e112.
Park, J. H., L. Liu, et al. (2005). Cancer Res 65(7): 2804-2814.
Patel, N. M., S. Nozaki, et al. (2000). Oncogene 19(36): 4159-4169.
Peddibhotla, S., Y. J. Dang, et al. (2007). Journal of the American Chemical Society 129(40): 12222-12231.
Ralstin, M. C., E. A. Gage, et al. (2006). Mol Cancer Res 4(6): 387-399.
Rodriguez, E. A., H. A. Lester, et al. (2006). Proc Natl Acad Sci USA 103(23): 8650-8655.
Shanmugam, R., P. Kusumanchi, et al. (2011). Int J Cancer 128(10): 2481-2494.
Shanmugam, R., P. Kusumanchi, et al. (2010). Prostate 70(10): 1074-1086.
Siedle, B., A. J. Garcia-Pineres, et al. (2004). J Med Chem 47(24): 6042-6054.
Skalska, J., P. S. Brookes, et al. (2009). PLoS One 4(12): e8115.
Steele, A. J., D. T. Jones, et al. (2006). Leukemia 20(6): 1073-1079.
Sun, C. M., W. J. Syu, et al. (2003). J Nat Prod 66(9): 1175-1180.
Sun, Y., D. K. St Clair, et al. (2007). Mol Cancer Ther 6(9): 2477-2486.
Sun, Y., D. K. St Clair, et al. (2010). Cancer Res 70(7): 2880-2890.
Suvannasankha, A., C. D. Crean, et al. (2008). Clin Cancer Res 14(6): 1814-1822.
Sweeney, C. J., S. Mehrotra, et al. (2005). Mol Cancer Ther 4(6): 1004-1012.
Tiuman, T. S., T. Ueda-Nakamura, et al. (2005). Antimicrobial Agents and Chemotherapy 49(1): 176-182.
Tran, N. H., N. Huynh, et al. (2011). Chem Commun (Camb) 47(43): 11936-11938.
van der Donk, W. A. and H. Zhao (2003). Curr Opin Biotechnol 14(4): 421-426.
van Rhenen, A., N. Feller, et al. (2005). Clin Cancer Res 11(18): 6520-6527.
Wang, W., M. Adachi, et al. (2006). Apoptosis 11(12): 2225-2235.
Wang, W., M. Adachi, et al. (2009). Pancreas 38(4): e114-123.
Wen, J., K. R. You, et al. (2002). J Biol Chem 277(41): 38954-38964.
Won, Y. K., C. N. Ong, et al. (2005). Carcinogenesis 26(12): 2149-2156.
Won, Y. K., C. N. Ong, et al. (2004). Carcinogenesis 25(8): 1449-1458.
Woods, J. R., H. Mo, et al. (2011). J Med Chem 54(22): 7934-7941.
Wyrebska, A., K. Gach, et al. (2012). Chem Biol Drug Des 79(1): 112-120.
Yip-Schneider, M. T., H. Nakshatri, et al. (2005). Mol Cancer Ther 4(4): 587-594.
Yip-Schneider, M. T., H. Wu, et al. (2008). Pancreas 37(3): e45-53.
Zanotto-Filho, A., E. Braganhol, et al. (2011). Biochemical Pharmacology 81(3): 412-424.
Zhang, D., L. Qiu, et al. (2009). Mol Cancer Res 7(7): 1139-1149.
Zhang, K., S. El Damaty, et al. (2011). J. Am. Chem. Soc. 133(10): 3242-3245.
Zhang, K., B. M. Shafer, et al. (2012). J Am Chem Soc 134(45): 18695-18704.
Zhang, S., C. N. Ong, et al. (2004). Cancer Lett 208(2): 143-153.
Zhang, S., C. N. Ong, et al. (2004). Cancer Lett 211(2): 175-188.
Zhao, H. and W. A. van der Donk (2003). Curr Opin Biotechnol 14(6): 583-589.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A compound of formula (I) or formula (II) 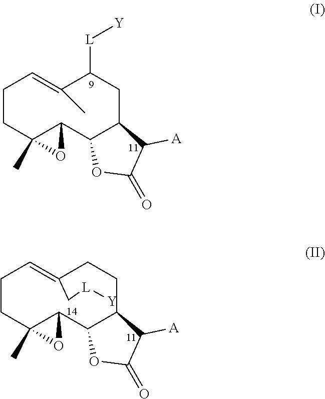
wherein
A is ═CH₂ or —CH₂R* wherein R* is an amino acid residue bonded to the A methylene via a nitrogen or sulfur atom; or R* is —NR¹R², —NR¹C(O)R², —NR¹CO₂R², or —SR¹, wherein R¹ and R² are independently selected from the group consisting of H and an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or where R* is —NR¹R², R₁ and R₂ optionally together with the nitrogen atom form a an optionally substituted 5-12 membered ring, the ring optionally comprising at least one heteroatom or group selected from —CO—, —SO—, —SO₂—, and —PO—;
L is —O—, —NH—, —NHC(O)—, —OC(O)—, —OC(O)NH—, —S—, —SO—, —SO₂—, —PO—, —OCH₂—, or a chemical bond connecting the carbon atom to Y; and Y represents a hydrogen atom, an optionally substituted alkyl, alkenyl, or alkynyl group, an optionally substituted heteroalkyl, heteroalkenyl, or heteroalkynyl group, an optionally substituted aryl group, an optionally substituted heteroaryl group, or an optionally substituted heterocyclic group; or
Y is absent and L represents a halogen atom, an azido group (—N₃), an optionally substituted triazole group, or a group —NR³R⁴, where R³ represents a hydrogen atom or an optionally substituted alkyl, alkenyl, or alkynyl group; R⁴ represents an optionally substituted alkyl, alkenyl, alkynyl, aryl, or heteroaryl group; or where R₃ and R₄ are connected together to form an optionally substituted heterocyclic group;
or a pharmaceutically acceptable salt thereof.

2. The compound of claim 1, wherein L is —OC(O)—, Y is selected from the group consisting of phenyl, 4-pyridyl, (4-dimethylamino)phenyl, para-, meta-, and ortho-fluoro-phenyl, para-, meta-, and ortho-trifluoromethyl-phenyl, (2,4-bis-trifluoromethyl)phenyl, (3,5-bis-trifluoromethyl)phenyl, 1- and 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene, and A is ═CH₂.

3. The compound of claim 1, wherein L is —OC(O)—, Y is selected from the group consisting of phenyl, 4-pyridyl, (4-dimethylamino)phenyl, para-, meta-, and ortho-fluoro-phenyl, para-, meta-, and ortho-trifluoromethyl-phenyl, (2,4-bis-trifluoromethyl)phenyl, (3,5-bis-trifluoromethyl)phenyl, 1- and 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene, and A is —CH₂R*, where R* is selected from the group consisting of methylamino (—NH(CH₃)), dimethylamino (—N(CH₃)₂), methylethylamino (—N(CH₃)(CH₂CH₃)), methylpropylamino (—N(CH₃)(CH₂CH₂CH₃)), methylisopropylamino (—N(CH₃)(CH₂(CH₃)₂), —N(CH₃)(CH₂CH₂OH), pyrrolidine, piperidine, 4-methylpiperidine, 1-phenylmethanamine (—NCH₂Ph), and 2-phenylethanamine (—NCH₂CH₂Ph).

4. The compound of claim 1, wherein L is —O—, Y is selected from the group consisting of (phenyl)methyl, (4-pyridyl)methyl, (4-dimethylaminophenyl)methyl, (para-, meta-, and ortho-fluoro-phenyl)methyl, (para-, meta-, and ortho-trifluoromethyl-phenyl)methyl, (2,4-bis-trifluoromethyl-phenyl)methyl, (3,5-bis-trifluoromethyl-phenyl)methyl, (naphyl)methyl, (3-N-methyl-indolyl)methyl, (5-(4-chlorophenyl)isoxazolyl)methyl, (2-(4-bromophenyl)furanyl)methyl, (2-(2-(trifluoromethyl)phenyl)furanyl)methyl, methyl(thiophene) and —CH(Ar′)COOR′ group, where Ar′ is selected from the group consisting of phenyl, 4-pyridyl, (4-dimethylamino)phenyl, para-, meta-, and ortho-fluoro-phenyl, para-, meta-, and ortho-trifluoromethyl-phenyl, (2,4-bis-trifluoromethyl)phenyl, (3,5-bis-trifluoromethyl)phenyl, 1- and 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene group, and R′ is selected from the group consisting of methyl, ethyl, propyl, isopropyl, tert-butyl, benzyl, 2-morpholinoethyl, 2-morpholinoethyl, 2-(piperidin-1-yl)ethyl, and 2-(pyrrolidin-1-yl)ethyl; and A is ═CH₂.

5. The compound of claim 1, wherein L is —O—, Y is selected from the group consisting of (phenyl)methyl, (4-pyridyl)methyl, (4-dimethylaminophenyl)methyl, (para-, meta-, and ortho-fluoro-phenyl)methyl, (para-, meta-, and ortho-trifluoromethyl-phenyl)methyl, (2,4-bis-trifluoromethyl-phenyl)methyl, (3,5-bis-trifluoromethyl-phenyl)methyl, (naphyl)methyl, (3-N-methyl-indolyl)methyl, (5-(4-chlorophenyl)isoxazolyl)methyl, (2-(4-bromophenyl)furanyl)methyl, (2-(2-(trifluoromethyl)phenyl)furanyl)methyl, methyl(thiophene) and —CH(Ar′)COOR′ group, where Ar′ is selected from the group consisting of phenyl, 4-pyridyl, (4-dimethylamino)phenyl, para-, meta-, and ortho-fluoro-phenyl, para-, meta-, and ortho-trifluoromethyl-phenyl, (2,4-bis-trifluoromethyl)phenyl, (3,5-bis-trifluoromethyl)phenyl, 1- and 2-naphyl, 3-N-methyl-indolyl, 5-(4-chlorophenyl)isoxazolyl, 2-(4-bromophenyl)furanyl, 2-(2-(trifluoromethyl)phenyl)furanyl, and thiophene group, and R′ is selected from the group consisting of methyl, ethyl, propyl, isopropyl, tert-butyl, benzyl, 2-morpholinoethyl, 2-morpholinoethyl, 2-(piperidin-1-yl)ethyl, and 2-(pyrrolidin-1-yl)ethyl; and A is —CH₂R*, where R* is selected from the group consisting of methylamino (—NH(CH₃)), dimethylamino (—N(CH₃)₂), methylethylamino (—N(CH₃)(CH₂CH₃)), methylpropylamino (—N(CH₃)(CH₂CH₂CH₃)), methylisopropylamino (—N(CH₃)(CH₂(CH₃)₂), N(CH₃)(CH₂CH₂OH), pyrrolidine, piperidine, 4-methylpiperidine, 1-phenylmethanamine (—NCH₂Ph), and 2-phenylethanamine (—NCH₂CH₂Ph).

6. A method for inhibiting cancer cell growth, the method comprising the step of administering to a mammal afflicted with cancer an amount of a compound of claim 1 effective to inhibit the growth of the cancer cells.

7-11. (canceled)

12. An engineered cytochrome P450 polypeptide having an improved enzyme capability, as compared to a P450 enzyme of SEQ ID NO: 1, SEQ ID NO: 2, or SEQ ID NO: 3, to hydroxylate parthenolide, wherein the engineered cytochrome P450 polypeptide comprises an amino acid sequence that is at least 60% identical to SEQ ID NO: 1, 2 or 3.

13. The polypeptide of claim 12 wherein the improved enzyme capability of the polypeptide is an improvement in its catalytic activity, coupling efficiency, regioselectivity and/or stereo selectivity.

14. The polypeptide of claim 12, wherein the polypeptide comprises an amino acid sequence that is at least 90% identical to SEQ ID NO: 1 and comprises an amino acid substitution at a position selected from the group consisting of position X26, X27, X43, X48, X52, X53, X73, X75, X76, X79, X82, X83, X88, X89, X95, X97, X143, X146, X176, X181, X182, X185, X189, X198, X206, X226, X227, X237, X253, X256, X261, X264, X265, X268, X269, X291, X320, X331, X329, X330, X354, X355, X367, X394, X435, X436, X444, X446, X438, and X439 of SEQ ID NO: 1.

15. The polypeptide of claim 12, wherein the polypeptide comprises an amino acid sequence that is at least 90% identical to SEQ ID NO: 2 and comprises an amino acid substitution at a position selected from the group consisting of position X28, X29, X45, X50, X54, X55, X75, X77, X78, X81, X83, X85, X90, X91, X97, X99, X145, X148, X178, X183, X184, X187, X191, X200, X208, X228, X229, X240, X256, X259, X264, X267, X268, X271, X272, X294, X323, X334, X332, X333, X358, X359, X371, X398, X439, X440, X448, X440, X442, and X443 of SEQ ID NO: 2.

16. The polypeptide of claim 12, wherein the polypeptide comprises an amino acid sequence that is at least 90% identical to SEQ ID NO: 3 and comprises an amino acid substitution at a position selected from the group consisting of position X29, X30, X46, X51, X55, X56, X76, X78, X79, X82, X85, X86, X91, X92, X99, X101, X147, X151, X180, X185, X186, X189, X193, X202, X210, X230, X231, X241, X257, X260, X265, X268, X269, X272, X273, X295, X324, X335, X333, X334, X365, X366, X378, X405, X446, X447, X455, X457, X449, and X450 of SEQ ID NO: 3.

17. The polypeptide of claim 12 wherein the improved capability is an improved capability to hydroxylate position 9, position 14, or both of these positions in parthenolide.

18. The polypeptide of claim 12, wherein the polypeptide is selected from the group consisting of SEQ ID NOS: 4, 5, 6, 7, 8, 9, 10, 11, 12, 13, 14, 15, 16, 17, 18, 19, and 20.

19. The polypeptide of claim 14, wherein the polypeptide comprises at least one of the features selected from the group consisting of: X48 is R or C; X53 is L or I; X75 is A, P, V, or T; X79 is V, A, T, N, or F; X82 is F, I, A, S, or W; X83 is A, L, S, V, or T; X88 is F, A, I, S, or V; X95 is K or I; X139 is H or Y; X143 is P or S; X176 is T or I; X181 is A or T; X182 is L or A; X185 is A, V or S; X189 is L or P; X198 is A or V; X206 is F or C; X227 is S or R; X237 is Q or H; X253 is G or E; X256 is S or R; X291 is V or A; X329 is V or A; X354 is V or L; X367 is I or V; X464 is E or G; and X710 is I or T.

20. The polypeptide of claim 15, wherein the polypeptide comprises at least one of the features selected from the group consisting of: X81 is V or A; X85 is A or P; X90 is F or A; X184 is L or A; and X187 is A or L.

21. The polypeptide of claim 12, wherein the polypeptide comprises an amino acid sequence comprising a cytochrome P450 heme domain that is at least 60% identical to the amino acid sequence from X1 to X500 in SEQ ID NO: 1, 2, 3, 4, 5, 6, 7, 8, 9, 10, 11, 12, 13, 14, 15, 16, 17, 18, 19, and/or 20.

22. A method for hydroxylating parthenolide, comprising the steps of:
(a) contacting parthenolide with a cytochrome P450 polypeptide of claim 1 under reaction conditions suitable for catalyzing hydroxylation of parthenolide;
(b) catalyzing the hydroxylation of parthenolide, while preserving the -methylene-lactone moiety therein, thereby producing an hydroxylated derivative of parthenolide; and
(c) isolating the hydroxylated derivative of parthenolide.

23. The method of claim 22, wherein the hydroxylated products comprise at least one compounds selected from the group consisting of 14-hydroxyparthenolide, 9(S)-hydroxyparthenolide, and 9(R)-hydroxyparthenolide.

24. The method of claim 22, wherein the polypeptide is tethered to a solid support.

25. The method of claim 22, wherein the polypeptide is contained in a host cell.
